(19) United States
(12) Patent Application Publication
(10) Pub. No.: US 2025/0127802 A1
(43) Pub. Date: Apr. 24, 2025

(54) HALIDE-FREE AMMONIUM SILANES

(71) Applicant: TOPIKOS SCIENTIFIC, INC., Fort Wayne, IN (US)

(72) Inventors: Gary Allred, Wake Forest, NC (US); Lanny Liebeskind, Atlanta, GA (US); William R. Cast, Fort Wayne, IN (US); Carl Hilliard, Henderson, NV (US)

(73) Assignee: TOPIKOS SCIENTIFIC, INC., Fort Wayne, IN (US)

(21) Appl. No.: 18/936,782

(22) Filed: Nov. 4, 2024

Related U.S. Application Data
(63) Continuation of application No. PCT/US2023/021069, filed on May 4, 2023.
(60) Provisional application No. 63/338,400, filed on May 4, 2022.

(51) Int. Cl. A61K 31/695; A61K 47/18; A61K 47/20

(52) U.S. Cl. CPC A61K 31/695; A61K 47/18; A61K 47/20

(57) ABSTRACT

Provided herein are ammonium silanes, compositions, their formulations, including powdered and solid formulations, and methods of use to treat infections in humans and animals. The ammonium silanes and compositions described herein can also be used as a preservative, disinfectant, or in another anti-microbial use.

### CROSS-REFERENCE TO RELATED APPLICATIONS

This application is a continuation of International Application No. PCT/US2023/021069, filed with the U.S. Receiving Office on May 4, 2023, which claims the benefit of U.S. Provisional Application 63/338,400, filed May 4, 2022. The entirety of each of these applications is hereby incorporated by reference for all purposes.

### FIELD OF THE INVENTION

This disclosure provides compounds and compositions useful for reducing, preventing, or inhibiting the growth and development of microbial organisms, including as biofilms. The compounds and compositions described herein are useful: to treat or prevent harmful microbial infections, including polymicrobial infections, on or in a host such as an animal or human; in cleaning, sanitizing, and disinfecting various types of surfaces; as preservatives or additives to prevent contamination, infection, decomposition, or spoilage due to microbes; and as pesticides to treat destructive biofilm infections.

### BACKGROUND OF THE INVENTION

Microorganisms include bacteria, fungi, viruses, and other single cell organisms. These microbes are frequently found as cells in communities of single and mixed or polymicrobial species called biofilms. Biofilms are complex surface attached communities of microorganisms held together by self-produced polymer matrices mainly composed of polysaccharides, secreted proteins, and extracellular DNAs (Tremblay et al., (2013). Method to grow Actinobacillus pleuropneumoniae biofilm on a biotic surface. BMC Vet. Res. 9:213). A biofilm can consist of a single microbial species or a combination of different species of bacteria, protozoa, archaea, algae, filamentous fungi, and yeast that strongly attach to each other and to biotic or abiotic surfaces (Raghupathi et al., (2017)), or at air-liquid interface. Synergistic interactions within a multispecies biofilm enhance individual species protection against grazing by a pelagic protozoan (Front. Microbiol. 8:2649. The ability of microorganisms to develop biofilms has been shown to be an adaptable attribute of microbes (Koczan et al., (2011). Cell surface attachment structures contribute to biofilm formation and xylem colonization by Erwinia amylovora. Appl. Environ. Microbiol. 77, 7031-7039). The formation of biofilm appears to be an evolutionarily-adapted phenomenon that provides microorganisms with strengthened survival mechanisms when compared with individual planktonic cells (Dang and Lovell (2016). Microbial surface colonization and biofilm development in marine environments. Microbiol. Mol. Biol. Rev. 80, 91-138), including enhanced ability to grow in oligotrophic environments (Bowden and Li (1997). Nutritional influences on biofilm development. Adv. Dent. Res. 11, 81-99.), greater access to nutritional resources (Dang and Lovell, 2016), improved survival to biocides (Flemming et al. (2016). Biofilms: an emergent form of bacterial life. Nat. Rev. Microbiol. 14, 563-575), enhanced organism productivity and interactions (Roder et al. (2018). Enhanced bacterial mutualism through an evolved biofilm phenotype. ISME J. 12, 2608-2618), as well as greater environmental stability (Dang and Lovell, 2016). It is readily apparent that biofilms provide novel mechanisms of protection for communities of bacterial species under adverse environmental conditions.

Biofilm formation is a complex process and can be described in five main phases: (i) reversible attachment phase, where microbes such as bacteria non-specifically attach to surfaces; (ii) irreversible attachment phase, which involves interaction between microbial cells and a surface using, for example, bacterial adhesins such as fimbriae and lipopolysaccharide (LPS); (iii) production of extracellular polymeric substances (EPS) by the resident microbial cells; (iv) biofilm maturation phase, in which microbe cells synthesize and release signaling molecules to sense the presence of each other, conducing to the formation of microcolony and maturation of biofilms; and (v) dispersal/detachment phase, where the microbial cells depart biofilms and comeback to independent planktonic lifestyle (Muhammad et al., Beyond Risk: Bacetrial Biofilms and Their Regulating Approaches. Front. Microbiol. 21 May 2020).

In the healthcare settings, biofilms have been shown to develop on medical device surfaces, dead tissues (e.g., sequestra of bones), and inside living tissues (e.g., lung tissue, teeth surfaces (Alav et al. (2018). Role of bacterial efflux pumps in biofilm formation. J. Antimicrob. Chemother. 73, 2003-2020). Biofilms can lead to biofouling, contamination, and/or corrosion of devices. For example, biofilms may develop on the surface of biomedical devices such as catheters, prosthetic heart valves, pacemakers, breast implants, contact lenses, and cerebrospinal fluid shunts (Wu et al., (2015). Strategies for combating bacterial biofilm infections. Int. J. Oral Sci. 7, 1-7.). The development of biofilms on biomedical devices poses significant health risks. For example, there are nearly 80,000 central venous catheter associated bloodstream infections that occur each year in the U.S. with a 12-25% mortality rate, and eighty percent of urinary tract infections are directly linked to catheterization.

Both Gram-positive and Gram-negative bacteria may attach to and develop biofilms on the surfaces of these devices, but the most frequently reported biofilm forming bacteria are Staphylococcus aureus, Staphylococcus epidermidis and Pseudomonas aeruginosa (Pakharukova et al. (2018). Structural basis for Acinetobacter baumannii biofilm formation. Proc. Natl. Acad. Sci. U.S.A. 115, 5558-5563). It is estimated that about two-thirds of indwelling devices related infections are caused by the staphylococcal species (Khatoon et. al.., (2018). Bacterial biofilm formation on implantable devices and approaches to its treatment and prevention. Heliyon 4:e01067.; Masters et al., (2019) Evolving concepts in bone infection: redefining “biofilm”, “acute vs. chronic osteomyelitis”, “the immune proteome” and “local antibiotic therapy”. Bone Res. 7:20).

In addition, microbial biofilms act as a reservoir that seed disseminated infections, and contribute to chronic infections and diseases in humans and animals such as cystic fibrosis (CF), otitis media, periodontitis, infective endocarditis (IE), chronic wounds, persistent mating-induced endometritis (PMIE), vascular disease, neurological infections, and osteomyelitis (Masters et al. (2019)). P. aeruginosa biofilms can cause severe pulmonary infections in patients with cystic fibrosis (Rabin et al. (2015). Biofilm formation mechanisms and targets for developing antibiofilm agents. Future Med. Chem. 7, 493-512). Haemophilus influenza biofilm is among the causative agents of otitis media (Akyildiz et al., (2013). Bacterial biofilm formation in the middle-ear mucosa of chronic otitis media patients. Indian J. Otolaryngol. Head Neck Surg. 65, 557-561). Periodontitis, an infection of the gums that damages the soft tissues as well as bones supporting the teeth, is normally caused by the biofilms of Pseudomonas aerobicus and Fusobacterium nucleatum (Jamal et al. (2018). Bacterial biofilm and associated infections. J. Chin. Med. Assoc. 81, 7-11), and chronic dental infections have been linked to carcinogenesis in the heart and brain. (Virtanen et al. (2014), History of Dental Infections Associates with Cancer in Periodontally Healthy Subjects: A 24-Year Follow-Up Study from Sweden. J Cancer. 5(2): 79-85). Furthermore, certain types of cancer have been associated with chronic infections, including for example gastric cancer (Helicobacter pylori) and cervical cancer (Chlamydia trachomatis). P. aeruginosa biofilm is also usually formed on chronic wounds (Rabin et al., 2015), and Chronic osteomyelitis is a biofilm infection, where microorganisms adhere to dead bone (Zimmerli and Sendi (2017). Orthopaedic biofilm infections. APMIS 125, 353-364). Equine fertility is also affected by P. aeruginosa biofilms which form on the endometrium and compromise uterine defense systems in reproducing mares, causing persistent mating-induced endometritis (PMIE), otherwise colloquially known as ‘Dirty Mare Syndrome’ (Satue and Gardon (2016), Infection and Infertility in Mares, Atef M. Darwish, ed. Genital Infections and Infertility (London: IntechOpen, 2016)).

It is believed that biofilm-related organisms account for over 80% of all microbial infections and exhibit high resistance to antimicrobial agents and components of the host defense system (both innate and adaptive) (Gulati and Nobile (2016), Candida albicans biofilms: development, regulation, and molecular mechanisms. Microbes Infect. 18(5):310-321). Despite long-term therapy with antibiotics for which bacteria show in vitro susceptibility, biofilm-growing microbials persist and destroy the infected tissue due to long-term inflammatory response, causing chronic infection (Hoiby et al., ESCMID guideline for the diagnosis and treatment of biofilm infections 2014. Clin Microbiol Infect 2015; 21 (Suppl 1): S1-25).

Recently, the impact of fungi and its role in chronic wound biofilms has begun to be appreciated (see, e.g., Ge & Wang, Current research on fungi in chronic wounds, Front Mol Biosci. 2022; 9: 1057766). As described by Ge and Wang, the fungal infection rate varies with different chronic wound types, but overall, the prevalence of fungi is extremely underestimated in the clinical treatment and management of chronic wounds. Wounds and ulcers can be colonized by host cutaneous, commensal or environmental fungi and evolve into local infections, causing fungemia as well as invasive fungal disease. Furthermore, the fungi involved in nonhealing wound-related infections help commensal bacteria resist antibiotics and the host immune response, forcing wounds to become reservoirs for multiresistant species, which are considered a potential key factor in the microbial bioburden of wounds and ulcers. Fungi can be recalcitrant to the healing process. Biofilm establishment is the predominant mechanism of fungal resistance or tolerance to antimicrobials in chronic nonhealing wounds. In March 2023, the United States Center for Disease Control released a press release titled “Increasing threat of spread of antimicrobial-resistant fungus in healthcare facilities”, specifically identifying Candida auris (C. auris), an emerging fungus considered an urgent antimicrobial resistance (AR) threat, as spreading at an alarming rate in U.S. healthcare facilities. Equally concerning was a tripling in 2021 of the number of cases that were resistant to echinocandins, the antifungal medicine most recommended for treatment of C. auris infections.

The increasing microbial resistance to present treatment regimens is due to an evolution in the genetic make-up of the microorganisms, enhancing their virulence and pathogenicity, and rapid emergence of drug-resistant species due to overuse of weakened doses of antibiotics, particularly as oriented in biofilms, and from the horizontal exchange of RNA and DNA among microbes within biofilms. Mechanisms attributed to treatment resistance in microbial biofilms include restricted penetration of antimicrobials through the biofilm matrix which can, in some cases, contribute to the antimicrobial tolerance of biofilms. Although biofilm matrices do not inhibit diffusion of antibiotics in general, restricted penetration of antibiotics through biofilms may occur in cases where the antibiotics bind to components of the biofilm matrix or the bacterial membranes such as extracellular DNA or when the antibiotics are inactivated by enzymes present in the matrix such as beta-lactam inactivation by beta-lactamases (see, e.g., Walters et al., Contributions of antibiotic penetration, oxygen limitation, and low metabolic activity to tolerance of Pseudomonas aeruginosa biofilms to ciprofloxacin and tobramycin. Antimicrob Agents Chemother 2003; 47:317-23, Chiang et al., Extracellular DNA shields against aminoglycosides in Pseudomonas aeruginosa biofilms. Antimicrob Agents Chemother 2013; 57:2352-61; Mulcahy et al., Extracellular DNA chelates cations and induces antibiotic resistance in Pseudomonas aeruginosa biofilms. PLoS Pathog 2008; 4:e1000213). In addition, efflux pumps may act to discharge antibiotics and antifungals from within the microbe to its exterior environment into the biofilm; there is evidence that such activity promotes biofilm formation. Almatar et al., Efflux pump inhibitors: new updates. Pharmacol Rep. 2021 February;73(1):1-16.

Differential physiological activity of the microbes in biofilms is another underlying cause of biofilm-associated antimicrobial tolerance (Ciofu et al., Antibiotic treatment of biofilm infections. APMIS 125:304-319 (2017)). Studies of Pseudomonas aeruginosa biofilms have provided evidence that the metabolic activity of the bacteria is high in the outer part of the biofilm whereas it is low in the inner part of the biofilm (Bagge et al., Dynamics and spatial distribution of beta-lactamase expression in Pseudomonas aeruginosa biofilms. Antimicrob Agents Chemother 2004; 48:1168-74; Werner et al. Stratified growth in Pseudomonas aeruginosa biofilms. Appl Environ Microbiol 2004; 70:6188-96). The available evidence suggests that the differential physiological activity seen in biofilms is caused by limited oxygen and nutrient penetration through the biofilm due to internal consumption (Ciofu 2017). Because many antibiotics target processes that occur in growing bacteria (e.g. replication, cell wall synthesis), biofilms containing cells with low metabolic activity display increased antimicrobial tolerance to these kinds of antibiotics (Walters et al., Contributions of Antibiotic Penetration, Oxygen Limitation, and Low Metabolic Activity to Tolerance of Pseudomonas aeruginosa Biofilms to Ciprofloxacin and Tobramycin. Antimicrob Agents Chemother. 2003; 47(1):317-323). In addition to antimicrobial tolerance, increased mutational load can significantly compromise the antimicrobial susceptibility of microbial biofilms (Poole, Bacterial stress responses as determinants of antimicrobial resistance, Journal of Antimicrobial Chemotherapy, Volume 67, Issue 9, September 2012, Pages 2069-2089).

Additional mechanisms of biofilm treatment resistance include differential expression of specific genes in biofilms, for example, the ndvB gene in P. aeruginosa which encodes an enzyme that is involved in the synthesis of periplasmic glucans that binds tobramycin and prevents cell death by sequestering the antibiotic. Slowly dividing or non-dividing bacteria (so called “persister” cells) that show diminished susceptibility to antibiotics contribute to the resistance mechanisms and antimicrobial tolerance in biofilms (Brooun et al., A dose-response study of antibiotic resistance in Pseudomonas aeruginosa biofilms. Antimicrob Agents Chemother 2000; 44:640-6; De Groote et al. Novel persistence genes in Pseudomonas aeruginosa identified by high-throughput screening. FEMS Microbiol Lett 2009; 297:73-9; Lewis K. Persister cells. Annu Rev Microbiol 2010; 64:357-72). Persister cells are believed to be the result of differentiation into a dormant state. The reduced metabolism exhibited by persister cells evidently enables them to escape the activity of antibiotics and antifungals that target fundamental cellular processes.

A number of strategies have evolved to treat chronic infection caused by microbial biofilms. One strategy is to provide high antibiotic concentration directly to the biofilm through topical administration. Topical administration provides high local concentrations by delivering antibiotics directly to the site of infection with lower or even undetectable serum concentrations, avoiding systemic side effects. Other strategies include the administration of sequential or combinatorial antimicrobial agents; the use of adjuvants to increase antimicrobial effects; the use of enzymes to temporarily disable the signalizing molexules (Quorum Sensing) that communicate and regulate the mode of life within the biofilm; and, the use of EPS degradation products to disrupt the biofilm matrix (Jackobsen et al., Bacterial Biofilm Control by Perturbation of Bacterial Signaling Processes. Int J Mol Sci. 2017 Sep. 13; 18(9):1970).

Nonetheless, even with recent advances in the understanding of the physiology of biofilms and the availability of novel treatments, given the difficulty in treating them, biofilms still account for, for example, over 60% of chronic skin infections, and remain major causes of chronic conditions including chronic wounds, hidradenitis suppurativa, atopic dermatitis, candidiasis, acne, onchomycosis, miliaria, impetiginized skin, pemphigus foliaceus, rosacea, chronic otitis media, and chronic otitis externa.

Although various novel treatment options are available, these show varying degrees of efficacy and the eradication of biofilms, including polymicrobial biofilms, in diseases still remains enigmatic. This lack of efficacy is especially troubling in light of the increased incidence of biofilm associated infections. Accordingly, there remains a strong need for safe and effective compounds and compositions to treat a range of microbial biofilms composed of mixed pathogens. Furthermore, there remains a need for compounds and compositions useful as coatings and additives that limit biofilm formation and subsequent infection, including on medical devices. Finally, there remains a need for compounds and compositions suitable for use eliminating or reducing microbial pathogens ability to attach to surfaces, thus inhibiting the development of biofilms.

### SUMMARY OF THE INVENTION

Provided herein are advantageous compositions comprising an ammonium silane, new ammonium silanes, methods of using ammonium silanes, and methods of synthesis thereof. In certain embodiments the ammonium silane is halide counterion free.

A key functional aspect described herein is the control of the ammonium counterion. It has been discovered that when halidic counterions such as Cl— are present in the solution, the rate of disadvantageous oligomerization and polymerization and subsequent precipitation of insoluble materials of the ammonium silane in aqueous media is increased. In the absence of chloride or other halidic counterions, ammonium silanes described herein are much more stable in water and the rate of disadvantageous polymerization/precipitation is much slower. It has been discovered that avoiding halidic counterions is beneficial for the water-stability of the active compounds. Hence, provided herein are new ammonium silanes including counterions that do not promote or catalyze undesired oligomerization/polymerization of the siloxane.

It has also been discovered that ammonium silanes can be stabilized by being mixed with an appropriate excipient (for example an excipient of Formula A below) to form an advantageous pharmaceutical composition. In certain embodiments the ammonium silane in the pharmaceutical composition is a non-halogen counterion salt. In other embodiments the ammonium silane in the pharmaceutical composition is a halogen counterion salt. In certain embodiments, an ammonium silane described herein is a charge balanced zwitterion and A− or A2⁻ is absent, as described further below.

Thus, in certain aspects provided herein is a pharmaceutical composition comprising an excipient selected from Formula A and a compound of Formula B, wherein:
Formula A is selected from:

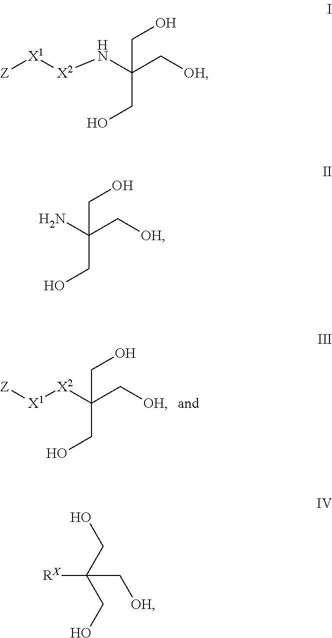

or a pharmaceutically acceptable salt thereof;
Formula B is selected from:

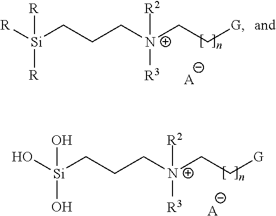

or a pharmaceutically acceptable salt thereof,
each R is independently selected from the group consisting of OH, alkoxy for example OC₁-C₆alkyl, and Cl;
R^{X} is selected from:

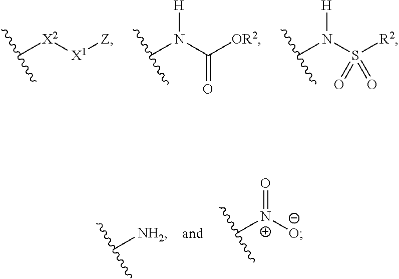

G is hydrogen, alkyl, aryl, cycloalkyl, heterocycle, heteroaryl, —CN, or CF₃;
each R² is selected from: hydrogen, C₁-C₆ alkyl, —O;
R³ is selected from: hydrogen, C₁-C₆ alkyl, —O;
X¹ is selected from the group consisting of a bond,

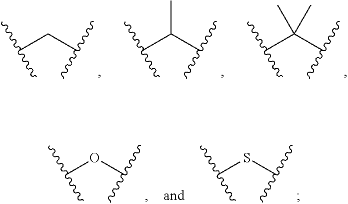

X² is selected from the group consisting of a bond,

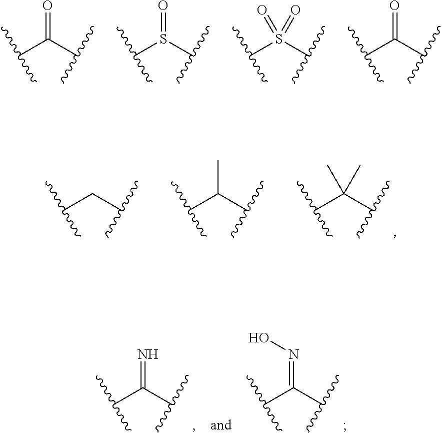

Z is selected from: alkyl, —NO₂, —SO₃H, —SO₂H, —SO₃⁻, —SO₃M, —CO₂H, —CO₂⁻, —CO₂M, phosphate, OH, NH₂, and SH;
A⁻ is an anion;
M is a cation, for example sodium, lithium, or potassium; and
each n is independently selected from the group consisting of 1, 2, 3, 4, 5, 6, 7, 8, 9, 10, 11, 12, 13, 14, 15, 16, 17, and 18.

In certain embodiments Formula A is

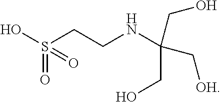 [SMILES: O=S(=O)(O)CCNC(CO)(CO)CO]

In certain embodiments Formula A is

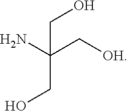

In certain embodiments Formula B is

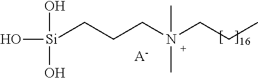

The excipient of Formula A and the compound of Formula B can be in any ratio in the composition that provides the desired medicinal properties. For example, in certain embodiments there is about 1.25, 1.5, 1.75, 2, 2.25, 2.5, 2.75, 3, 3.25, 3.5, 3.75, or 4 molecules of Formula A for every one molecule of Formula B. In certain embodiments there is at least about 1.25, 1.5, 1.75, 2, 2.25, 2.5, 2.75, 3, 3.25, 3.5, 3.75, or 4 molecules of Formula A for every one molecule of Formula B.

In certain aspects a composition is provided comprising an excipient selected from Formula H and a compound of Formula B, wherein:
Formula H is selected from:

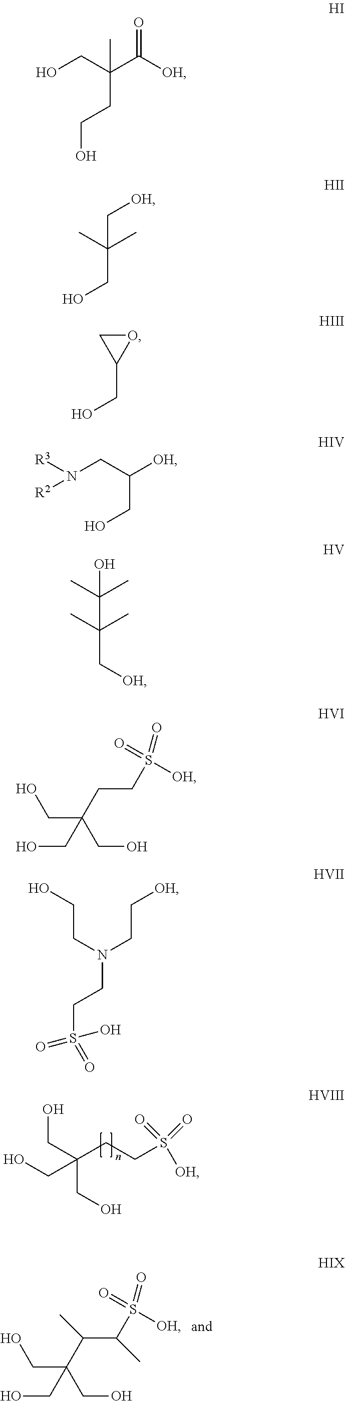

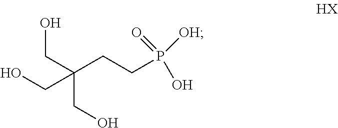 [SMILES: O=P(O)(O)CCC(CO)(CO)CO]

or a pharmaceutically acceptable salt thereof.
or in an alternative embodiment Formula H is selected from

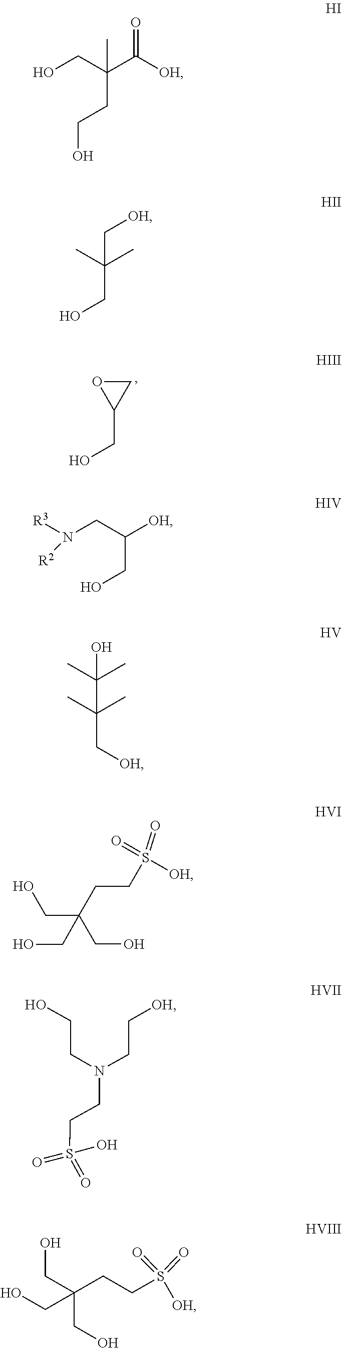

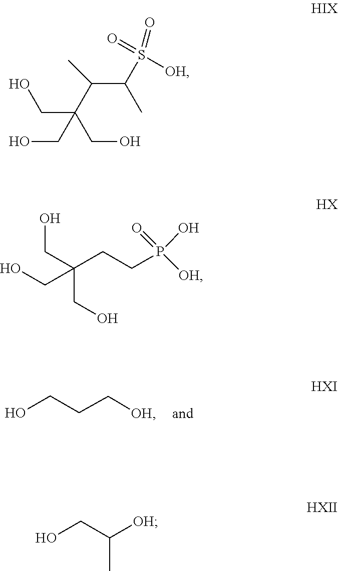

or a pharmaceutically acceptable salt thereof.

The excipient of Formula H and the compound of Formula B can be in any ratio in the composition that provides the desired properties. For example, in certain embodiments there is about 1.25, 1.5, 1.75, 2, 2.25, 2.5, 2.75, 3, 3.25, 3.5, 3.75, or 4 molecules of Formula H for every one molecule of Formula B. In certain embodiments there is at least about 1.25, 1.5, 1.75, 2, 2.25, 2.5, 2.75, 3, 3.25, 3.5, 3.75, or 4 molecules of Formula H for every one molecule of Formula B.

In some embodiments, A⁻ is a stabilized anion selected from:

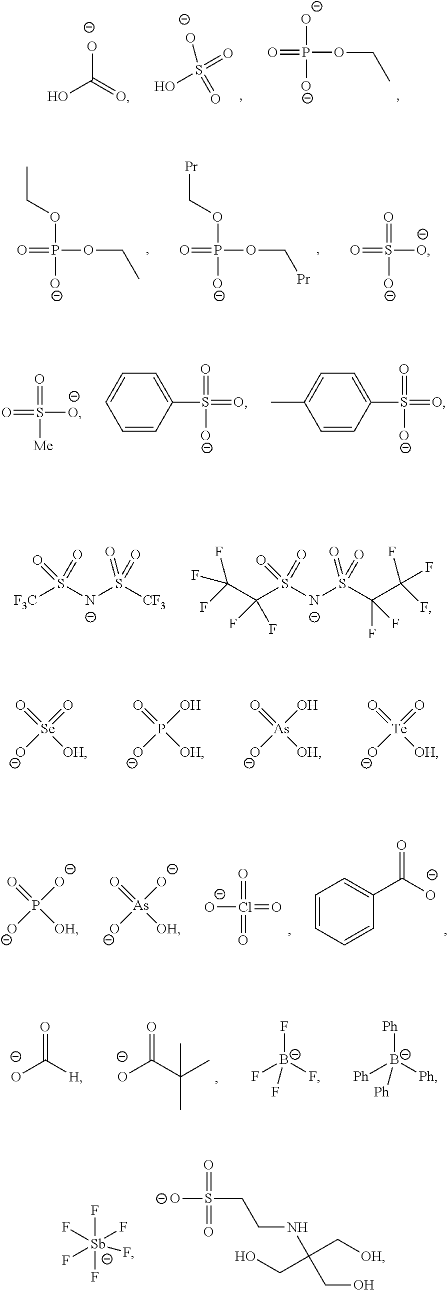

chlorite, perchlorate, hydroxide, formate, acetate, lactate, benzoate, nitrate, and salicylate anion.

In some embodiments, A⁻ is a bulky anion for example a bulky anion selected from:

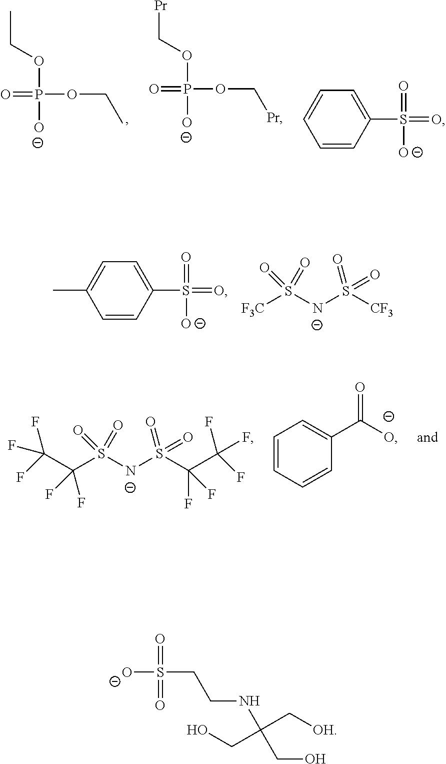

In other embodiments A⁻ is chloride, fluoride, iodide, or bromide.

In certain embodiments, the balancing anion is chloride or hydroxide.

In certain aspects an ammonium silane described herein is selected from Formula C and Formula Hc:

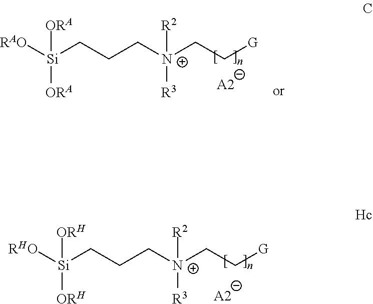

R^{A} is selected from:

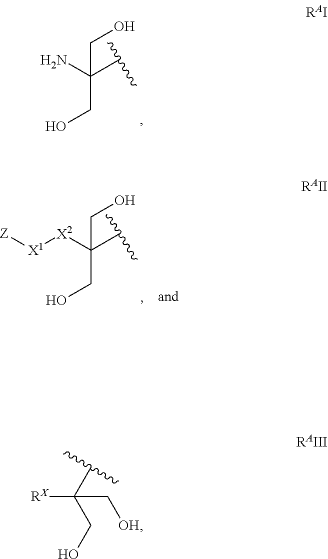

R^{H} is selected from:

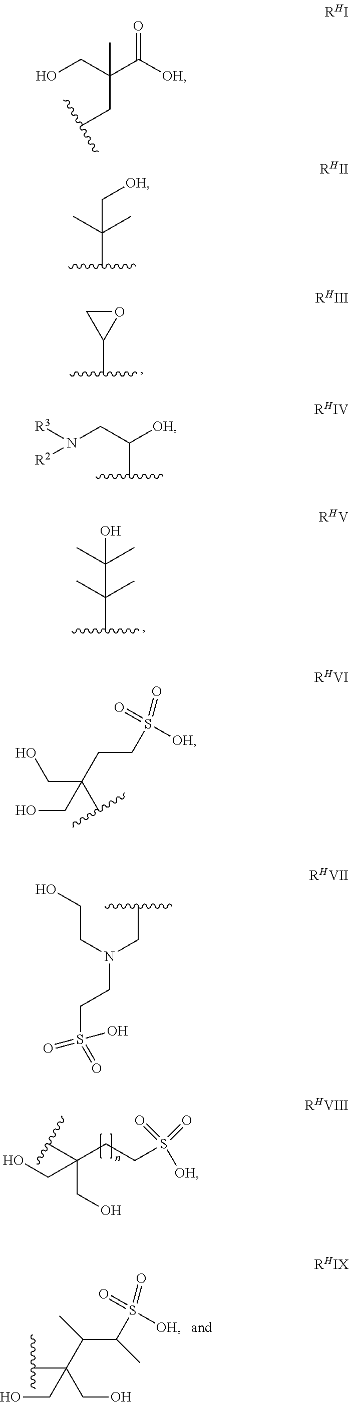

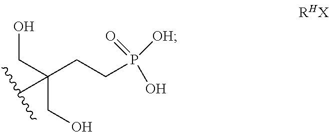 [SMILES: *C(CO)(CO)CCP(=O)(O)O]

or a pharmaceutically acceptable salt thereof;
or in an alternative embodiment R^{H} is selected from:
R^{H} is selected from:

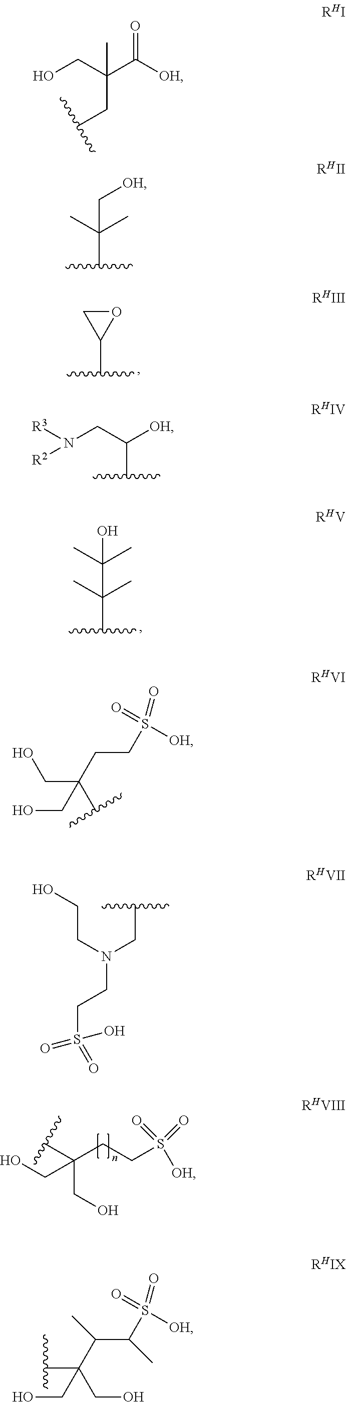

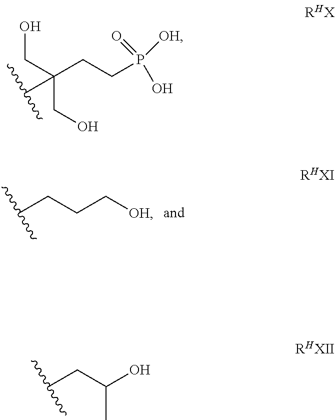

or a pharmaceutically acceptable salt thereof;
A2⁻ is a non halogen anion or is absent if the compound of Formula C or Formula He is a charge balanced zwitterion.

Non-limiting examples of A2 include

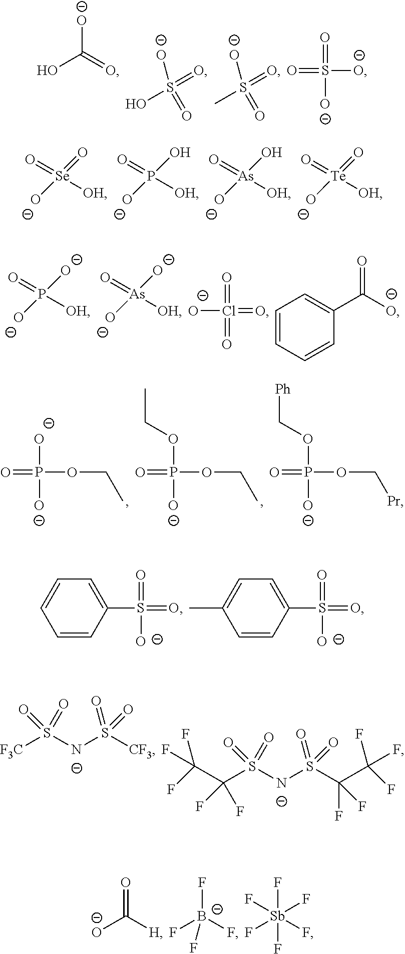

chlorite, perchlorate, hydroxide, formate, acetate, lactate, benzoate, nitrate, and salicylate anion.

In some embodiments, A2⁻ is a bulky anion for example a bulky anion selected from:

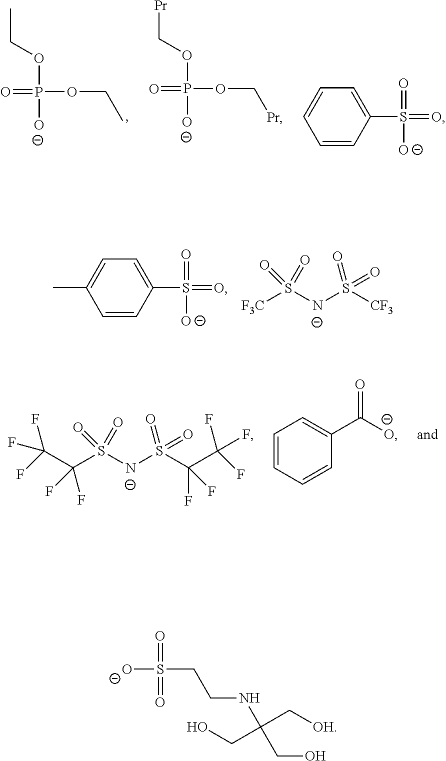

In certain aspects a compound is provided of Formula C wherein Formula C is selected from:

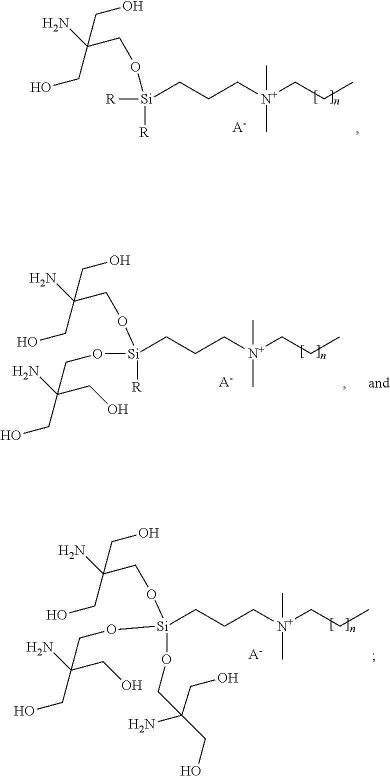

or a pharmaceutically acceptable salt thereof.

In certain embodiments the compound of Formula C is:

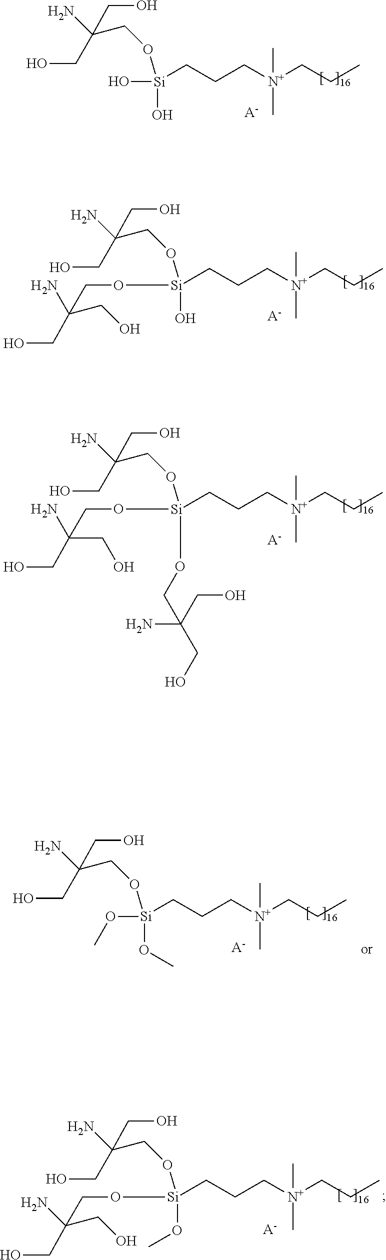

or a pharmaceutically acceptable salt thereof.

In certain embodiments the compound of Formula C is

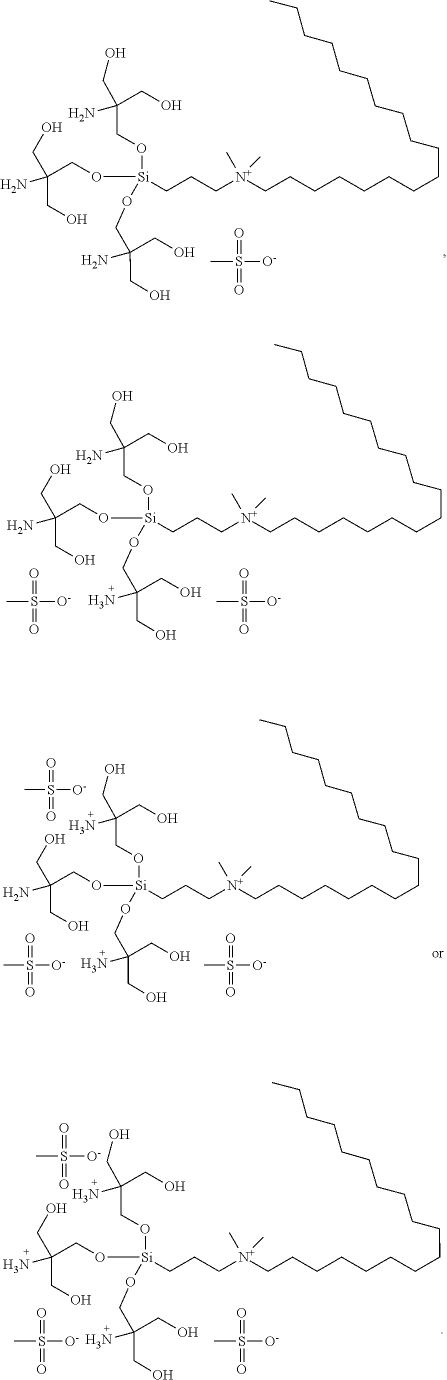

In alternative embodiments the compound of Formula C is

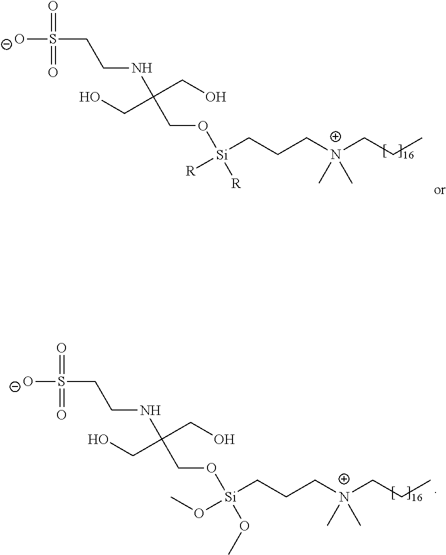

In other embodiments, the compound of Formula C is

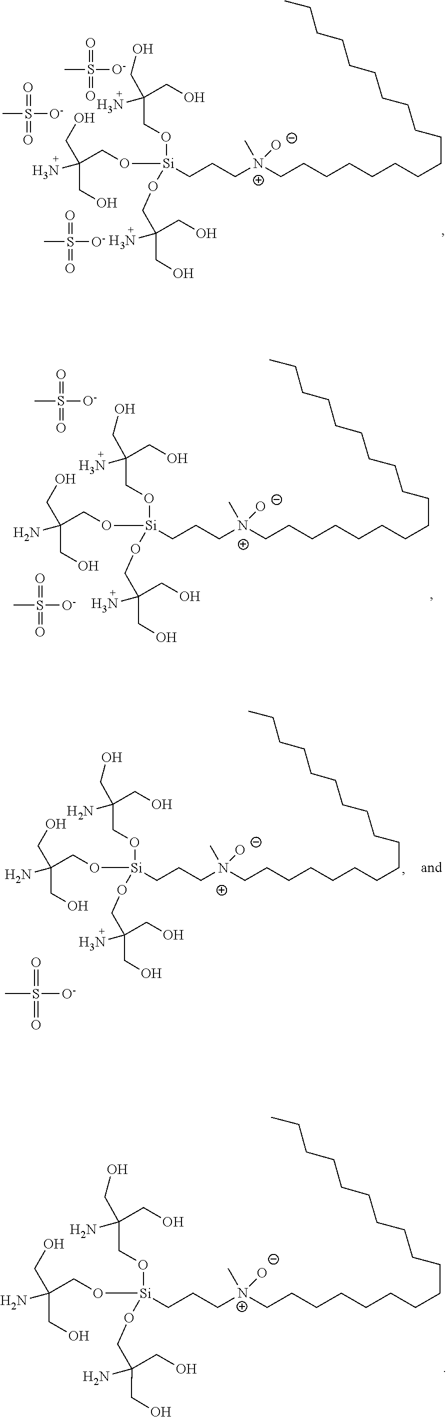

In other embodiments, the compound of Formula C is selected from:

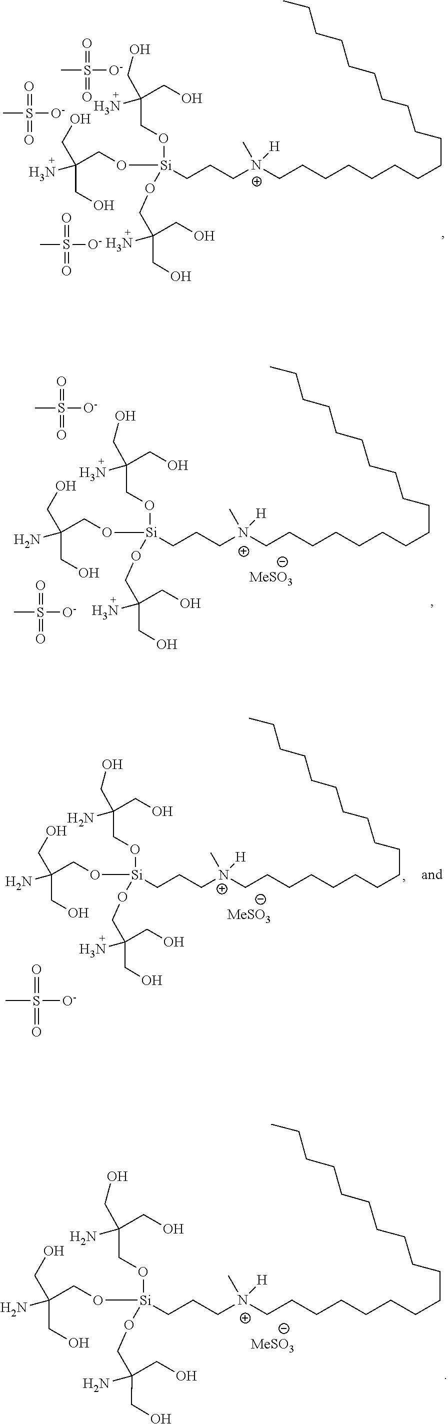

In certain embodiments the compound of Formula C is selected from:

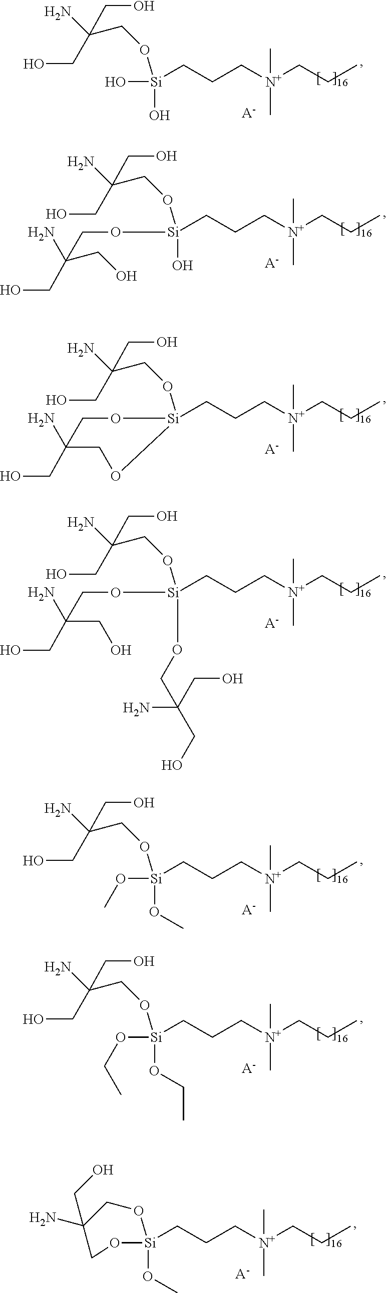

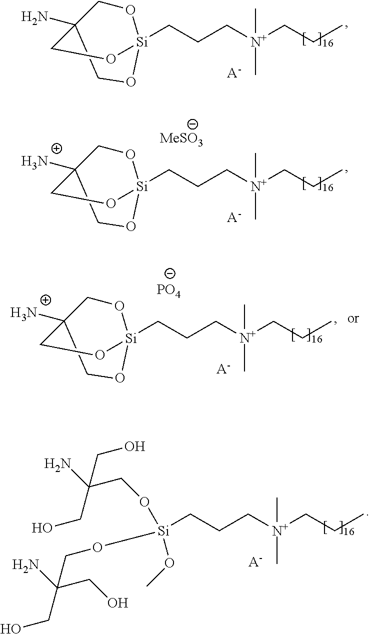

Further described herein are synthetic methods for the synthesis of an ammonium silane with a non-halidic counterion. It has been discovered that by using an organic solvent and a base selected from MeONa, sodium mesylate, NaH₂PO₄, NaHSO₄, or another non-halide, for example a counter ion selected from A2, that sodium chloride can be precipitated from solution providing a halide-free ammonium silane. The resulting ammonium silane with a non-halidic counterion has advantageous properties over those currently known in the art possessing halide counterions, for example improved stability in the presence of water. Non-limiting examples of these synthetic methods resulting in an ammonium silane described herein are provided.

In some embodiments, a base promotes the reaction. For example:

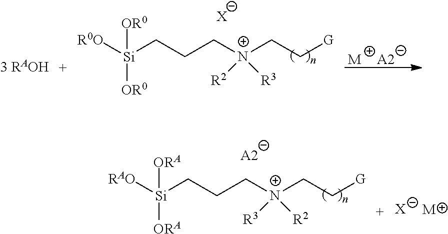

wherein M⁺ is a metal, X⁻ is a halide, and all other variables are as defined herein.

In other embodiments, an acid promotes the reaction. In these embodiments, the balancing anion for the ammonium is the conjugate base of the acidic reagent.

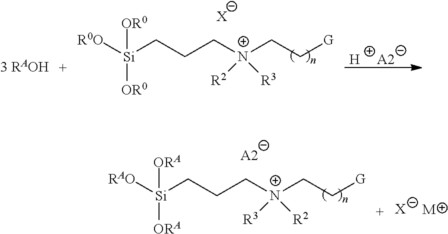

and all other variables are as defined herein.

This synthetic method can also be used on trihydroxysilyl ammonium chlorides to prepare halide free trihydroxysilyl ammonium compounds.

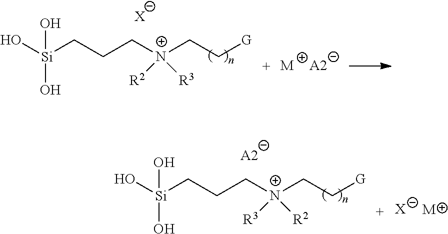

In certain aspects a compound described herein can be used to generate a halide-free sol-gel. For example, by mixing the halide-free compound described herein with an alkoxy or hydroxy compound, including alkoxy or hydroxy silane. Non-limiting examples of alkoxy and hydroxy compounds, including alkoxy or hydroxy silanes, include Ti(OH)₄, Al(OH)₃, Al(OH)₄⁻, P(OH)₄, Se(OH)₄, B(OH)₃, Sn(OH)₄, Te(OH)₄, Mg(OH)₂, Zn(OH)₂, B(OH)₃, B(OH)₄⁻, Ti(Oalkyl)₄, Al(Oalkyl)₃, Al(Oalkyl)₄⁻, P(Oalkyl)₄, Se(Oalkyl)₄, B(Oalkyl)₃, Sn(Oalkyl)₄, Te(Oalkyl)₄, Mg(Oalkyl)₂, Zn(Oalkyl)₂, B(Oalkyl)₃, and B(Oalkyl)₄⁻. In certain embodiments the alkoxy compound is silane, such as Si(OEt)₄.

Non-limiting examples of halid-free sol-gels of the present invention include:

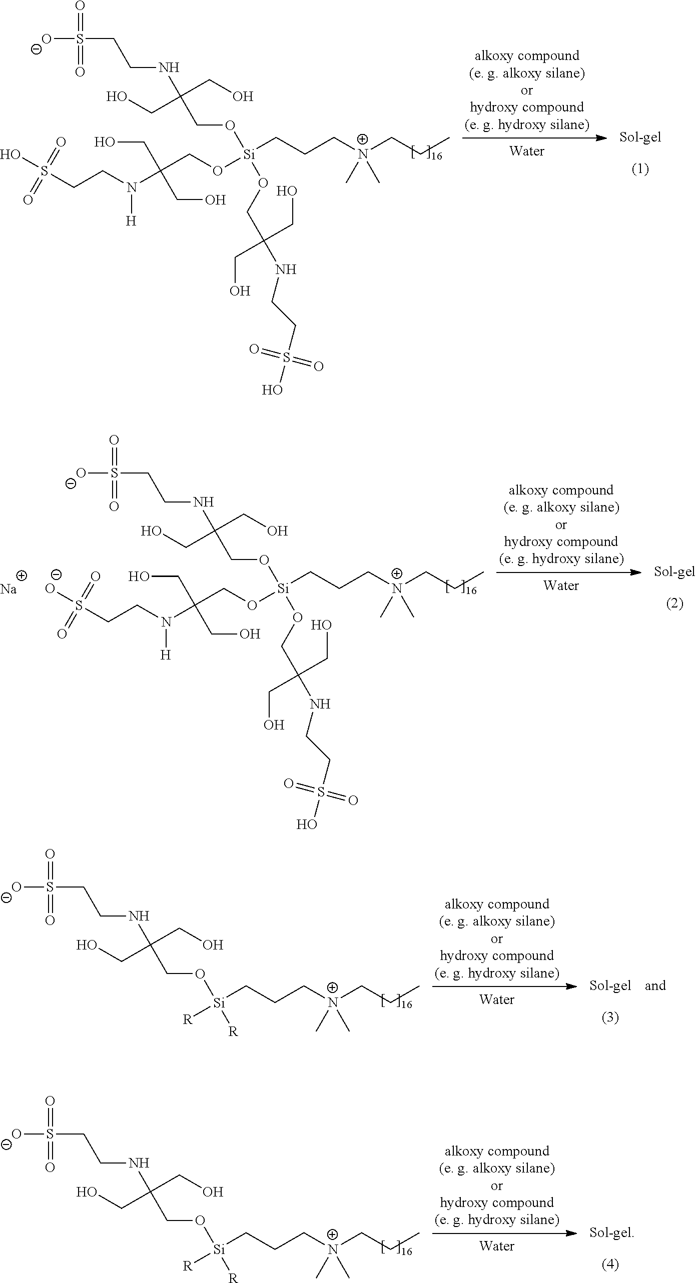

In some embodiments, an ammonium silane described herein includes a substituent that has a negative charge. The substituent with the negative charge may be balanced with a cation such as sodium or potassium.

In some embodiments, an ammonium silane is provided as a zwitterion, in which the positive charge of the internal quaternary ammonium N is balanced with an anionic functional group covalently attached to a substituent in the molecule.

Also provided herein are compositions thereof in the form of a powder, lyophilized powder, or otherwise solid stable storage form.

The ammonium silanes and pharmaceutical compositions described herein can be used in a number of applications to inhibit or prevent the growth of microbes, including microbial biofilms. In one aspect, an ammonium silane or pharmaceutical composition described herein can be administered in an effective amount to treat a range of human and animal infections, including bacterial infections including Gram-positive bacteria and/or Gram-negative bacteria, fungal infections, viral infections, amoeba infections, and combinations thereof, for example polymicrobial infections The fungal infection may comprise a yeast, a mold, or a combination of thereof. The ammonium silane and compositions described herein can treat microorganisms in a biofilm, including a biofilm comprising mixed microbes.

Accordingly, in some embodiments, a pharmaceutical composition comprising an ammonium silane described herein can be used in an effective amount in a topical formulation or wash for direct application to an infection or wound, or incorporated into, for example, a dressing, bandage, surgical packing, gauze, wrap, ointment, gel, conformable foam, wash, or film for application to a wound, for example, a chronic wound or burn. When incorporated into an article, an ammonium silane described herein may be incorporated in a manner such that the article provides controlled release of the compound or its pharmaceutically acceptable salt or composition into the surrounding area to provide extended inhibition of microbial growth.

In some embodiments, a topical infection in a human or animal can be treated with an ammonium silane or pharmaceutical composition described herein, wherein the infection comprises a microbe such as, for example, Acanthamoeba, Acinetobacter (Gram-negative), Pseudomonas (Gram-negative), Proteus (genus of Gram-negative Proteobacteria), Staphylococcus (Gram-positive), Streptococcus (Gram-positive), MRSA (methicillin resistant S. aureus), Escherichia coli (Gram-negative), Propionibacterium (Gram-positive), Klebsiella (Gram-negative), Enterococcus (Gram-positive), Haemophilius influenzae, and fungi such as Fusarium, Aspergillus, Cladosporium, Curvularia and Candida, and dermatophytes such as Trichophyton, Microsporum, and Epidermophyton, or combinations or biofilms comprising combinations thereof. In particular embodiments, the topical infection comprises a Candida fungal infection, for example, but not limited to, C. albicans, C. auris, or C. glabrata, or a combination thereof. In some embodiments, the infection is a mixed infection that includes bacterial species, fungal species, and viral species.

In some embodiments, an effective amount of an ammonium silane or pharmaceutical composition described herein is used to treat a chronic wound, for example, a pressure ulcer, venous ulcer, arterial wounds, neuropathic ulcer, diabetic ulcer, for example a lower limbic ulcer or foot ulcer, skin tear, or moisture-associated skin damage (MASD), for example incontinence-associated dermatitis. In some embodiments, an ammonium silane or pharmaceutical composition described herein are used to treat a wound caused by a burn.

In some embodiments, an ammonium silane or pharmaceutical composition described herein are used in an effective amount to treat, prevent or eliminate an ocular infection in a host in need thereof. In some embodiments, the ocular infection is corneal keratitis. In another embodiment, the ocular infection is bacterial keratitis, for example keratitis caused by Staphylococcus aureus or Pseudomonas aeruginosa. In another embodiment, the ocular infection is fungal keratitis, for example keratitis caused by Fusarium spp., Aspergillus spp., Candida spp., or Curvularia spp. In some embodiments, the ocular infection is Acanthamoebic keratitis. In another embodiment, the ocular infection is viral keratitis, for example Herpes simplex virus (HSV) keratitis. In another embodiment, the ocular infection is bacterial conjunctivitis, for example conjunctivitis caused by Staphylococcus aureus, Haemophilus influenzae, Streptococcus pneumoniae or Pseudomonas aeruginosa. In another embodiment, the ocular infection is viral conjunctivitis, for example conjunctivitis caused by adenovirus or enterovirus.

In another embodiment, the ocular infection is polymicrobial, which is more difficult to diagnose and treat. For example, when Acanthamoebic keratitis is associated with bacteria there is an increased risk of vascularization and prolonged healing. In another embodiment, the ocular infection is sequential with one or more opportunistic organisms that can also cause infection. For example, a herpetic corneal ulcer can provide a niche for establishment of bacterial or fungal pathogens.

In another aspect, an ocular composition is provided comprising an affective amount of ammonium silane or pharmaceutical composition described herein, and an acceptable carrier for the eye. In some embodiments, the ocular composition does not contain any byproducts or additives, for example an alcohol. In some embodiments, the ocular composition is substantially free of methanol.

In some embodiments, the infection to be treated is an infection of the tongue (thrush), gum (gingivitis), supragingival plaque, periodontitis, dental abscess, facial cellulitis, tooth decay, or plaque. In some embodiments, ammonium silane or pharmaceutical composition described herein is provided in a mouth rinse to treat oral infections and conditions. In some embodiments, an ammonium silane described herein is incorporated into a dental appliance, such as a toothbrush, gloss, or tooth pick.

In some embodiments, a method is provided for the treatment of an ear infection in a host in need thereof comprising administering an effective amount of ammonium silane or pharmaceutical composition described herein. In some embodiments, the ear infection is an inner ear infection (otitis interna). In some embodiments, the ear infection is an outer ear infection (otitis externa). In another embodiment, the ear infection is a middle ear infection (otitis media). In some embodiments, the ear infection is caused by a bacterium or a fungi. In another embodiment, the ear infection is caused by both a bacterium and a fungi. In another embodiment, the ear infection is caused by a biofilm which may contain a combination of bacterial and fungal cells. In another embodiment, the ear infection is caused by a biofilm which may contain a combination of bacterial and fungal cells, and one or more viruses.

In some embodiments, the infection to be treated is an infection of the nails, for example a fungal infection of the nails. In some embodiments, the nail infection is onychomycosis. In another embodiment, a formulation for the treatment of onychomycosis in a host in need thereof is provided comprising administering an effective amount of an ammonium silane described herein, in a carrier suitable for delivery to the nail bed. In some embodiments, the carrier is dimethylsulfoxide.

In another embodiment, a method is provided for the treatment or prevention of a vaginal infection in a host in need thereof, comprising administering an effective amount of ammonium silane or pharmaceutical composition described herein. In some embodiments, the vaginal infection is vulvovaginal candidiasis. In some embodiments, the vaginal infection, for example vulvovaginal candidiasis is a fungal infection caused by Candida spp. In some embodiments, the vaginal infection is bacterial vaginosis. In some embodiments, the vaginal infection, for example vulvovaginal candidiasis is a bacterial infection caused by Lactobacilli, Bacteroides, Peptostreptococcus, Fusobacterium, and/or Eubacterium.

In some embodiments, the infection is a uterine infection, for example endometritis. In some embodiments, the uternine infection is a bacterial infection caused by microorganisms selected from Streptococcus zooepidemicus, hemolytic Escherichia coli, Staphylococcus aureus, Candida, Aspergillus, or a combination thereof. In some embodiments, the uterine infection is an equine uternine infection. In some embodiments, the condition to be treated by the compounds described herein is persistent mating-induced endometritis (PMIE).

In one aspect, a method is provided for the treatment of a skin disorder in a host in need thereof comprising administering an effective amount of ammonium silane or pharmaceutical composition described herein. In certain embodiments, the skin disorder is for example, acne vulgaris, cystic acne, eczema, folliculitis, or a skin infection. In certain embodiments, the skin disorder, for example, acne vulgaris is caused by gram positive bacterium Propionibacterium acnes, and/or Staphylococcus epidermidis. In certain embodiments, the skin disorder is, for example, eczema (atopic dermatitis), eczema herpeticum, eczema vaccinatum, or eczema coxsackium and is caused by a bacterial and/or viral infection. In certain embodiments, the skin disorder is, for example, eczema (atopic dermatitis), and is caused by bacteria, for example, staphylococcal bacteria such as Staphylococcus aureus or streptococcal bacteria. In certain embodiments, the skin disorder is, for example, eczema (atopic dermatitis), and is caused by a virus, for example herpes simplex virus, and/or Molluscum contagiosum. In certain embodiments, the skin disorder is, for example, caused by Staphylococcus aureus (S. aureus).

In certain aspects, an ammonium silane described herein is provided as a pharmaceutical composition comprising a pharmaceutically acceptable carrier. In certain embodiments, the pharmaceutically acceptable carrier comprises an aqueous or glycerin solution, for example, water, saline, or phosphate buffered saline. In certain embodiments, the glycerin solution is selected from glycerol, glycidol, glycerol-propylene oxide copolymer, ethylene glycol, propylene glycol, polyethylene glycol, or polypropylene glycol, or combinations thereof. In some embodiments, the pharmaceutically acceptable carrier is calcium phosphate. In some embodiments, an ammonium silane described herein can be provided as a stable powder or lyophilized material that can be formulated before administration using a pharmaceutically acceptable carrier.

In another aspect, a kit is provided comprising a vial which contains a sterile aqueous solution and a vial comprising an ammonium silane as described herein and an application device. In some embodiments, the application device is a syringe. In some embodiments, the application device is a balloon-tipped catheter.

In another aspect, a kit is provided comprising a sterile aqueous solution and an ammonium silane as described herein, with one or more compounds selected from solketal, epichlorohydrin, and polyvinyl alcohol.

In an alternative embodiment, ammonium silane or pharmaceutical composition described herein can be incorporated into a dressing, conformable foam, putty, or polymer for use in dressings, bandages, or films for use in medical applications, for example in a wound dressing or in surgical packings to reduce the risk of infection. In yet another alternative embodiment, a new organosilane described herein can be incorporated into a medical device or implant, for example but not limited to an orthopedic or dental implant.

In another embodiment, a method is provided for the treatment of a biofilm or microbial contamination on an abiotic (i.e., inanimate) surface comprising applying to the surface an effective amount of one or more ammonium silanes described herein, or compositions thereof. In certain embodiments, the abiotic (i.e., inanimate) surface is treated directly with a solution containing one or more ammonium silanes described herein, or compositions thereof to remove and/or prevent the recurrence of a bacterial biofilm or bacterial contamination. In some embodiments, the viral biofilm or viral contamination is caused by SARS Coronavirus 2 (COVID-19).

In additional aspects, an ammonium silane described herein or composition thereof can be used in an effective amount as an additive incorporated into materials, such as decellularized tissue in sheet, particle, or powdered form, which can be used, for example, in an artificial organ, joint and bone repair, and tissue regeneration. In additional aspects, an ammonium silane described herein or composition thereof can be incorporated in an effective amount as additives incorporated into or on medical devices to inhibit the growth and reduce/eliminate the initial attachment and subsequent colonization of the device and any surrounding tissue.

In some embodiments, an ammonium silane described herein or composition thereof can be used in an effective amount as an additive incorporated into materials for example, fly ash, resins, acrylics, acetates, resins that are melamine or phenolic, siliceous, polyester, polyurethane, polyacrylic, polycarbonate, or thermoplastic polymers, to provide or enhance antimicrobial, adhesive and strengthening properties of abiotic products.

In some embodiments provided herein is a compound as described herein, or an acceptable composition thereof; useful in an effective amount as a preservative for food products, beverages, pharmaceuticals, paints, biological samples, cosmetics, wood, concrete and other such products, to prevent decomposition or spoilage by biofilms or microbial contamination.

Thus, provided herein are compounds and composition which include one or more of the following features:
(a) an ammonium silane of Formula C or Formula Hc;
(b) a pharmaceutical composition comprising an excipient of Formula A or Formula H and a compound of Formula B or a pharmaceutically acceptable salt thereof,
(c) use of (a) or (b), in the manufacture of a medicament for the treatment of a bacterial, fungal and/or viral infection;
(d) a method for manufacturing a medicament intended for the therapeutic use of treating a bacterial, fungal and/or viral infection, characterized in that (a) or (b) is used in the manufacture;
(e) a method for treating a bacterial, fungal and/or viral infection, comprising administering an effective amount of (a) or (b), to a host in need thereof;
(f) a method for treating a bacterial, fungal and/or viral infection, comprising administering an effective amount of (a) or (b), to a host in need thereof;
(g) an antimicrobial composition comprising an effective amount of one or more ammonium silanes of Formula C or Formula Hc, and a pharmaceutically acceptable excipient;
(h) a powder or solidified formulation, including a lyophilized powder formulation, comprising one or more ammonium silanes Formula C or Formula Hc;
i) a kit comprising a powder or solid formulation, including a lyophilized powder, of one or more ammonium silanes of Formula C or Formula Hc, a sterile aqueous solution, and an application device;
(j) a kit comprising a sterile aqueous solution comprising one or more ammonium silanes of Formula C or Formula Hc, and an application device;
(k) a process to synthesize an ammonium silane of Formula C or Formula Hc;
(l) an ammonium silane of Formula C or Formula Hc, as a mixture of enantiomers or diastereomers (as relevant), including as a racemate;
(m) an ammonium silane of Formula C or Formula Hc, in an enantiomerically or diastereomerically (as relevant) enriched form, including as an isolated enantiomer or disastereomer (i.e., greater than 85, 90, 95, 97 or 99% pure);
(n) a process for the preparation of therapeutic products that contain an effective amount of one or more ammonium silanes Formula C or Formula Hc;
(o) a product formed by reacting an ammonium silane of Formula C or Formula He with one or more compounds of glycerol, glycerol-propylene oxide copolymer, solketal, glycidol, or epichlorohydrin;
(p) a product formed by reacting an ammonium silane of Formula C or Formula He with one or more compounds of a glycerol-propylene oxide copolymer, solketal, glycidol, or epichlorohydrin for use in the treatment of a bacterial, fungal and/or viral infection;
(q) use of a product formed by reacting an ammonium silane of Formula C or Formula Hc, one or more compounds of glycerol, glycerol-propylene oxide copolymer, solketal, glycidol, or epichlorohydrin in the manufacture of a medicament for the treatment of a bacterial, fungal and/or viral infection;
(r) a method for manufacturing a medicament intended for the therapeutic use of treating a bacterial, fungal and/or viral infection, characterized in that an effective amount of a product formed by reacting a compound of Formula C or Formula He with one or more compounds of glycerol, a glycerol-propylene oxide copolymer, solketal, glycidol, or epichlorohydrin, is used for the manufacture;
(s) a method for treating a bacterial, fungal and/or viral infection, comprising administering an effective amount of a product formed by reacting an ammonium silane of Formula C or Formula He with one or more compounds of glycerol, a glycerol-propylene oxide copolymer, solketal, glycidol, or epichlorohydrin to a patient in need thereof;
(t) a method for treating a bacterial, fungal and/or viral infection, comprising administering an effective amount of a product formed by reacting an ammonium silane of Formula C or Formula He with one or more compounds of glycerol, a glycerol-propylene oxide copolymer, solketal, glycidol, or epichlorohydrin to a patient in need thereof;
(u) an antimicrobial composition comprising an effective amount of a product formed by reacting an ammonium silane of Formula C or Formula He with one or more compounds of glycerol, a glycerol-propylene oxide copolymer, solketal, glycidol, or epichlorohydrin and a pharmaceutically acceptable excipient;
(v) a powder formulation or solid formulation, including a lyophilized powder, comprising a product formed by reacting an ammonium silane of Formula C or Formula He with one or more compounds of glycerol, glycidol, glycerol-propylene oxide copolymer, ethylene glycol, propylene glycol, polyethylene glycol, polypropylene glycol, solketal, glycidol, or epichlorohydrin;
(w) a kit comprising a powder or solid formulation, including a lyophilized powder, of a product formed by reacting an ammonium silane of Formula C or Formula He with one or more compounds of glycerol, a glycerol-propylene oxide copolymer, solketal, glycidol, or epichlorohydrin, a sterile aqueous solution, and an application device;
(x) a kit comprising a sterile aqueous or glycerin solution comprising a product formed by reacting an ammonium silane of Formula C or Formula He with one or more compounds of glycerol, a glycerol-propylene oxide copolymer, solketal, glycidol, or epichlorohydrin; and an application device;
(y) a process for the preparation of therapeutic products formed by reacting an ammonium silane of Formula C or Formula He with one or more compounds of glycerol, glycidol, glycerol-propylene oxide copolymer, ethylene glycol, propylene glycol, polyethylene glycol, polypropylene glycol, solketal, glycidol, or epichlorohydrin;
(z) a dressing, bandage, surgical packing, film, wrap, comformable foam, or other type material incorporating a compound of Formula C or Formula Hc;
(aa) use of a dressing, bandage, surgical packing, film, wrap, comformable foam, or other type material incorporating a compound of Formula C or Formula He for treating a skin infection, wound, or ulcer, for example, but not limited to, a lower limb ulcer or a diabetic ulcer such as a diabetic lower limb ulcer or diabetic foot ulcer;
(bb) an oligomer or polymer product of Formula C or Formula Hc;
(cc) an oligomer or polymer product of Formula C or Formula He for use in the treatment of a bacterial, fungal and/or viral infection;
(dd) use of an oligomer or polymer product of Formula C or Formula He in an effective amount, in the manufacture of a medicament for the treatment of a bacterial, fungal and/or viral infection;
(ee) a method for manufacturing a medicament intended for the therapeutic use of treating a bacterial, fungal and/or viral infection, characterized in that an oligomer or polymer product of Formula C or Formula He is used in the manufacture;
(ff) a method for treating a bacterial, fungal and/or viral infection, comprising administering an effective amount of an oligomer or polymer product of Formula C or Formula Hc, to a host in need thereof;
(gg) an antimicrobial composition comprising an effective amount of a oligomer or polymer product of Formula C or Formula Hc, and a pharmaceutically acceptable excipient;
(hh) a powder or solid formulation, including a lyophilized powder, comprising an oligomer or polymer product of Formula C or Formula He or combinations thereof;
(ii) a kit comprising a powder or solid formulation, including a lyophilized powder, of an oligomer or polymer product of Formula C or Formula Hc, a sterile aqueous solution, and an application device;
(jj) a kit comprising a sterile aqueous or glycerin solution comprising an oligomer or polymer product of Formula C or Formula Hc; and an application device;
(kk) a process for the preparation of therapeutic products that contain an oligomer or polymer product of Formula C or Formula Hc;
(ll) a dressing, bandage, surgical packing, film, wrap, comformable foam, or other type material incorporating a compound of Formula C or Formula Hc;
(mm) use of a dressing, bandage, surgical packing, film, wrap, comformable foam, or other type material incorporating a compound of Formula C or Formula He for treating a skin infection, wound, or ulcer, for example, but not limited to, a lower limb ulcer or a diabetic ulcer such as a diabetic lower limb ulcer or diabetic foot ulcer; and,
(nn) a medical or dental implant or bone graft composition incorporating a compound of Formula C or Formula Hc.

### DETAILED DESCRIPTION OF THE INVENTION

Provided herein are advantageous pharmaceutical compositions comprising an ammonium silane, new ammonium silanes, methods of using ammonium silanes and methods of synthesis. In certain embodiments the ammonium silane described herein is halide free.

A key functional aspect described herein is the control of the counterion. It has been discovered that when halidic counterions such as Cl— are present in the solution, the rate of disadvantageous polymerization and precipitation of the ammonium silane in aqueous media is increased. In the absence of chloride or other halidic counterions, the ammonium silanes described herein are much more stable to water and the rate of disadvantageous polymerization is much slower. It has been discovered that avoiding halidic counterions is beneficial for the water-stability of the active compounds. Hence the present invention provides new ammonium silanes including non-reactive (non-catalytic) counterions.

An ammonium silane compound described herein has improved properties such as reduced polymerization and increased shelf life in water as compared to alkoxy silanes. Known alkoxy silane compounds react with water and other nucleophiles leading to exchange reactions at silicon and subsequent polymerization. (Osterholtz and Pohl J. Adhesion Sci. Technol. Vol. 6, No. 1, pp. 127-149, 1992). Overall, the exchange mechanism at silicon is complex and multi-factorial, depending on the nature of the alkoxy groups, pH, concentration of nucleophile, and other factors. Known trihydroxysilyl compounds are known to rapidly hydrolyze and polymerize. (Cypryk at. al. New J. Chem., 2019, 43, 15222-15232).

The inventors have discovered that the presence of a halide such as a chloride ion may play a role in the acceleration of the condensation of silanol to siloxane oligomers. The absence of a chloride counterion is related to increased water stability, decreased polymerization, and longer shelf-life of the ammonium silanes described herein.

It has also been discovered that ammonium silanes can be stabilized by being mixed with an appropriate excipient (for example an excipient of Formula A below) to form an advantageous pharmaceutical composition. In certain embodiments the ammonium silane in the pharmaceutical composition possesses a non-halogen counterion. In other embodiments the ammonium silane in the pharmaceutical composition possesses a halogen counterion. In other embodiments the ammonium silane in the pharmaceutical composition is an internal salt (zwitterion).

### Embodiments of the Present Invention

In certain embodiments, all charges in compounds are balanced with either anions A⁻, A2⁻, or cations, e.g. the ammonium N atom, or M*.

### Embodiments of R¹

In certain embodiments, R¹ is:

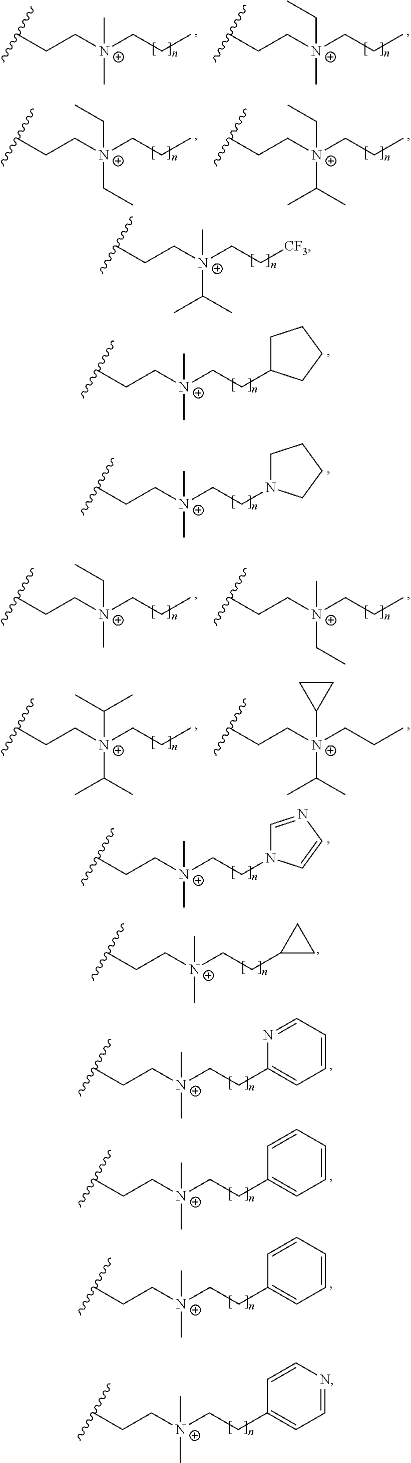

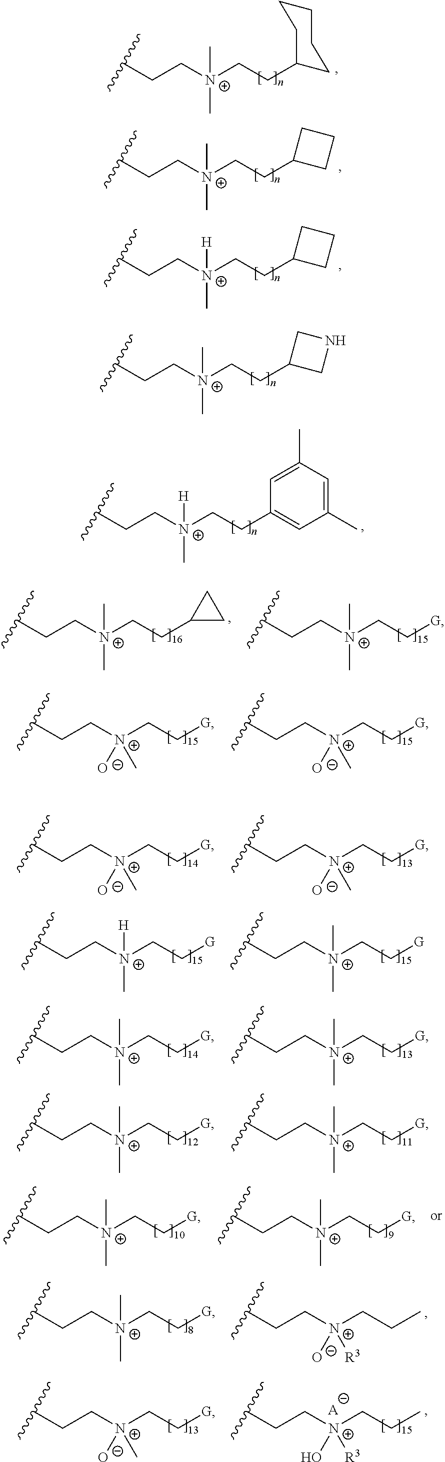

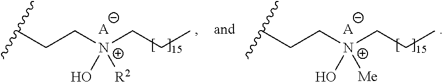

### Embodiments of R²

In certain embodiments, R² is hydrogen.

In certain embodiments, R² is methyl.

In certain embodiments, R² is ethyl.

In certain embodiments, R² is propyl.

In certain embodiments, R² is isopropyl.

In certain embodiments, R² is oxygen anion.

In certain embodiments, R² is hydroxyl.

### Embodiments of R³

In certain embodiments, R³ is hydrogen.

In certain embodiments, R³ is methyl.

In certain embodiments, R³ is ethyl.

In certain embodiments, R³ is propyl.

In certain embodiments, R³ is isopropyl.

In certain embodiments, R³ is oxygen anion.

### Embodiments of A⁻

In certain embodiments, A− is

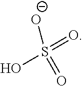

In certain embodiments, A− is

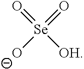

In certain embodiments, A− is

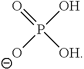

In certain embodiments, A− is

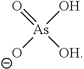

In certain embodiments, A− is

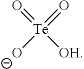

In certain embodiments, A− is

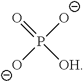

In certain embodiments, A− is

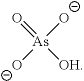

In certain embodiments, A− is

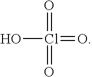

In certain embodiments, A− is

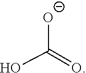

In certain embodiments, A− is

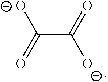

In certain embodiments, A− is

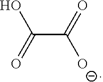

In certain embodiments, A− is

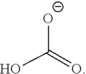

In certain embodiments, A− is

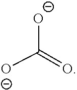

In certain embodiments, A− is

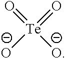

In certain embodiments, A− is

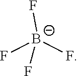

In certain embodiments, A− is

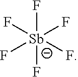

In certain embodiments, A− is

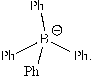 [SMILES: c1ccc([B-](c2ccccc2)(c2ccccc2)c2ccccc2)cc1]

In certain embodiments, A− is

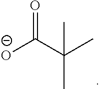

In certain embodiments, A− is

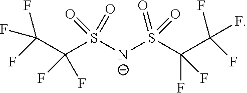 [SMILES: O=S(=O)([N-]S(=O)(=O)C(F)(F)C(F)(F)F)C(F)(F)C(F)(F)F]

In certain embodiments, A− is

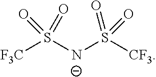 [SMILES: O=S(=O)([N-]S(=O)(=O)C(F)(F)F)C(F)(F)F]

In certain embodiments, A− is

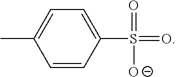

In certain embodiments, A− is

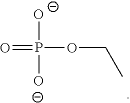

In certain embodiments, A− is

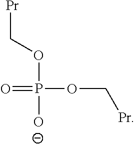

In certain embodiments, A− is

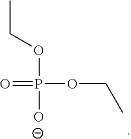

In certain embodiments, A− is

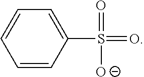

In certain embodiments, A− is

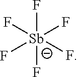

In a typically embodiment A− is not a halide.

In certain embodiments, A− is chloride.

In certain embodiments, A− is fluoride.

In certain embodiments, A− is bromide.

In certain embodiments, A− is iodide.

In certain embodiments, A− is chloride.

In certain embodiments, A− is nitrate.

In certain embodiments, A− is chlorite.

In certain embodiments, A− is perchlorate.

In certain embodiments, A− is hydroxide.

In certain embodiments, A− is formate.

In certain embodiments, A− is acetate.

In certain embodiments, A− is lactate.

In certain embodiments, A− is benzoate.

In certain embodiments, A− is nitrate.

In certain embodiments, A− is salicylate.

In certain embodiments, A− is

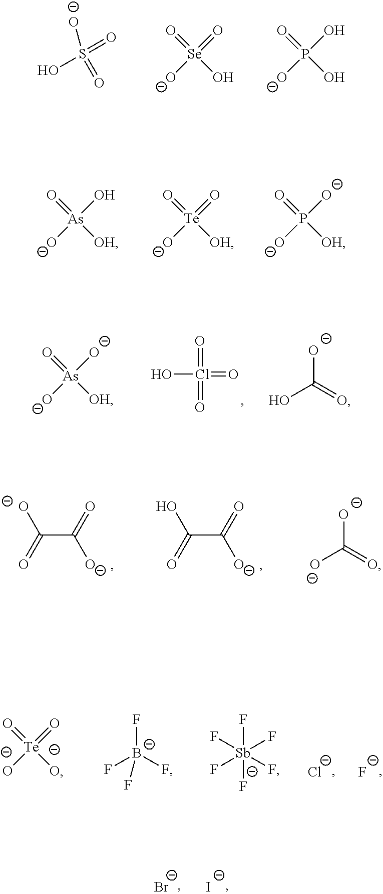

nitrate, chlorite, perchlorate, hydroxide, formate, acetate, lactate, benzoate, nitrate, or salicylate anion.

### Embodiments of A2⁻

In certain embodiments, A2 is

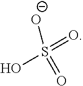

In certain embodiments, A2 is

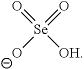

In certain embodiments, A2 is

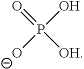

In certain embodiments, A2 is

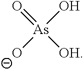

In certain embodiments, A2 is

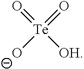

In certain embodiments, A2 is

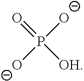

In certain embodiments, A2 is

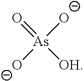

In certain embodiments, A2 is

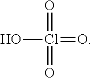

In certain embodiments, A2 is

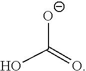

In certain embodiments, A2 is

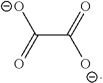

In certain embodiments, A2 is

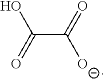

In certain embodiments, A2 is

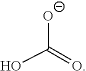

In certain embodiments, A2 is

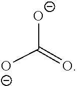

In certain embodiments, A2 is

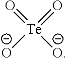

In certain embodiments, A2 is

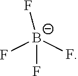

In certain embodiments, A2 is

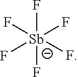

In certain embodiments, A2 is SCN—.

In certain embodiments, A2 is AcO—.

In certain embodiments, A2 is BzO—.

In certain embodiments, A2 is MeSO₃—.

In certain embodiments, A2 is Bu₂PO₄—.

In certain embodiments, A2 is nitrate.

In certain embodiments, A2 is chlorite.

In certain embodiments, A2 is perchlorate.

In certain embodiments, A2 is hydroxide.

In certain embodiments, A2 is formate.

In certain embodiments, A2 is acetate.

In certain embodiments, A2 is lactate.

In certain embodiments, A2 is benzoate.

In certain embodiments, A2 is nitrate.

In certain embodiments, A2 is salicylate.

In certain embodiments, A2⁻ is

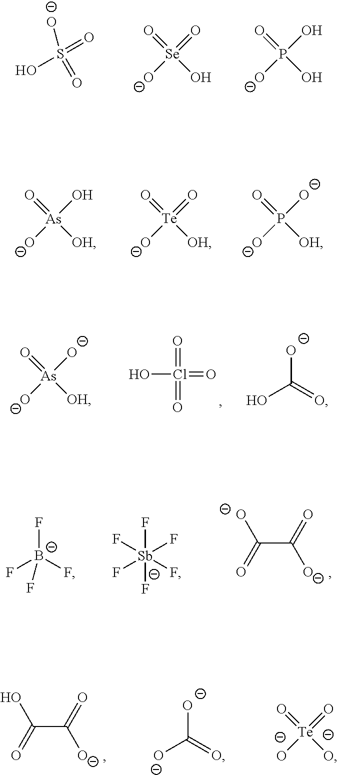

chlorite, perchlorate, hydroxide, formate, acetate, lactate, benzoate, nitrate, or salicylate anion.

### Embodiments of Formula A

In certain embodiments, the compound of Formula A is

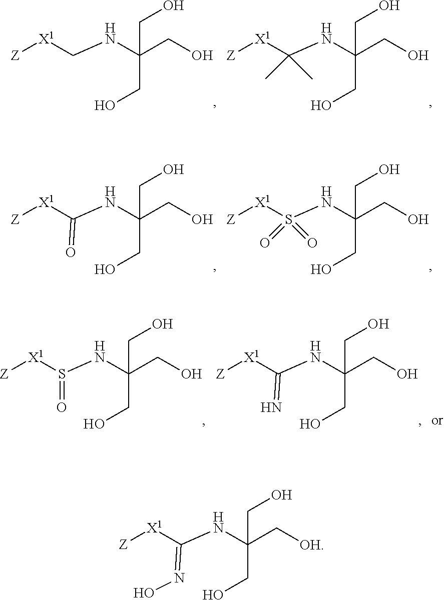

In certain embodiments, the compound of Formula A is selected from:

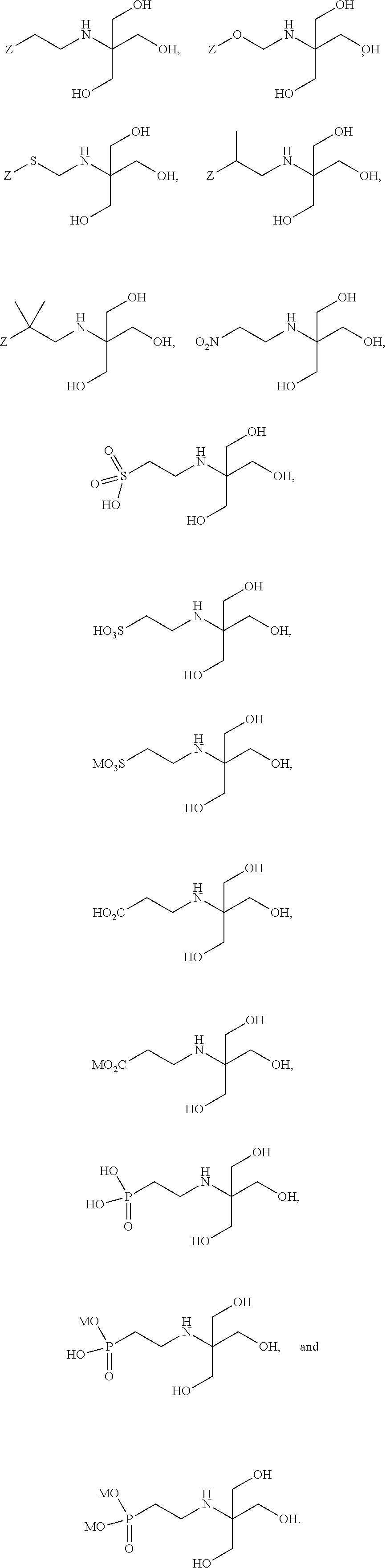

In certain embodiments, Formula A is selected from:

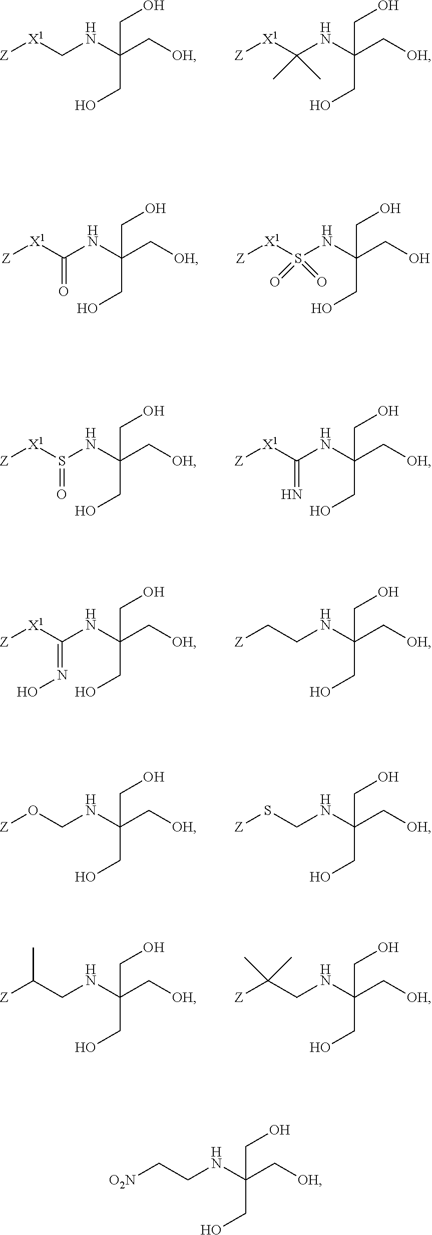

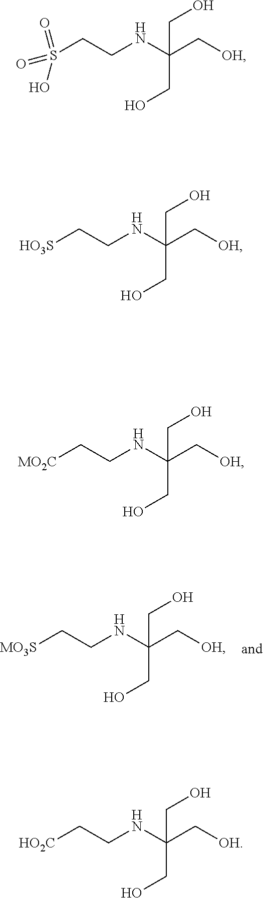

In certain embodiments, Formula A is selected from:

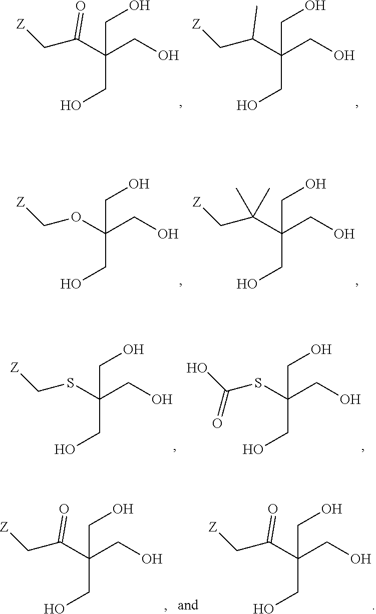

In certain embodiments, Formula A is selected from:

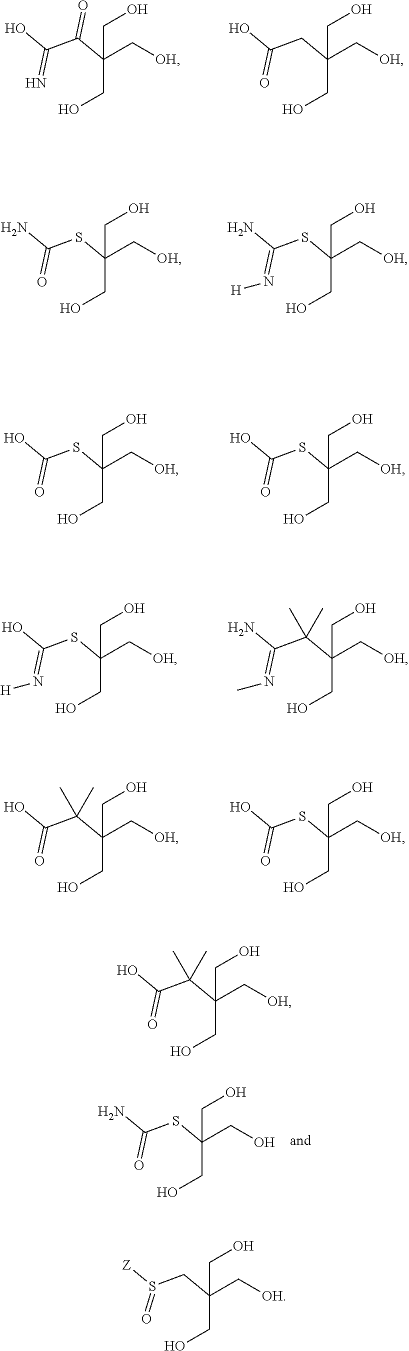

In certain embodiments, Formula A is selected from:

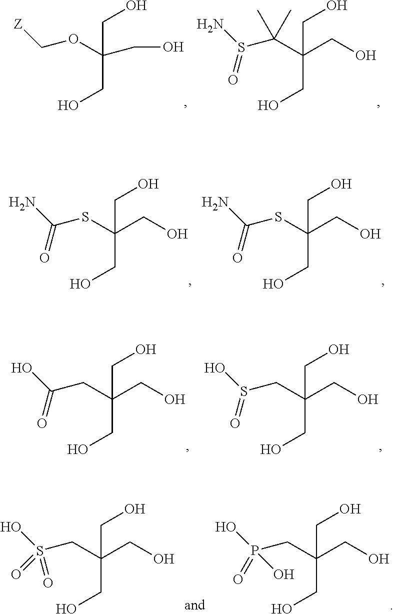

### Embodiments of R^{X}

In certain embodiments, R^{X} is selected from:

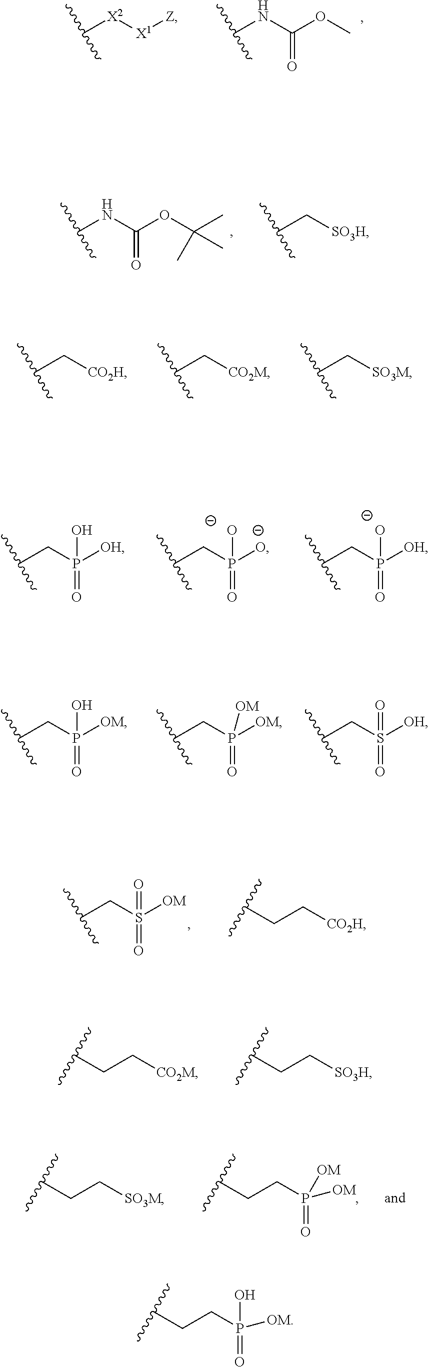

### Embodiments of X¹

In certain embodiments, X¹ is

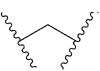 [SMILES: *C*]

In certain embodiments, X¹ is

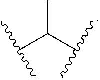 [SMILES: *C(*)C]

In certain embodiments, X¹ is

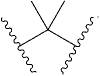 [SMILES: *C(*)(C)C]

In certain embodiments, X¹ is

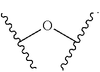 [SMILES: *O*]

In certain embodiments, X¹ is

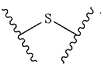 [SMILES: *S*]

### Embodiments of X²

In certain embodiments, X² is

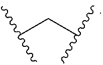 [SMILES: *C*]

In certain embodiments, X² is

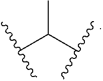 [SMILES: *C(*)C]

In certain embodiments, X² is

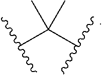 [SMILES: *C(*)(C)C]

In certain embodiments, X² is

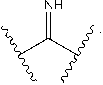 [SMILES: *C(*)=N]

In certain embodiments, X² is

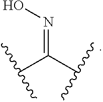 [SMILES: *C(*)=NO]

In certain embodiments, X² is

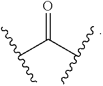 [SMILES: *C(*)=O]

In certain embodiments, X² is

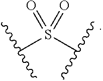 [SMILES: *S(*)(=O)=O]

In certain embodiments, X² is

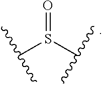 [SMILES: *S(*)=O]

### Embodiments of Z

In certain embodiments, Z is —NO₂.

In certain embodiments, Z is —SO₃H.

In certain embodiments, Z is —SO₂H.

In certain embodiments, Z is —SO₃⁻.

In certain embodiments, Z is —SO₃M.

In certain embodiments, Z is CO₂H.

In certain embodiments, Z is phosphate.

In certain embodiments, Z is OH.

In certain embodiments, Z is NH₂.

In certain embodiments, Z is

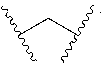 [SMILES: *C*]

In certain embodiments, Z is alkyl.

In certain embodiments, Z is C₁-C₆ alkyl.

### Synthesis

In some embodiments, the synthesis of an ammonium silane described herein is represented by the following scheme:

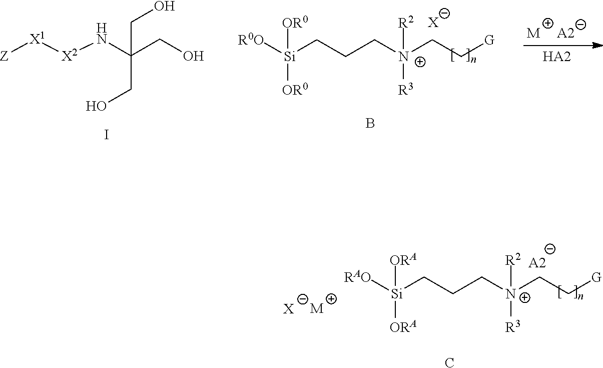

In this fashion, the alkoxide substituents from a compound of Formula B are replaced with one, two, or three formula I substituents bonded to the silicon atom via the oxygen. For example, in the case of a single substitution, the synthesis can be represented schematically as follows:

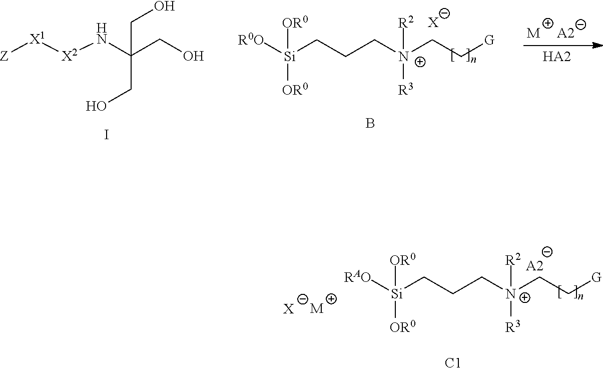

In other embodiments, a bis-substituted compound with two alkoxide substituents from the compound of Formula B is replaced with the Formula I substituent. For example,

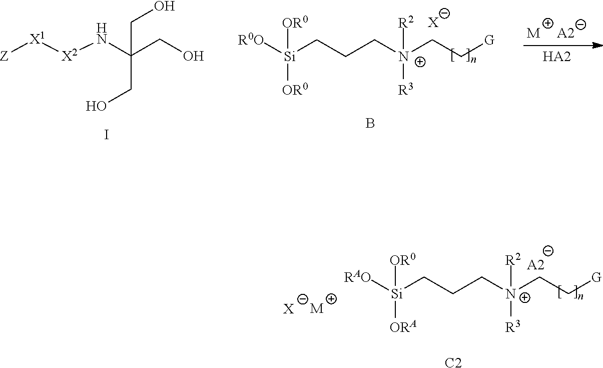

In a similar fashion, it is understood that in addition to compounds of Formula C with full substitution, active compounds covered by the invention include mono-and bis-substituted compounds of Formulas C1 and C2. Control of the reaction conditions on a case-by-case basis controls the types of products formed. In certain embodiments, about one equivalent of I is used. In certain embodiments about two equivalents of I are used. In certain embodiments about three equivalents of I are used.

In some embodiments, the synthesis of an ammonium silane described herein is represented by the following scheme:

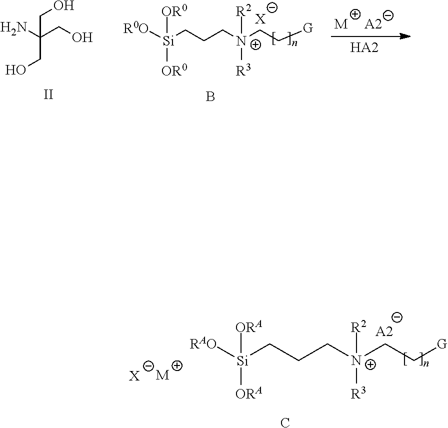

The alkoxide substituents from a compound of Formula B are replaced with one, two, or three formula II substituents bonded to the silicon atom via the oxygen. For example, in the case of a single substitution, the synthesis can be represented schematically as follows:

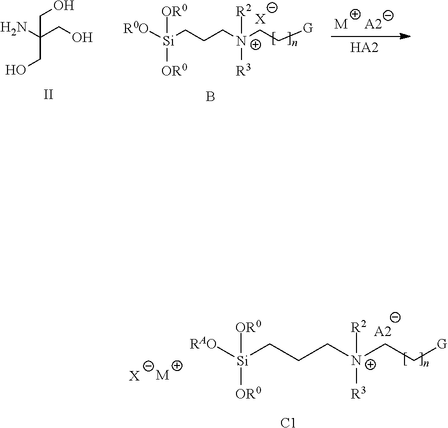

In the instance of a bis-substitution, the synthesis of an ammonium silane described herein is represented by the following scheme:

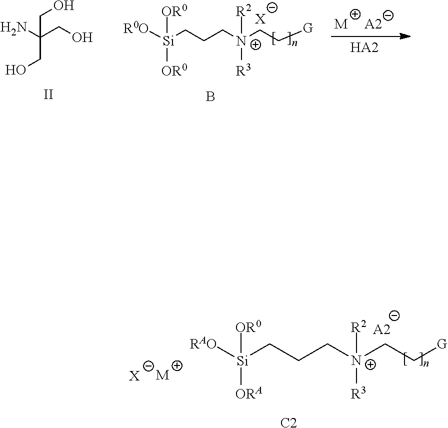

In certain embodiments, about one equivalent of II is used. In certain embodiments about two equivalents of II are used. In certain embodiments about three equivalents of II are used.

In some embodiments, the synthesis of an ammonium silane described herein is represented by the following scheme:

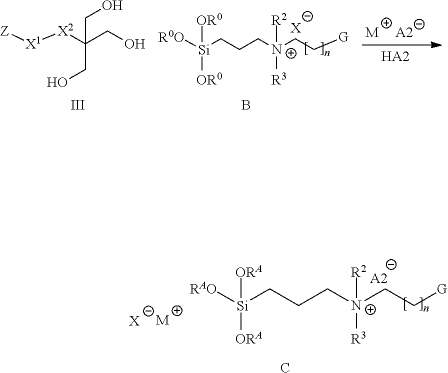

In addition to compounds of formula C, the instant invention includes compounds of formula C1:

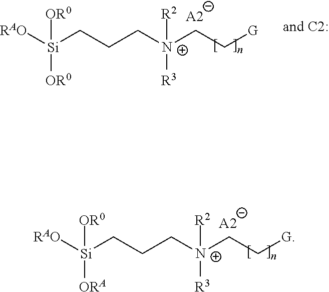

Because these compounds are formed during the formation of compounds of Formula C, they are part of the instant invention.

In certain embodiments, about one equivalent of III is used. In certain embodiments about two equivalents of III are used. In certain embodiments about three equivalents of III are used. The exact nature of the products formed depends on the molar ratios of reactants and reagents, temperature, concentration, and other factors.

In some embodiments, the synthesis of an ammonium silane described herein is represented by the following scheme:

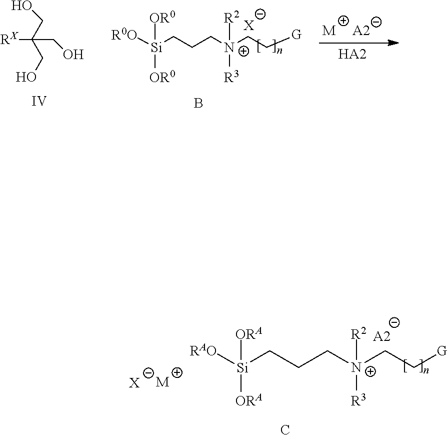

In addition to compounds of formula C, the instant invention enables the isolation of compounds of formula C1:

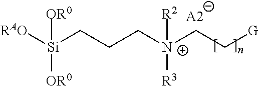

### C2:

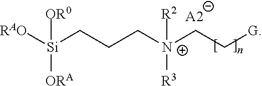

In certain embodiments, about one equivalent of IV is used. In certain embodiments about two equivalents of IV are used. In certain embodiments about three equivalents of IV are used.

In other embodiments, the syntheses of an ammonium silane described herein is given by the following scheme: A Compound of formula HI, HII, HIII, HIV, HV, HVI, HVII, HVIII, HIX, or HX is reacted with a compound of formula B to give a compound of formula Hc.

Compound of Formulas HI-HX B

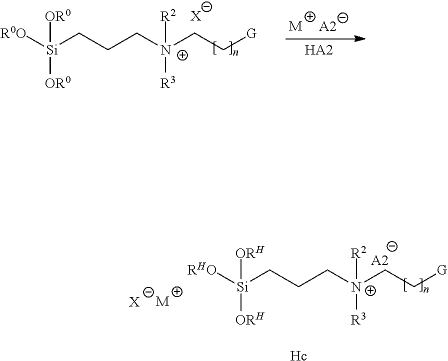

In other embodiments, the syntheses of an ammonium silane described herein is given by the following scheme: A Compound of formula HI, HII, HIII, HIV, HV, HVI, HVII, HVIII, HIX, or HX is reacted with a compound of formula B to give a compound of formula Hc.

Compound of Formulas HI-HXII B

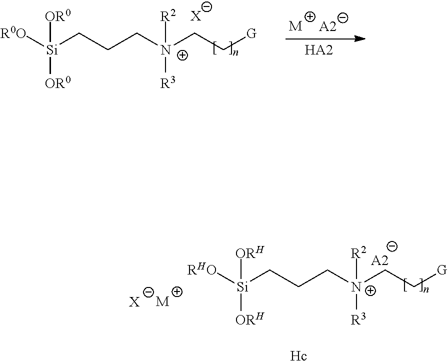

In addition to compounds of formula C, the instant invention enables the isolation of compounds of formula Hc1:

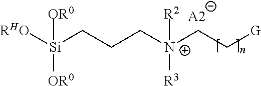

### and Hc2:

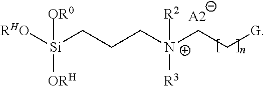

In these embodiments, the compound of formulas HI-HXI is selected from formulas HI-HX below:

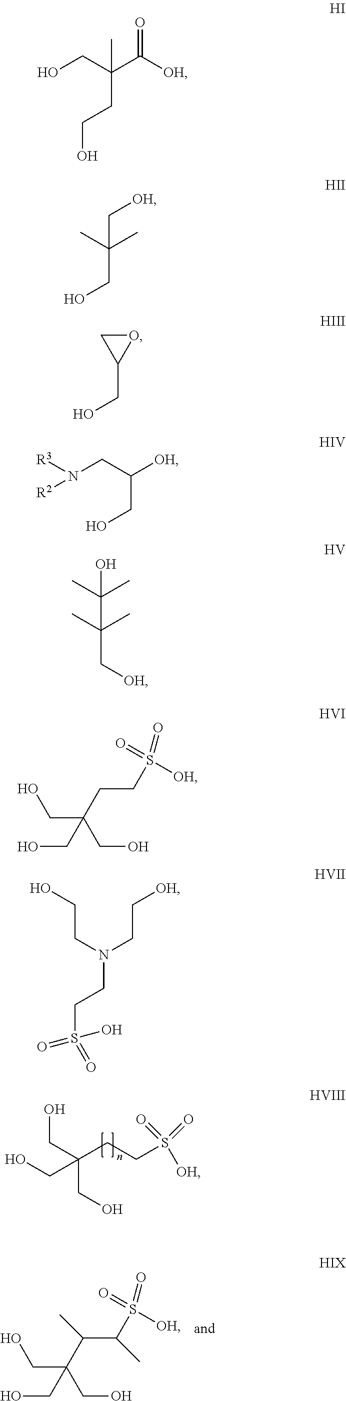

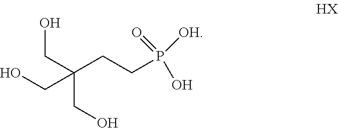 [SMILES: O=P(O)(O)CCC(CO)(CO)CO]

In certain embodiments, about one equivalent of HI, HII, HIII, HIV, HV, HVI, HVII, HVIII, HIX, or HX is used. In certain embodiments about two equivalents of HI, HII, HIII, HIV, HV, HVI, HVII, HVIII, HIX, or HX are used. In certain embodiments about three equivalents of HI, HII, HIII, HIV, HV, HVI, HVII, HVIII, HIX, or HX are used.

In some embodiments, the synthesis of an ammonium silane described herein is represented by the following scheme:

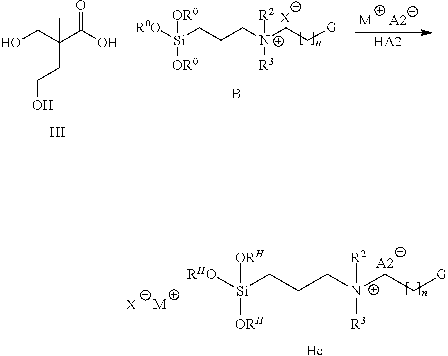

In certain embodiments, about one equivalent of HI is used. In certain embodiments about two equivalents of HI are used. In certain embodiments about three equivalents of HI are used.

In some embodiments, the synthesis of an ammonium silane described herein is represented by the following scheme:

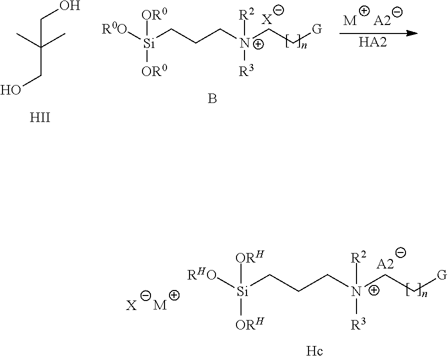

In certain embodiments, about one equivalent of HII is used. In certain embodiments about two equivalents of HII are used. In certain embodiments about three equivalents of HII are used.

In some embodiments, the synthesis of an ammonium silane described herein is represented by the following scheme:

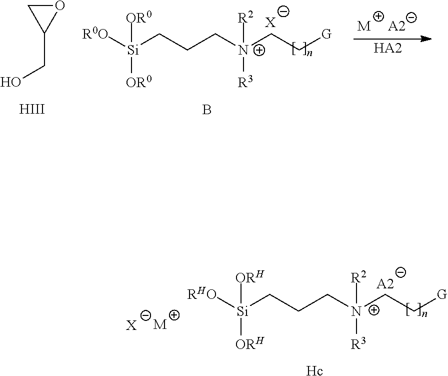

In certain embodiments, about one equivalent of HIII is used. In certain embodiments about two equivalents of HIII are used. In certain embodiments about three equivalents of HIII are used.

In some embodiments, the synthesis of an ammonium silane described herein is represented by the following scheme:

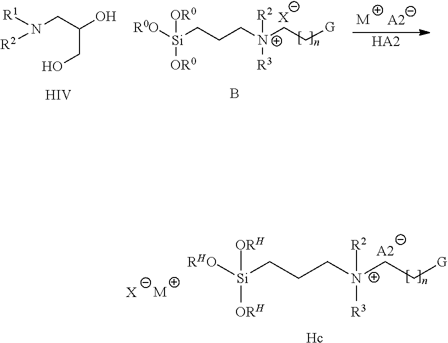

In certain embodiments, about one equivalent of HIV is used. In certain embodiments about two equivalents of HIV are used. In certain embodiments about three equivalents of HIV are used.

In some embodiments, the synthesis of an ammonium silane described herein is represented by the following scheme:

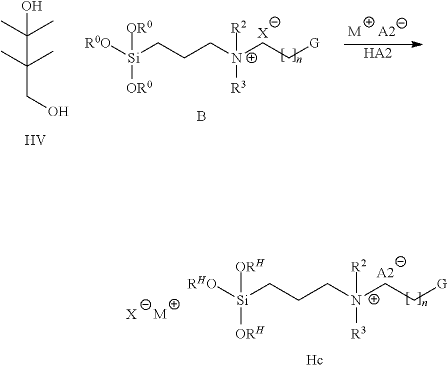

In certain embodiments, about one equivalent of HV is used. In certain embodiments about two equivalents of HV are used. In certain embodiments about three equivalents of HV are used.

In some embodiments, the synthesis of an ammonium silane described herein is represented by the following scheme:

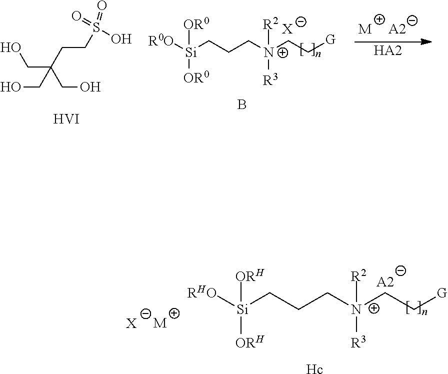

In certain embodiments, about one equivalent of HVI is used. In certain embodiments about two equivalents of HVI are used. In certain embodiments about three equivalents of HVI are used.

In some embodiments, the synthesis of an ammonium silane described herein is represented by the following scheme:

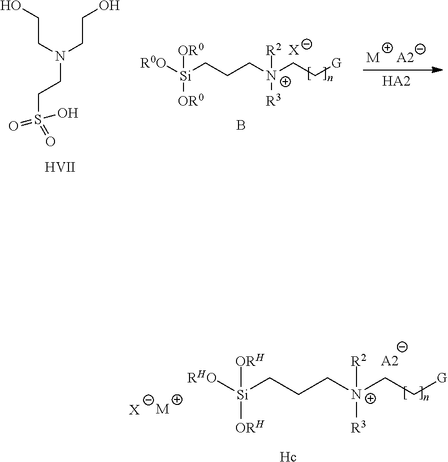

In certain embodiments, about one equivalent of HVII is used. In certain embodiments about two equivalents of HVII are used. In certain embodiments about three equivalents of HVII are used.

In some embodiments, the synthesis of an ammonium silane described herein is represented by the following scheme:

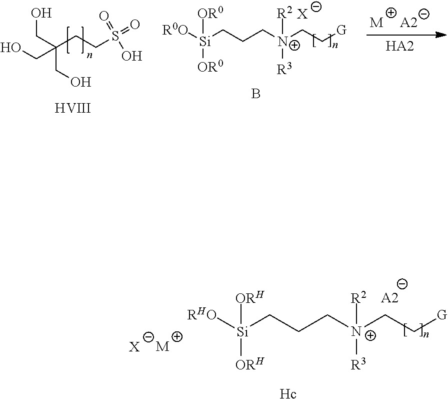

In certain embodiments, about one equivalent of HVIII is used. In certain embodiments about two equivalents of HVIII are used. In certain embodiments about three equivalents of HVIII are used.

In some embodiments, the synthesis of an ammonium silane described herein is represented by the following scheme:

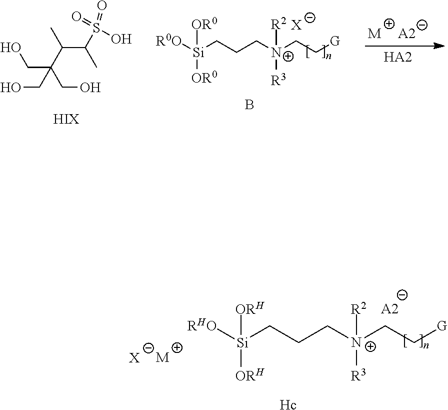

In certain embodiments, about one equivalent of HIX is used. In certain embodiments about two equivalents of HIX are used. In certain embodiments, about three equivalents of HIX are used.

In some embodiments, the synthesis of an ammonium silane described herein is represented by the following scheme:

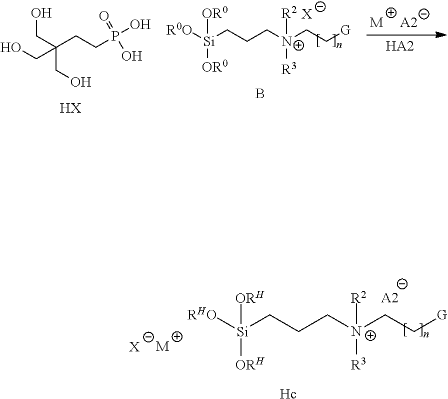

In certain embodiments, about one equivalent of HX is used. In certain embodiments, about two equivalents of HX are used. In certain embodiments, about three equivalents of HX are used.

In some embodiments, the synthesis of an ammonium silane described herein is represented by the following scheme:

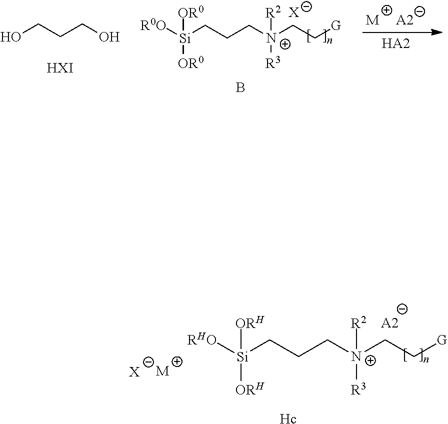

In certain embodiments, about one equivalent of HXI is used. In certain embodiments, about two equivalents of HXI are used. In certain embodiments, about three equivalents of HXI are used.

In some embodiments, the synthesis of an ammonium silane described herein is represented by the following scheme:

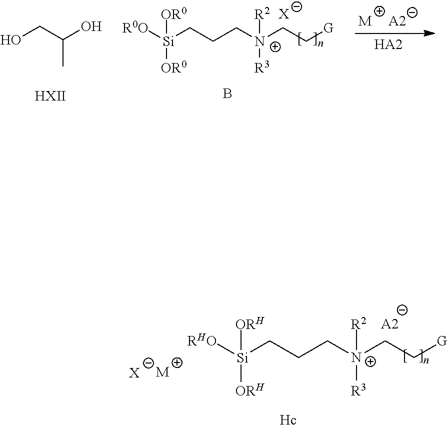

In certain embodiments, about one equivalent of HXII is used. In certain embodiments, about two equivalents of HXII are used. In certain embodiments, about three equivalents of HXII are used.

The molar ratio of a compound of formulas HI-HX or HI-HXII to a compound of formula B in the synthesis of the ammonium silane of type He will dictate the nature of the resulting pharmaceutical composition. In some embodiments, the ratio of a compound of Formulas HI-HX to a compound of Formula B can be 1:1, 1.10:1.0, 1.2:1, 1.3:1, 1.4:1, 1.51, 1.6:1, 1.7:1, 1.8:1, 1.9:1, 2:1, 1.10:1.0, 2.2:1, 2.3:1, 2.4:1, 2.51, 2.6:1, 2.7:1, 2.8:1, 2.9:1, or 3:1.

A compound of formulas HI, HII, HIII, HIV, HV, HVI, HVII, HVIII, HIX, HX, HXI, or HXII is reacted with a compound of type B in specific molar ratios, to generate ammonium silanes of type Hc. The relative concentration of each species in the pharmaceutical composition described herein are dictated by acid-base and exchange at silicon thermodynamic equilibrium principles.

In some embodiments, the synthesis of an ammonium silane described herein is represented by the following scheme:

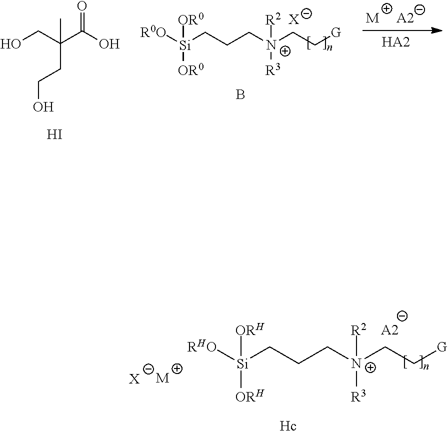

In some embodiments, the synthesis of an ammonium silane described herein is represented by the following scheme:

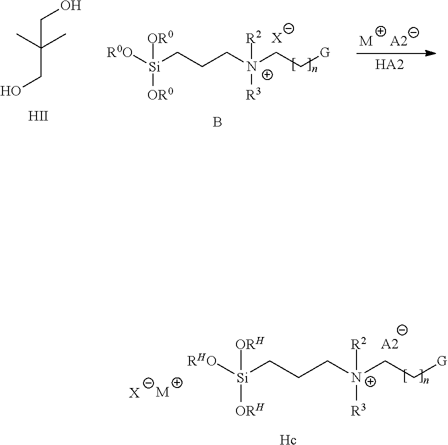

In some embodiments, the synthesis of an ammonium silane described herein is represented by the following scheme:

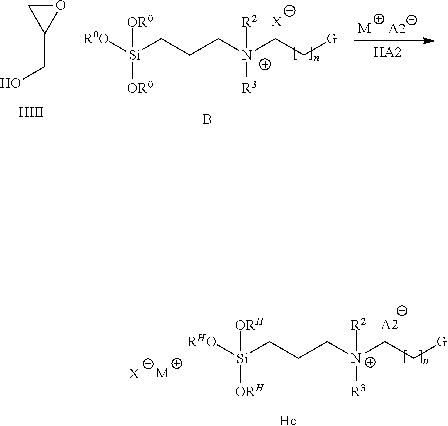

In some embodiments, the synthesis of an ammonium silane described herein is represented by the following scheme:

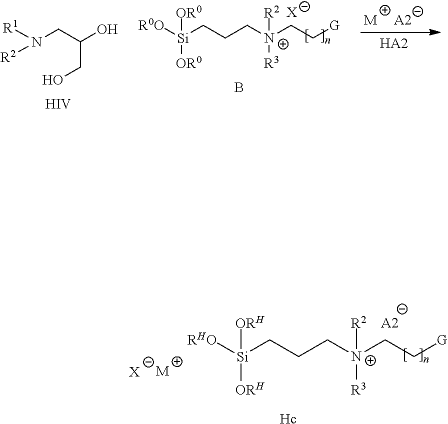

In some embodiments, the synthesis of an ammonium silane described herein is represented by the following scheme:

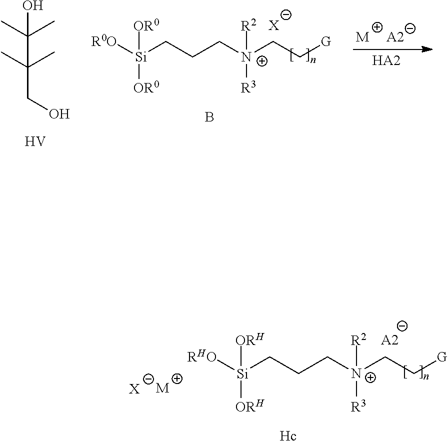

In some embodiments, the synthesis of an ammonium silane described herein is represented by the following scheme:

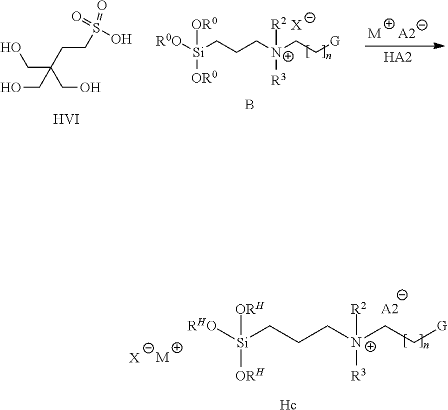

In some embodiments, the synthesis of an ammonium silane described herein is represented by the following scheme:

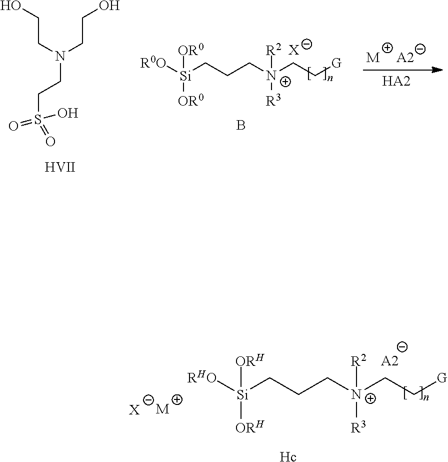

In some embodiments, the synthesis of an ammonium silane described herein is represented by the following scheme:

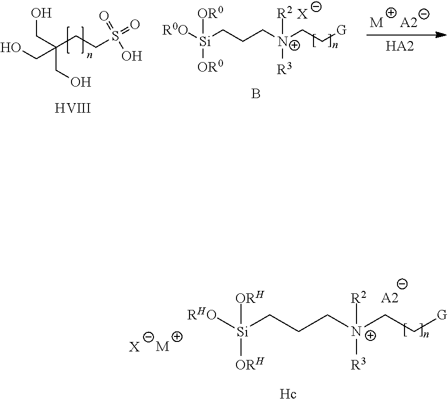

In some embodiments, the synthesis of an ammonium silane described herein is represented by the following scheme:

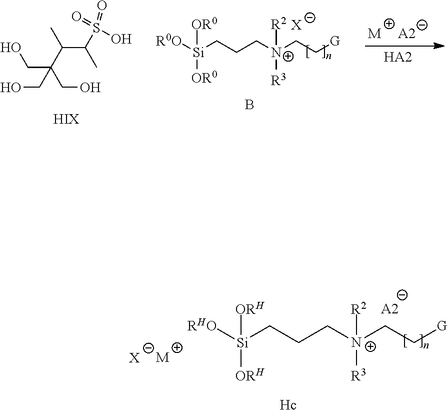

In some embodiments, the synthesis of an ammonium silane described herein is represented by the following scheme:

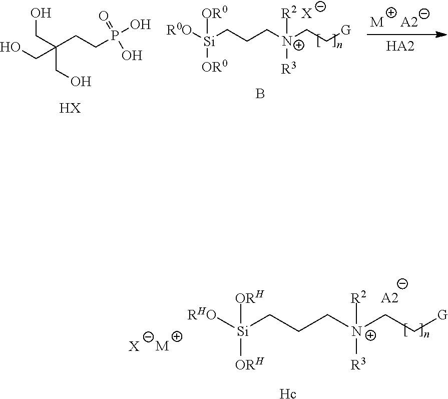

The molar ratio of a compound of formulas HI-HX or I-IV to a compound of formula B in the synthesis of the ammonium silanes of type C or He will dictate the nature of the resulting pharmaceutical composition. In some embodiments, the ratio of a compound of Formulas HI-HX or I-V to a compound of Formula B can be 1.0:1.0, 1.10:1.0, 1.2:1, 1.3:1, 1.4:1, 1.51, 1.6:1, 1.7:1, 1.8:1, 1.9:1, 2:1, 1.10:1.0, 2.2:1, 2.3:1, 2.4:1, 2.51, 2.6:1, 2.7:1, 2.8:1, 2.9:1, or 3:1.

It is known that under certain conditions, the ammonium silanes described herein can react with water to form mixtures of hydroxy/alkoxy silanes. Depending on the pH and the relative amount of buffer added to the mixture, the rate of hydrolysis may be controlled.

When used topically, the pharmaceutical compositions described here may exist in a variety of forms depending on the pH, type of the ammonium silane, and other factors. As such, the invention comprises each of these possible intermediates and their cumulative pharmaceutical effect.

### Definitions

Compounds are described using standard nomenclature. Unless defined otherwise, all technical and scientific terms used herein have the same meaning as is commonly understood by one of skill in the art to which this invention belongs.

The ammonium silanes in any of the Formulas described herein include racemates, enantiomers, mixtures of enantiomers, diastereomers, mixtures of diastereomers, tautomers, isomers; such as rotamers, as if each is specifically described.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The present invention includes ammonium silanes with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into ammonium silanes of the invention include isotopes of hydrogen, carbon, nitrogen, oxygen, fluorine, chlorine and iodine such as ²H, ³H, ¹¹C, ¹³C, ¹⁴C, ¹⁵N, ¹⁷O, ¹⁸O ¹⁸F, ³⁶Cl, and ¹²⁵I respectively. In one non-limiting embodiment, isotopically labelled ammonium silanes can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled ammonium silanes of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is at least about 90, 95 or 99% or more enriched in an isotope at any location of interest. In one non-limiting embodiment, deuterium is at least about 90, 95 or 99% enriched at a desired location.

In one non-limiting embodiment, the substitution of one or more hydrogen atoms for a deuterium atoms can be provided in any of the compounds described herein. In one non-limiting embodiment, the substitution of a hydrogen atom for a deuterium atom occurs within a group described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.).

In certain embodiments an ammonium silane described herein may form a solvate with one or more solvents (for example water). Therefore, in one non-limiting embodiment, the invention includes a solvated form of the ammonium silane. The term “solvate” refers to a molecular complex of a compound described herein (including a salt thereof) with one or more solvent molecules. Non-limiting examples of solvents are water, ethanol, dimethyl sulfoxide, acetone and other common organic solvents. The term “hydrate” refers to a molecular complex comprising an ammonium silane of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

A dash (“—”) that is not between two letters or symbols is used to indicate a point of attachment for a substituent. For example, —(C═O)NH₂ is attached through carbon of the keto (C═O) group.

The term “Group I” herein refers to Group I alkali metals, specifically lithium (Li), sodium (Na), potassium (K), rubidium (Rb), and caesium (Cs). “Group II” herein refers to Group II alkali earth metals, specifically beryllium (Be), magnesium (Mg), calcium (Ca), strontium (Sr), and barium (Ba). “Group III” herein refers to aluminum. “Transition metals” herein refers to any element in the d-block, f-block lanthanide and actinide of the periodic table. Also included are post-transition metals, specifically, gallium, indium, tin, thallium, and lead.

“Aryl” indicates aromatic groups containing only carbon in the aromatic ring or rings. In some embodiments, the aryl groups contain 1 to 3 separate or fused rings and is 6 to about 14 or 18 ring atoms, without heteroatoms as ring members. As used in this application, the term “aryl” refers to an unsubstituted aryl group unless indicated otherwise. For example, C₆ aryl is phenyl; and C₆ aryl which is optionally substituted with 1, 2, or 3 substituents independently selected from halogen, alkyl, and haloalkyl, includes the following non-limiting examples:

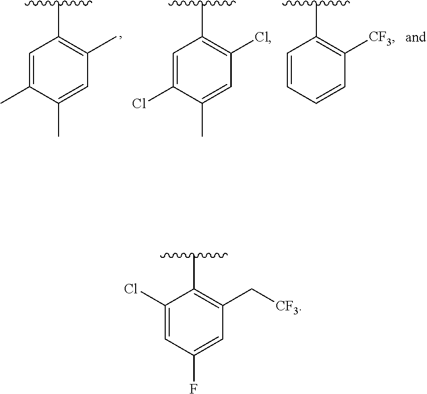

When indicated, such aryl groups may be further substituted with carbon or non-carbon atoms or groups. Such substitution may include fusion to a 3 to 7-membered saturated cyclic group that optionally contains 1 or 2 heteroatoms independently chosen from N, O, and S, to form, for example, a 3,4-methylenedioxyphenyl group. Aryl groups include, for example, phenyl and naphthyl, including 1-naphthyl and 2-naphthyl. In some embodiments, aryl groups are pendant.

An example of a pendant ring is a phenyl group substituted with a phenyl group. In some embodiments, the aryl group is optionally substituted as described above. “Alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. As used in this application, the term “alkyl” refers to an unsubstituted alkyl group unless indicated otherwise. For example, C₃ alkyl is propyl; and C₃ alkyl which is optionally substituted with 1, 2, or 3 substituents independently selected from halogen, alkyl, and haloalkyl, includes the following non-limiting examples:

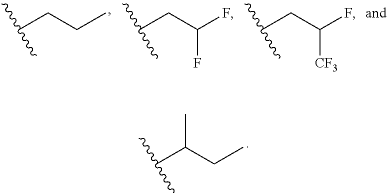

In an alternative embodiment, the alkyl group is optionally substituted. The term “Alkyl” also encompasses cycloalkyl or carbocyclic groups. For example, when a term is used that includes “alk” then “cycloalkyl” or “carbocyclic” can be considered part of the definition, unless unambiguously excluded by the context. For example, and without limitation, the terms alkyl, haloalkyl, etc. can all be considered to include the cyclic forms of alkyl, unless unambiguously excluded by context.

“Halo” and “Halogen” is fluorine, chlorine, bromine, or iodine.

“Haloalkyl” is a branched or straight-chain alkyl groups substituted with 1 or more halogen atoms, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include but are not limited to, trifluoromethyl and pentafluoroethyl.

The term “heterocyclyl,” “heterocycle,” or “heterocyclic ring” as used herein refers to a saturated or a partially unsaturated (i.e., having one or more double and/or triple bonds within the ring without aromaticity) carbocyclic radical of 3 to about 12, and more typically 3, 5, 6, 7 to 10 ring atoms in which at least one ring atom is a heteroatom selected from nitrogen, oxygen, phosphorus and sulfur, the remaining ring atoms being C, where one or more ring atoms is optionally substituted independently with one or more substituents described above. A heterocycle may be a monocycle having 3 to 7 ring members (2 to 6 carbon atoms and 1 to 4 heteroatoms selected from N, O, P, and S) or a bicycle having 6 to 10 ring members (4 to 9 carbon atoms and 1 to 6 heteroatoms selected from N, O, P, and S), for example: a bicyclo[4,5], [5,5], [5,6], or [6,6] system. In some embodiments, the only heteroatom is nitrogen. In some embodiments, the only heteroatom is oxygen. In some embodiments, the only heteroatom is sulfur. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Examples of heterocyclic rings include, but are not limited to, pyrrolidinyl, dihydrofuranyl, tetrahydrothienyl, tetrahydropyranyl, dihydropyranyl, tetrahydrothiopyranyl, piperidino, piperidonyl, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, dihydroisoquinolinyl, tetrahydroisoquinolinyl, pyrazolidinylimidazolinyl, imidazolidinyl, 2-oxa-5-azabicyclo[2.2.2]octane, 3-oxa-8-azabicyclo[3.2.1]octane, 8-oxa-3-azabicyclo[3.2.1]octane, 6-oxa-3-azabicyclo[3.1.1]heptane, 2-oxa-5-azabicyclo[2.2.1]heptane, 3-azabicyco[3.1.0]hexanyl, 3-azabicyclo[4.1.0]heptanyl, azabicyclo[2.2.2]hexanyl, 3H-indolyl, quinolizinyl, N-pyridyl ureas, and pyrrolopyrimidine. Spiro moieties are also included within the scope of this definition. Examples of a heterocyclic group wherein 1 or 2 ring carbon atoms are substituted with oxo (═O) moieties are pyrimidinonyl and 1,1-dioxo-thiomorpholinyl. As used in this application, the term “heterocyclyl” refers to an unsubstituted heterocyclyl group unless indicated otherwise. For example, heterocyclyl with 6 ring atoms with one of the ring atoms being nitrogen is piperidinyl; and piperidinyl which is optionally substituted with 1, 2, or 3 substituents independently selected from halogen, alkyl, and haloalkyl, includes the following non-limiting examples:

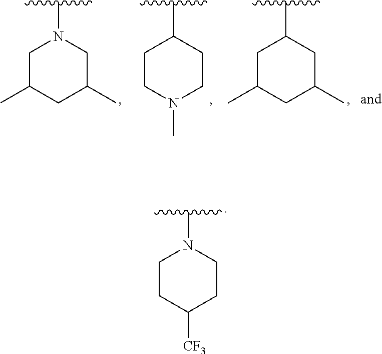

The heterocyclyl groups herein are optionally substituted independently with one or more substituents described herein.

The term “infection” as used herein refers both to microbial invasion with visual evidence of inflammatory response and also to biofilm infection in which obvious signs of clinical infection may be absent or replaced by exudation, non-healing, or inappropriate granulation, and in certain embodiments, for example, without a normal progression to epithelization.

The term “carrier” applied to compositions/combinations of the invention refers to a diluent, excipient, or vehicle with which an active compound is provided.

A “patient” or “host” or “subject” is a human or non-human animal in need of treatment or prevention of any of an infection as described herein. In some embodiments, the host is a human. A “patient” or “host” or “subject” also refers to for example, a mammal, primate (e.g., human), cows, sheep, goat, horse, dog, cat, rabbit, rat, mice, fish, bird and the like.

A “therapeutically effective amount” of a composition/combination of this invention means an amount effective, when administered to a host, to provide a therapeutic benefit such as an amelioration of symptoms or reduction or diminution of the disease itself. In some embodiments, a therapeutically effective amount is an amount sufficient to inhibit progression, cause a regression, cause a cure, or inhibit, eliminate, or prevent an infection in a host in need thereof.

As used herein, “salt” is a derivative of the disclosed compound in which the parent compound is modified by making inorganic or organic, non-toxic, acid or base addition salts thereof. In certain embodiments the compound is already a salt and the salt version adds one or more equivalents of salt (for example a TRIS containing compound wherein the basic nitrogen of TRIS can react with an acid to form a salt). The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reactive free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practical. Salts of the present compounds further include solvates of the compound and the compound salts.

Examples of salts include, but are not limited to, mineral or organic acid salts of basic residues such as amines; alkali or organic salts of acidic residues such as carboxylic acids; and the like. The salts include the conventional non-toxic salts and the ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like, or using a different acid that produces the same counterion. Lists of additional suitable salts may be found, e.g., in Remington's PharmaceuticalSciences, 17^{th} 20 ed., Mack Publishing Company, Easton, Pa., p. 1418 (1985).

The ammonium silanes described herein have increased stability in water in comparison to other commercially available salts, for example, 3-(trihydroxysilyl)-N-propyl-N,N-dimethyloctadecyl ammonium chloride. The inventors discovered that salts prepared with non-halide counterions showed increased water stability and decreased levels of polymerization to subsequently insoluble polysilsequioxane materials in both solid form and in solution. Additionally, formulations described herein are substantially free of alcohols such as methanol, ethanol and/or other unwanted small volatile organic compounds.

In certain embodiments the ammonium silanes described herein do not produce byproducts such as methanol upon hydrolysis. Powder or other solid formulations, for example lyophilized powder, described herein are substantially free of methanol, ethanol and/or other unwanted small volatile organic compounds due to either (i) not using such compounds in the manufacture or (ii) the use of vacuum drying during processing, a significant improvement over commercially available aqueous solutions of other organosilane antimicrobials, such as 3-(trihydroxysilyl)-N-propyl-N,N-dimethyloctadecyl ammonium chloride, which often contain significant quantities of methanol as a byproduct of its synthesis.

The compounds described herein are capable of prefusing a biofilm and can do so even as an oligomeric species; and are also stabile in aqueous or glycerin solution to traverse the small channels of a biofilm, providing increased penetration into the biofilm with efficacy to eliminate the entire biofilm, including persister cells.

Potency is retained after interacting with and killing microbes since the ammonium silane is not consumed by such interactions and can continue to be lethal to active microbes. Furthermore, the killing mechanism for these compounds diminishes the possibility of resistance development in targeted organisms compared to other topical infection treatments that use antibiotics and antifungals.

In some embodiments, an ammonium silane described herein is a solid compound that readily dissolves in water to form a stable aqueous solution. For example, the ammonium silane can be dissolved in saline solution, deionized water, or a buffer to form a visibly clear aqueous solution. In some embodiments, the aqueous solution can be stored at room temperature without clouding for at least 1, 2, 3, 4, 5, 6, 8, 12, 16, or more days. In another embodiment, the ammonium silane can be made more soluble by first applying a lyophilizer to make a powder.

In another embodiment, an ammonium silane described herein forms a solution that is or is nearly neutral when dissolved in water. For example, the aqueous solution can have a pH of approximately 6.4, 6.8, 7.0, 7.2, 7.4, 7.6, 7.8 or 8.0.

In some embodiments, the solutions described above have at least 0.2%, 0.4%, 0.6%, 0.8% 1%, 1.2%, 1.4%, 1.6%, 1.8%, or 2.0% concentration of an ammonium silane described herein.

In a typical embodiment, the positive charges in the formulas described herein are paired with one or more negatively charged ions. The inventors have discovered that a key factor in the stability of the trihydroxysilyl species of Formula B depends on the character of the counterion, as silyl compounds can undergo exchange reactions in water catalyzed by not only acidic conditions, but also by nucleophiles.

### Representative Compounds of the Present Invention

In certain embodiments, Formula A is

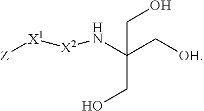

In certain embodiments, Formula A is

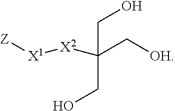

In certain embodiments, Formula A is

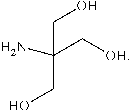

In certain embodiments, a compound of Formula A is selected from:
hydrogen,

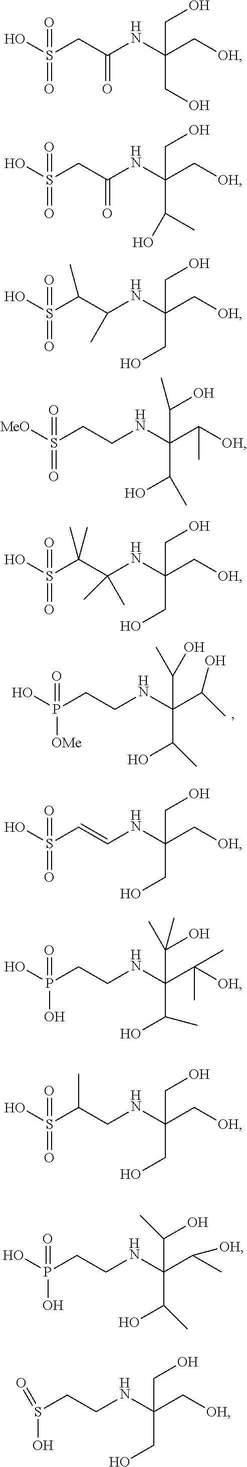

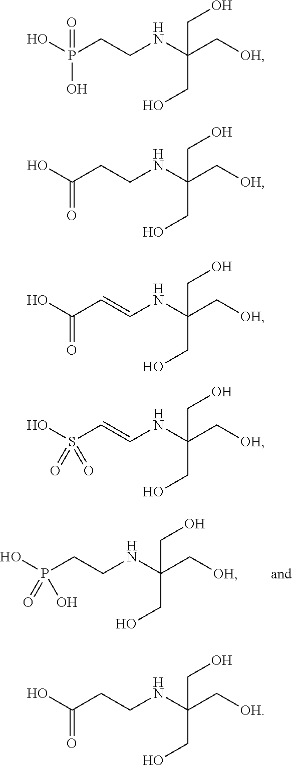

In certain embodiments, a compound of Formula H is selected from:

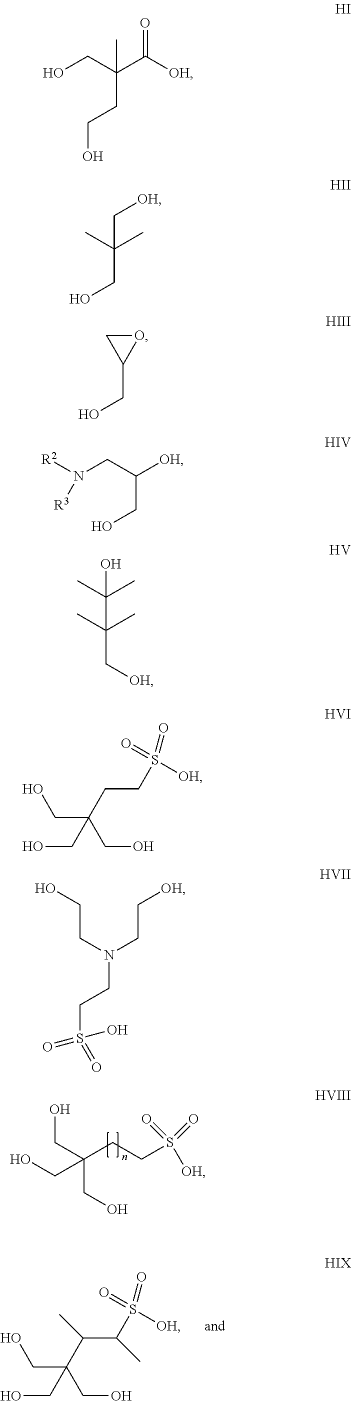

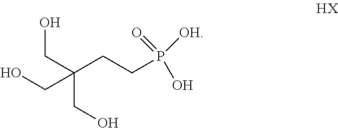 [SMILES: O=P(O)(O)CCC(CO)(CO)CO]

In certain embodiments, the compound of Formula A or Formulas H is bound to one silicon atom. Unless excluded by context the term Formula A may refer to compounds either as a substituent (R^{A}) or as the unbound Formulas (AI-AIV). Similarly, unless excluded by context the term Formula H may refer to compounds either as a substituent (R^{H}) or as unbound Formulas (HI-HX). For example, in certain embodiments, the R^{A} substituent can be selected from:

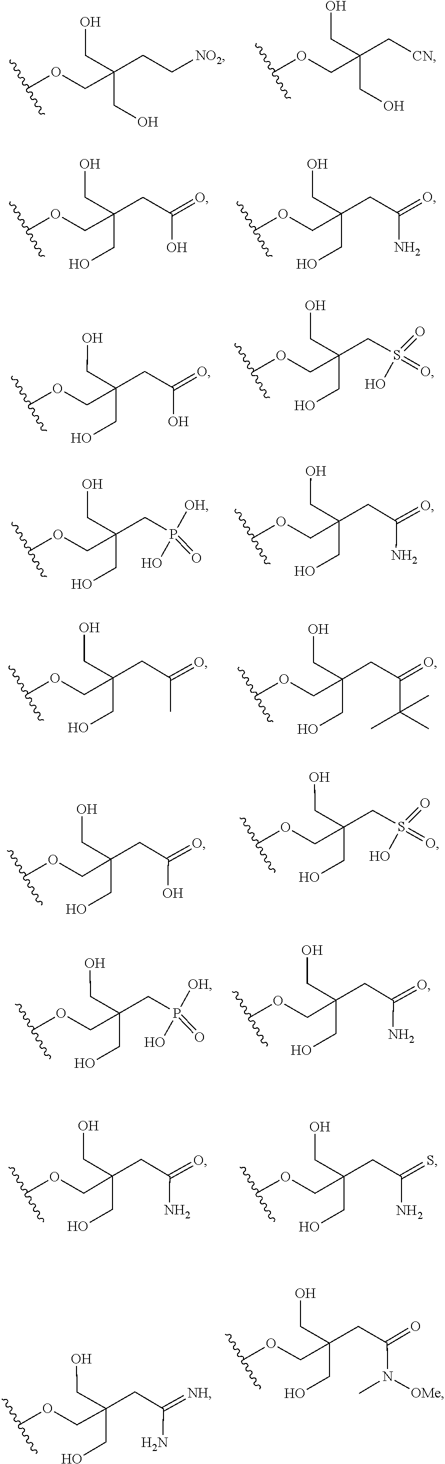

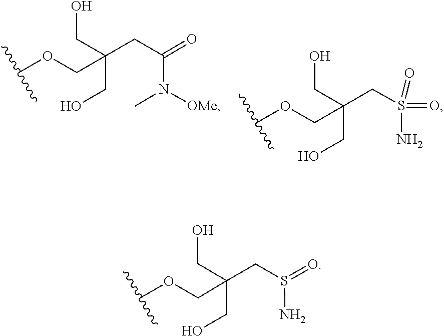 [SMILES: *OCC(CO)(CO)CC(=O)N(C)OC ; *OCC(CO)(CO)CS(N)(=O)=O ; *OCC(CO)(CO)CS(N)=O]

In certain embodiments, the R^{A} substituent is bound via a single bound to a single oxygen atom. In certain embodiments, the R^{A} substituent is selected from:

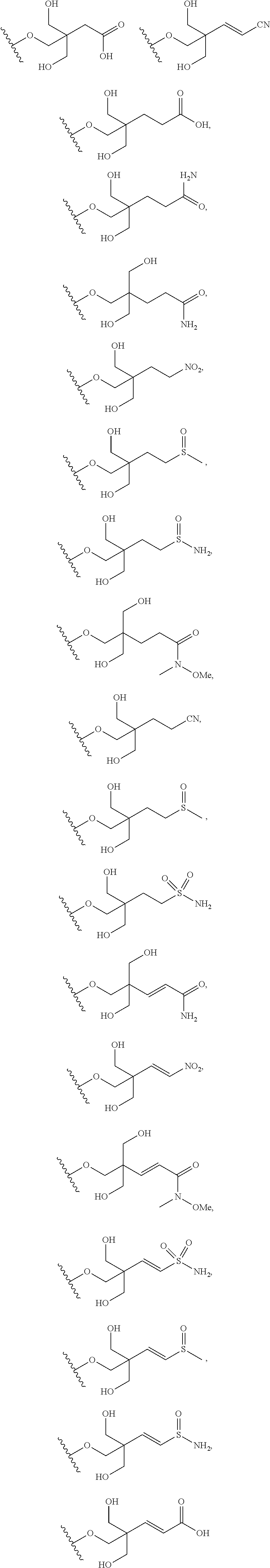

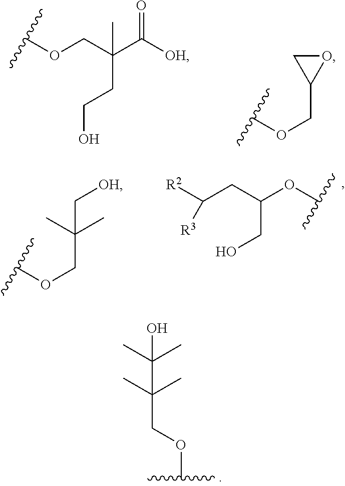

In certain embodiments, the R^{A} substituent is bound via a single bound to a single oxygen atom. In certain embodiments, the R^{A} substituent is selected from:

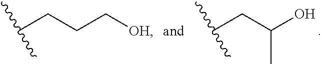

In certain embodiments, the R^{A} substituent invention has two single bonds to two different atoms. In certain embodiments, the R^{A} substituent is selected from:

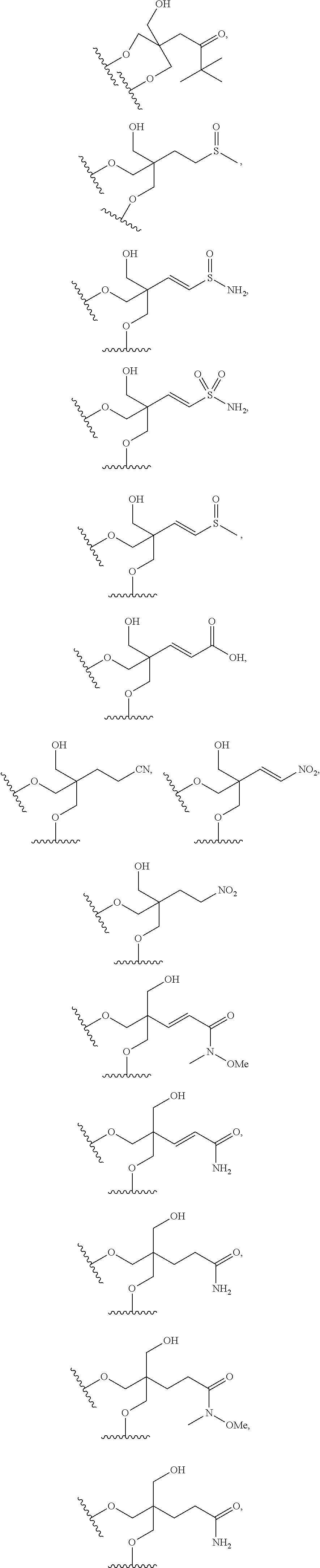

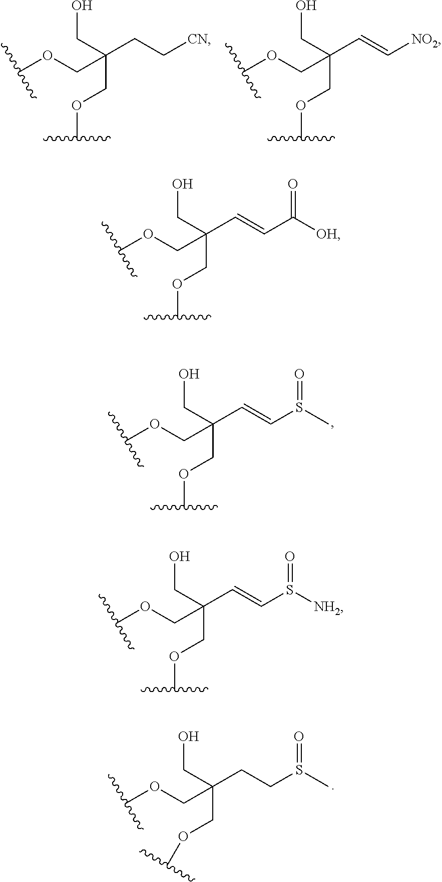

In certain embodiments, the R^{A} substituent has two single bonds to a single silicon atom. In certain embodiments, the R^{A} substituent is selected from:

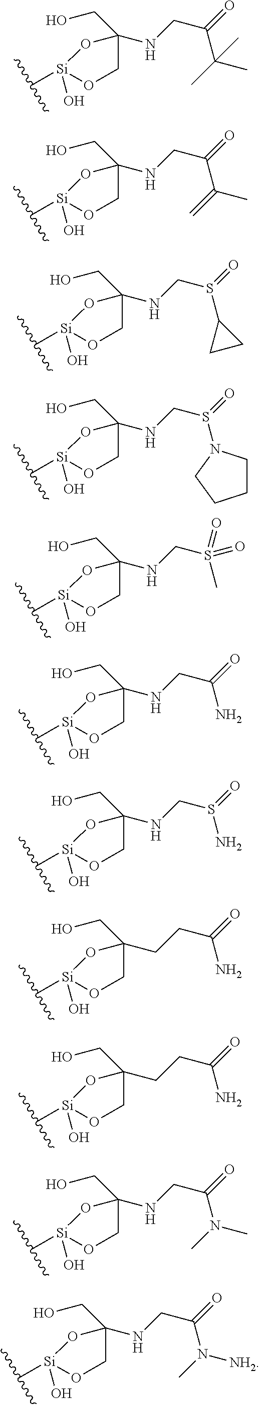

In certain embodiments, the R^{A} substituent has two single bonds to a single silicon atom. In certain embodiments, the R^{A} substituent is selected from:

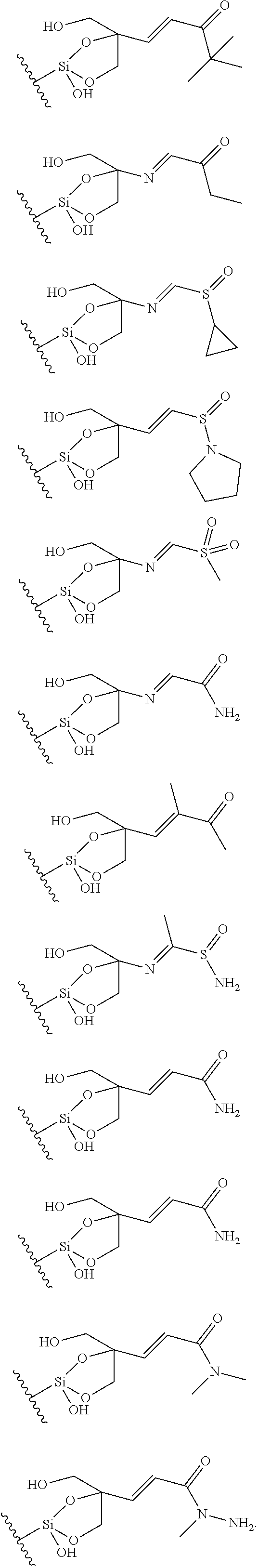

In certain embodiments, the R^{A} substituent joins the moiety via three single bonds to a single silicon atom. In certain embodiments, the R^{A} substituent is selected from:

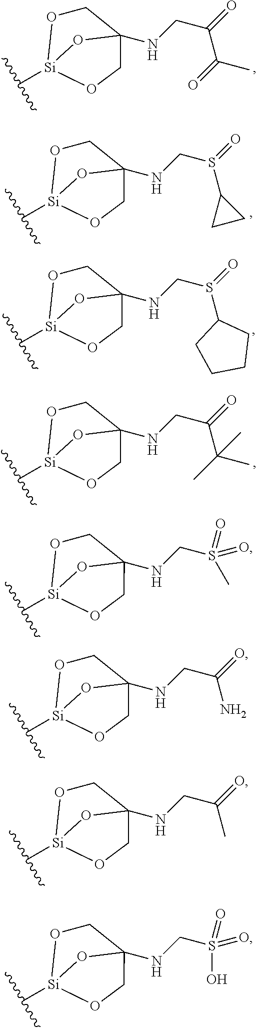

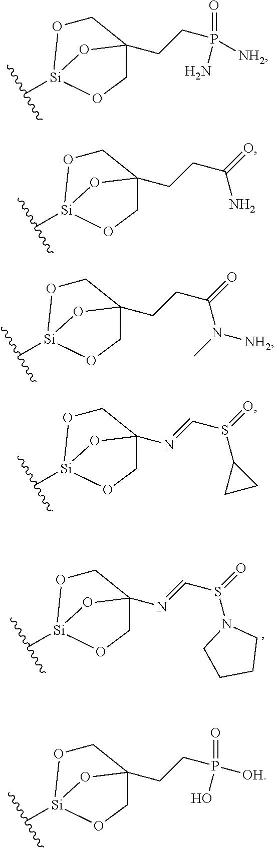 [SMILES: *[Si]12OCC(CCP(N)(N)=O)(CO1)O2 ; *[Si]12OCC(CCC(N)=O)(CO1)O2 ; *[Si]12OCC(CCC(=O)N(C)N)(CO1)O2 ; *[Si]12OCC(/N=C/S(=O)C3CC3)(CO1)O2 ; *[Si]12OCC(/N=C/S(=O)N3CCCC3)(CO1)O2 ; *[Si]12OCC(CCP(=O)(O)O)(CO1)O2]

In certain embodiments a compound is provided of structure

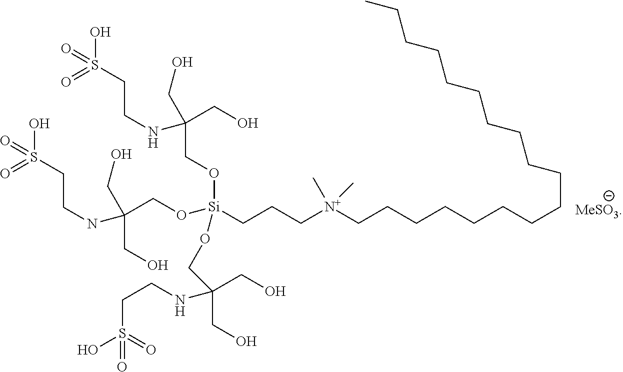 [SMILES: CCCCCCCCCCCCCCCCCC[N+](C)(C)CCC[Si](OCC(CO)(CO)NCCS(=O)(=O)O)(OCC(CO)(CO)NCCS(=O)(=O)O)OCC(CO)(CO)NCCS(=O)(=O)O]

In certain embodiments a compound is provided of structure

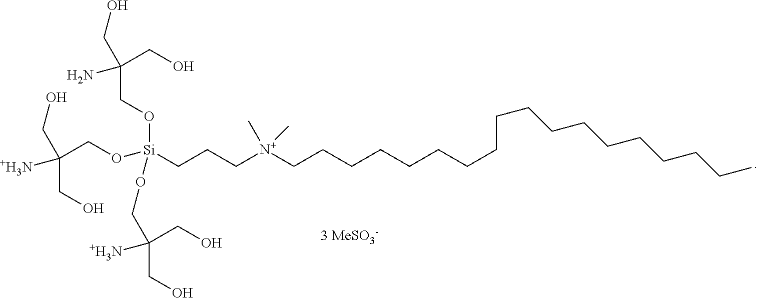 [SMILES: CCCCCCCCCCCCCCCCCC[N+](C)(C)CCC[Si](OCC(N)(CO)CO)(OCC([NH3+])(CO)CO)OCC([NH3+])(CO)CO]

In certain embodiments a compound is provided of structure

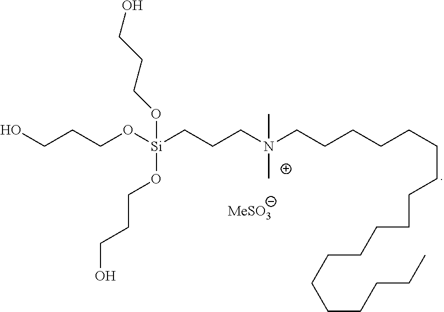

In certain embodiments a compound is provided of structure

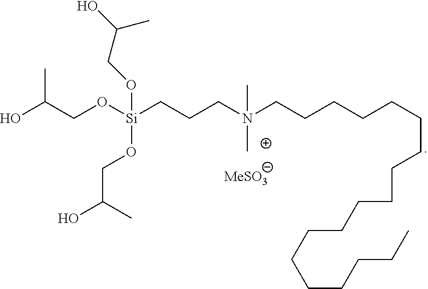

### Methods for the Treatment or Prevention of an Infection

The present invention provides methods for treating an infection in a host by administering an effective amount of an ammonium silane or composition described herein.

In some embodiments the infection is treated directly with a solution containing one or more ammonium silanes described herein (typically reconstituted from a sterilized powder or solid).

In certain embodiments, the infection to be treated with an ammonium silane or composition described herein is comprised of a biofilm.

In certain embodiments, the infection to be treated with an ammonium silane or composition described herein is an eye infection. Eye infections for treatment with an ammonium silane or composition described herein include red eyes, bacterial or viral conjunctivitis (pink eye), corneal ulcers, corneal keratitis, bacterial, fungal, herpal infectious keratitis, endophthalmitic, and blepharitis (lid infections). Conjunctivitis is the most common eye infection with viral conjunctivitis being the most common form. The most common causes of bacterial conjunctivitis are Haemophilus influenza, Streptococcus pneumoniae and Staphylococcus aureus. (Antibiotics versus placebo for acute bacterial conjunctivitis. Sheikh A, Hurwitz B., Cochrane Database Syst Rev. 2006 Apr. 19; (2):CD001211;) Both viral and bacterial conjunctivitis present with a red eye and are highly contagious. (https://www.ncbi.nlm.nih.gov/pmc/articles/PMC6003010/). In the subtropics and tropics, fungal keratitis is common, especially following a scratch to the cornea by vegetable matter. Fungal keratitis is the leading cost of unilateral blindness in the world. See, e.g., Brown et al., The global incidence and diagnosis of fungal keratitis. The Lancet, Volume 21, ISSUE 3, e49-e57, Mar. 1, 2021.

In one embodiment, the eye infection to be treated is blepharitis. Blepharitis is inflammation of the eyelids. It is a common cause of sore, red eyelids and crusty eyelashes. Lid margins can be infected due to bacterial or fungal infections, with accumulation forming a biofilm. Parasitic eyelash mites called Demodex feed on the biofilm, which in turn leads to an overgrowth of these mites that causes a worsening of the eyelid inflammation (https://www.allaboutvision.com/conditions/blepharitis.htm; https://www.reviewofoptometry.com/article/ro1117-could-eyelids-be-the-key-to-ded).

Bacterial infection is the most common cause of infectious keratitis. Common bacteria include S. aureus, coagulase-negative staphylococci, S. pneumoniae and Pseudomonas aeruginosa. (Sharma A, Taniguchi J. Review: Emerging strategies for antimicrobial drug delivery to the ocular surface: Implications for infectious keratitis. Ocul Surf 2017, 15, 670-9.; Green M, Apel A, Stapleton F. Risk factors and causative organisms in microbial keratitis. Cornea 2008, 27, 22-7). P. aeruginosa is the most common microorganism implicated in bacterial keratitis among contact lens wearers. Acanthamoeba is suspected if a patient has been swimming or in a spa while wearing contact lenses. (Stapleton F, Dart J K, Seal DV, Matheson M. Epidemiology of Pseudomonas aeruginosa keratitis in contact lens wearers. Epidemiol Infect 1995, 114, 395-402).

In one embodiment, the condition to be treated is dry eye syndrome. Dry eye syndrome or dry eye disease (DED) is one of the most common eye conditions worldwide. Often, dry eye is accompanied by an inflammation of the lid margins, and that may go relatively unnoticed as a cause rather than a result of dry eye. There are suggestive 4 stages of dry eye syndrome. Stage 1 involves the lash follicles, where a biofilm can establish itself. Stage 2 involves both the lash follicles and the meibomian glands and may explain obvious vs. non-obvious meibomian gland dysfunction (MGD). Because the biofilm blocks the large meibomian gland orifices (a combination of biofilm and poor or altered meibum). Stage 2 takes longer to achieve. Stage 3 involves the follicles, meibomian glands and the accessory lacrimal glands of Krause and Wolfring. The distance, narrow ducts and constant tear flushing serve to protect these glands for decades, making them the last glands affected by biofilm formation. Stage 4 occurs when the structural integrity of the eyelid finally breaks down due to the chronic inflammation, which can for example manifest clinically as lid laxity, floppy eyelid syndrome, ectropion and entropion. (Rynerson J M, Perry H D. DEBS-a unification theory for dry eye and blepharitis. Clin Ophthalmol. 2016, 10, 2455-67; Baudouin C. Ocular surface and external filtration surgery: mutual relationships. Dev Ophthalmol. 2012, 50, 64-78; Baudouin C, Messmer E M, Aragona P, et al. Revisiting the vicious circle of dry eye disease: a focus on the pathophysiology of meibomian gland dysfunction. Br J Ophthalmol. 2016, 100(3)300-6).

In one embodiment, the infection to be treated is an ear infection. Ear infections can occur in the outer ear canal (otitis externa), inner ear (otitis interna) or middle ear canal (otitis media). Most ear infections occur in the middle ear, when bacteria or virus grows, causing the accumulation of fluid, swelling, and inflammation. The infections can be chronic and generally due to biofilm accumulation. Recent clinical studies provide evidence that almost all chronic OM cases are accompanied by a bacterial biofilm behind the tympanic membrane (eardrum) and within the middle ear. Otitis media (OM) is the most common illness in children in the United States with 3/4 of children under the age of 3 having OM at least once. Even though most cases are chronic infections, long-term or permanent damage to the ear can still occur.

In some embodiments, the method of treatment of an ear infection involves application of a dressing comprising an effective amount of an ammonium silane, either alone or as a pharmaceutical composition, to the site of infection in a host in need thereof. The dressing is preferably shaped to fit within the space provided by the external auditory canal. The dressing may be malleable, allowing it to be compressed and shaped to fit within the external auditory canal, or it may be rigid, ensuring it will sit against the walls of the external auditory canal to ensure proper contact and transfer of the antimicrobial composition.

In some embodiments, the dressing is only placed in the external auditory canal for a day or less. In other embodiments, the dressing is placed in the external auditory canal for a week or more.

In some embodiments, the method of treatment of an infection in the ear canal of a host in need thereof involves application of the dressing into the ear canal of the host and subsequent saturation of the dressing with an ammonium silane or composition described herein. This method would allow for sequential or continuous application of the antimicrobial composition while the dressing is in place.

In some embodiments, the dressing is composed of a dissolvable material, requiring placement into the ear canal before saturation with the antimicrobial silane or composition. In another embodiment, the dressing is composed of polymeric foam that will expand upon subsequent wetting with the antimicrobial composition.

In one embodiment, the infection to be treated is a vaginal infection. Vulvovaginal candidiasis occurs when Candida species superficially penetrate the mucosal lining of the vagina and cause an inflammatory response. The dominant inflammatory cells are typically polymorphonuclear cells and macrophages. Patients may present with discharge, which is typically thick and adherent, or with excoriations, “external” dysuria, vaginal itching, vaginal burning, dyspareunia, or swelling. In some embodiments, the antimicrobial composition is formulated as a vaginal suppository.

In certain embodiments, the infection to be treated is an infection of vaginal mucosal tissue, for example endometritis. In certain embodiments, the vaginal infection comprises endometritis. In certain embodiments, the vaginal infection, for example endometritis is a bacterial infection caused by microorganisms or fungi selected from Streptococcus zooepidemicus, hemolytic Escherichia coli, Staphylococcus aureus, Candida, Aspergillus, or a combination thereof.

In some embodiments, the disorder to be treated is a periodontal, gum disease, or oral disorder. Bacteria in the oral cavity can cause gingivitis, periodontitis, dental caries, and endodontic abscesses. More than 1 in 5 people have untreated dental caries. Periodontal or gum disease is a pathological inflammatory condition of the gum and bone support (periodontal tissues) surrounding the teeth. The two most common periodontal diseases are gingivitis which is the inflammation of the gum at the necks of the teeth, and periodontitis which is inflammation affecting the bone and tissues of the teeth. Some oral disorders arise from repair of carious teeth wherein the cavity is prepared to receive restoration from an amalgam, gold or other restorative compound. The open cavity is treated with the antimicrobial preparation prior to completing the filling of the tooth in order to reduce the possibility of a biofilm infecting the dentin, producing more carious damage, and in order to strengthen the bond between filling material and the remaining dentin.

In some embodiments, the disorder to be treated is gingivitis. Gingivitis occurs in both chronic and acute forms. Acute gingivitis is usually associated with specific infections, micro-organisms, or trauma. Chronic inflammation of the gum tissue surrounding the teeth is associated with the bacterial biofilm (plaque) that covers the teeth and gums. (https://www.dentalhealth.ie/dentalhealth/causes/periodontaldisease.html).

In some embodiments, an ammonium silane or composition described herein is used to reduce or remove oral cavity microbial populations. In some embodiments, an ammonium silane or composition described herein is provided as a rinse, paste, gel, ointment, or pastille to aid in cleaning teeth, disinfecting cavities, removing plaque, eliminating harmful biofilms, and inhibiting re-infection. Oral cavity-linked bacterial species have been found in bacterial endocarditis, aspiration pneumonia, osteomyelitis, rheumatoid arthritis, and cardiovascular disease. Periodontitis has been associated with an increased risk of oral squamous cell carcinoma. Pathobiology 2021; 88:116-126, “Oral Microbiota and Cancer Development,” DOI: 10.1159/000510979. The main bacterial species found in the oral cavity is Streptococcus, Haemophilus, Lactobacillus acidophilus, Leptrotrichia, Porphyromonas gingivalis, Fusobacterium nucleatum, Prevotella, Propionibacterium, Staphylococcus Veillonella, and Treponema. Ninety-five percent of these bacteria exist as plaque biofilms. Bacterial biofilm infections remain prevalent reasons for implant failure. Fungal infections, Candida spp., papillomavirus (HPV) and Epstein-Barr virus can be involved in oncogeni mulation formation in the oral cavity leading to cancer development. Id. Pseudomembranous candidiasis is common in chronically ill patients and infants. P. gingivalis and F. nucleatum are able to release toxins that enable and maintain constant chronic inflammation. Polymicrobial oral biofilm communities to colonize in the presence of salivary and blood components. The papillated dorsal surface of the tongue and palatal mucosa beneath a maxillary denture are favored reservoir sites.

In some embodiments, an ammonium silane or composition described herein is used topically to treat an oral infection or sore. Topical application includes treatment of mouth/lip care, mouth ulcers and cold sores, including primary oral infection with the virus responsible for cold sores-herpes simplex virus (HSV). After the primary oral infection, HSV may remain inactive only to be activated later as the more common herpes labialis, or “cold sores”. Triggers for reactivation are well known and include sunlight, trauma, tiredness, stress, and menstruation. (https://www.dentalhealth.ie/dentalhealth/causes/coldsores.html) The most common form of mouth ulcer is called minor aphthous ulceration. Usually one to five small ulcers appear (less than 1 mm in diameter) on the inside of lips or cheeks, floor of the mouth or tongue. The ulcers tend to be concentrated towards the front of the mouth. Other more serious causes of mouth ulcers include herpes infection (https://www.dentalhealth.ie/dentalhealth/causes/mouthulcers.html).

In some embodiments, the infection to be treated using an ammonium silane or composition described herein is caused by a Candida species. Candida species, including the novel opportunistic pathogen Candida dubliniensis, are now emerging as major agents of nosocomial infections. Many such manifestations of infections associated with the formation of Candida biofilms include those occurring on devices such as indwelling intravascular catheters. Fungal biofilm-associated infections are frequently refractory to conventional therapy because of resistance to antimicrobial agents.

In some embodiments, the infection to be treated by an ammonium silane or composition described herein is composed of a pathogenic fungi. Pathogenic fungi can also adhere to abiotic surfaces such as prostheses and catheters; in particular, yeasts take advantage of this condition to gain access to blood circulation, reaching the internal organs of patients. This is alarming, as disseminated fungal infections have a high mortality rate. (Verstrepen K. J., Klis F. M. Flocculation, adhesion and biofilm formation in yeasts. Mol. Microbiol. 2006, 60, 5-15; doi: 10.1111/j.1365-2958.2006.05072.x)

In some embodiments, an ammonium silane or composition described herein targets a fungal biofilm. Candida albicans is the most studied model of biofilm formation and shows distinct phases of development that are similar to those of bacterial biofilms. Paracoccidioides brasiliensis is a dimorphic fungus responsible for paracoccidioidomycosis, a systemic mycosis endemic in Latin America. Sardi et al. (Sardi Jde C., Pitangui Nde S., Voltan A. R., Braz J. D., Machado M. P., Fusco Almeida A. M., Mendes Giannini M. J. In vitro Paracoccidioides brasiliensis biofilm and gene expression of adhesins and hydrolytic enzymes. Virulence. 2015, 6, 642-651. doi: 10.1080/21505594.2015.1031437.). Histoplasma capsulatum biofilm was first described by Pitangui et al. This fungus also features thermal dimorphism and is the cause of histoplasmosis, a respiratory and systemic mycosis whose evolution depends on the survival and replication of yeast in alveolar macrophages (Pitangui N. S., Sardi J. C., Silva J. F., Benaducci T., Moraes da Silva R. A., Rodriguez-Arellanes G., Taylor M. L., Mendes-Giannini M. J., Fusco-Almeida A. M. Adhesion of Histoplasma capsulatum to pneumocytes and biofilm formation on an abiotic surface. Biofouling. 2012, 28, 711-718. doi: 10.1080/08927014.2012.703659).

In some embodiments, an ammonium silane or composition described herein is used in treating an infection caused by a dermatophyte. Dermatophytes are fungi that invade keratinized tissues producing dermatophytosis, one of the most common dermatomycoses in human and animals (Weitzman I., Summerbell R. C. The dermatophytes. Clin. Microbiol. Rev. 1995, 8, 240-259. doi: 10.1016/50733-8635(05)70320-X). Among dermatophytosis, onychomycosis often relapses and involves long, sometimes ineffective treatment. Given this context and the hypothesis of Burkhart et al., which states that biofilm formation by dermatophytes can explain dermatophytomas, Costa-Orlandi et al., confirmed in vitro biofilm formation by two of the most prevalent species worldwide: Trichophyton rubrum and T. mentagrophytes (Burkhart C. N., Burkhart C. G., Gupta A. K. Dermatophytoma: Recalcitrance to treatment because of existence of fungal biofilm. J. Am. Acad. Dermatol. 2002, 47, 629-631. doi: 10.1067/mjd.2002.124699; Costa-Orlandi C. B., Sardi J. C., Santos C. T., Fusco-Almeida A. M., Mendes-Giannini M. J. In vitro characterization of Trichophyton rubrum and T. mentagrophytes biofilms. Biofouling. 2014, 30, 719-727. doi: 10.1080/08927014.2014.919282).

In some embodiments, an ammonium silane or composition described herein is used in treating an infection caused by Candida auris. Candida auris is an emerging yeast that causes healthcare-associated infections. It is the first Candida species to show resistance to all three major classes of antifungals. See, e.g., Sansom S, et al. Abstract 50. Presented at: Society for Healthcare Epidemiology of America Spring Meeting; Apr. 12-14, 2022. C. auris is also commonly found in biofilms on the body with other multidrug-resistant organisms, and is more commonly found with resistant gram-positive organisms such as MRSA and vancomycin-resistant Enterococcus. C. auris has been shown to be common on hospital surfaces. Id.

In another embodiment an infection is treated by applying a dressing comprising one or more ammonium silanes described herein as an antimicrobial composition to the site of infection, wherein the dressing releases one or more ammonium silanes described herein into the site of infection. The infection may involve the presence of bacteria, fungi, viruses, amoebas, or a combination of infectious species thereof.

In some embodiments, an ammonium silane or composition described herein is used in the treatment of a chronic wound. In some embodiments, the chronic wound is one in which the normal progression of healing has been stalled for over 4 weeks, with continued inflammation, exudation, and granulation tissue but without normal vascularization and epitheliazation.

In some embodiments, treatment of an infection comprises placing a dressing comprising an antimicrobial composition as described herein in or on the site of infection.

In another embodiment, treatment of an infection comprises placing an antimicrobial composition containing one or more ammonium silanes described herein at the site of infection. The length of time that the ammonium silane, or composition is applied is such that antimicrobial treatment is still effective or the infection has resolved. The treatment may be applied continuously, with concurrent successive applications after an appropriate time frame, or in alternation with another treatment for the infection after an appropriate time frame. An ammonium silane or composition described herein may be applied at the site of infection in a host at the appropriate interval as determined by a healthcare provider.

In some embodiments, an ammonium silane or composition described herein is placed at the site of infection for a day or less. In other embodiments, an ammonium silane or composition described herein is placed at a site of infection for a week or more. In other embodiments, an ammonium silane or composition described herein is placed on the chronic wound immediately after cleansing, irrigation, or other form of debridement at each visit in which the form of debridement is carried out.

An effective amount of an ammonium silane or composition as described herein, or an ammonium silane or composition described herein in combination or alternation with, or preceded by, concomitant with or followed by another active pharmaceutical agent, can be used in an amount sufficient to inhibit the progression of disorder, for example an infection, caused by the presence of an infectious organism in or on a host; cause a regression of a disorder caused by the presence of an infectious organism in or on a host; cause a cure of a disorder caused by the presence of an infectious organism in or on a host; or inhibit or prevent the development of a disorder caused by the presence of an infectious organism near, in or on a host. The method of treatment can be administered once a day (q.d.), twice a day (b.i.d.), three times a day (t.i.d.), four times a day (q.i.d.), once every other day (Q2d), once every third day (Q3d), as needed, or using any dosing schedule that provides treatment of an infection as described herein.

In some embodiments, the method of treating and/or preventing an infection comprises placing a dressing saturated with an ammonium silane or composition described herein in the site of a wound and/or infection. The dressing may be saturated with an ammonium silane or composition directly before placement into the site of a wound and/or infection or may be manufactured and packaged presaturated. In other embodiments, the method of treating and/or preventing an infection comprises placing a dressing in the site of a wound and/or infection followed by subsequent saturation of the dressing with an ammonium silane or composition. An ammonium silane or composition may be added after placement of the dressing by dropper, syringe, or other suitable means.

In some embodiments, one or more of ammonium silanes, or a pharmaceutical composition as described herein, are used to treat or to prevent a medical disorder which is mediated by the presence of a bacterium, for example a bacterial infection.

In some embodiments, an ammonium silane or composition described herein may be used to treat a disorder, typically an infection, caused by a gram-positive bacterium.

Non-limiting example of gram-positive bacteria which may be treated using the ammonium silanes described herein either alone or in combination with another therapeutic include: Actinomyces species including Actinomyces israelii, Actinomyces naeslundii, Actinomyces viscosus, Actinomyces odontolyticus, and Actinomyces pyogenes; Bacillus species including Bacillus anthracis, Bacillus cereus, and Bacillus subtilis; Clostridium species including Clostridium botulinum, Clostridium difficile, Clostridium perfringens, Clostridium sordellii, and Clostridium tetani; Corynebacterium species including Corynebacterium diphtheriae, Corynebacterium jeikeium, Corynebacterium minutissimum, Corynebacterium mucifaciens, Corynebacterium pseudotuberculosis, Corynebacterium striatum, Corynebacterium tenuis, and Corynebacterium ulcerans; Enterococcus species including Enterococcus casseliflavus, Enterococcus faecalis, Enterococcus faecium, Enterococcus raffinosus, and Enterococcus hirae; Leuconostoc species including Leuconostoc pseudomesenteroides; Micrococcus species such as Microccocus luteus; Nocardia species including Nocardia asteroids and Nocardia sienata; Propionibacterium species including Propionibacterium acnes; Staphylococcus species including Staphylococcus aureus, Staphylococcus capitis, Staphylococcus epidermidis, Staphylococcus haemolyticus, Staphylococcus hominis, Staphylococcus lugdunensis, Staphyloccocus pasteuri, and Staphyloccocus saprophyticus; and Streptococcus species including Streptococcus agalactiae, Streptococcus anginosus, Streptococcus bovis, Streptococcus dysgalactiae, Streptococcus mitis, Streptococcus mutans, Streptococcus pneumoniae, Streptococcus pyogenes, Streptococcus sanguinis, Streptococcus suis, and Streptococcus viridans.

In some embodiments, an ammonium silane or composition described herein may be used to treat a disorder, typically an infection, caused by a gram-negative bacterium.

Non-limiting examples of gram-negative bacteria which may be treated using the ammonium silanes described herein either alone or in combination with another therapeutic include: Acinetobacter species including Acinetobacter baumannii and Acinetobacter iwoffii; Aeromonas species including Aeromonas veronii biovar sobria (previously Aeromonas sobria), Aeromonas caviae, and Aeromonas hydrophila; Alcaligenes/Achromobacter species including Alcaligenes faecalis and Alcaligenes xylosoxidans; Bacteroides species including Bacteroides fragilis; Bartonella species including Bartonella bacillformis, Bartonella clarridgeiae, Bartonella elizabethae, Bartonella henselae, Bartonella koehlerae, Bartonalla naantalienis, Bartonella quintana, Bartonella rochalimae, Bartonella vinsonii, and Bartonella washoensis; Bordetella species including Bordetella bronchispetica, Bordetella pertussis, and Bordetella parapertussis; Borrelia species including Borrelia afzelii, Borrelia burgdorferi, Borrelia crocidurae, Borrelia duttoni, Borrelia garinii, Borrelia hermsii, Borrelia hispanica, Borellia miyamotoi, Borrelia parkeri, Borrelia persica, Borrelia recurrentis, Borrelia turicatae, and Borrelia venezuelensis; Brevundimonas species including Brevundimonas diminuta and Brevundimonas vesicularis; Brucella species including Brucella abortus, Brucella canis, Brucella melitensis, and Brucella suis; Burkholderia species including Burkholderia cepacia, Burkholderia mallei, and Burkholderia pseudomallei; Campylobacter species including Campylobacter jejuni, Campylobacter coli, Campylobacter upsaliensis, Campylobacter lari, and Campylobacter coli; Chlamydia/Chlamidophila species including Chlamydophila pneumoniae, Chlamydophila psittaci, Chlamidophila pecorum, and Chlamydia trachomatis; Citrobacter species including Citrobacter amalonaticus, Citrobacter freundii, Citrobacter koseri, and Citrobacter diversus; Coxiella burnetti; Ehrlichia species including Ehrlichia canis and Ehrlichia chaffeensis; Enterobacter species including Enterobacter aerogenes and Enterobacter cloacae; Escherichia species including Escherichia coli; Francisella species including Francisella novicida, Francisella philomiragia, and Francisella tularensis; Haemophilus species including Haemophilus influenzae and Haemophilus ducreyi; Helicobacter species including Helicobacter pylori; Klebsiella species including Klebsiella granulomatis, Klebsiella oxytoca, and Klebsiella pneumoniae; Leclercia adecarboxylata; Legionella species including Legionella pneumophila; Leptospira species including Leptospira interrogans, Leptospira noguchii, Leptospira santarosai, and Leptospira weilii; Listeria species including Listeria monocytogenes; Moraxella species including Moraxella catarrhalis, Moraxella lacunata, and Moraxella bovis; Moraxella bovoculi; Morganella species including Morganella morganii; Mycoplasma species including Mycoplasma amphoriforme, Mycoplasma buccale, Mycoplasma faucium, Mycoplasma fermentans, Mycoplasma genitalium, Mycoplasma hominis, Mycoplasma lipophilum, Mycoplasma orale, Mycoplasma penetrans, Mycoplasma pirum, Mycoplasma pneumoniae, Mycoplasma primatum, Mycoplasma salivarium, and Mycoplasma spermatophilum; Neisseria species including Neisseria meningitidis and Neisseria gonorrhoeae; Orientia species including Orientia tsutsugamushi and Orientia chuto; Pantoea species including Pantoea agglomerans; Paracoccus species including Paracoccus yeei; Prevotella species including Prevotella intermedia and Prevotella melaninogenica; Proteus species including Proteus mirabilis, Proteus penneri, and Proteus vulgaris; Providencia species including Providencia rettgeri and Providencia stuartii; Pseudomonas species including Pseucomonas aeruginoas, Pseudomonas oryzihabitans, Pseudomonas plecoglossidica, and Pseudomonas stutzeri; Ralstonia species including Ralstonia pickettii and Ralstonia insidiosa; Rickettsia species including Rickettsia africae, Rickettsia akari, Rickettsia australis, Rickettsia conorii, Rickettsiafelis, Rickettsia japonica, Rickettsia prowazekii, Rickettsia rickettsia, Rickettsia sibirica, and Rickettsia typhi; Roseomonas species including Roseomonas gilardii; Salmonella species including Salmonella bongori, Salmonella enterica, Salmonella paratyphi, Salmonella typhi, and Salmonella typhimurium; Serratia species including Serratia marcescens, Serratia liquefaciens, Serratia rubidaea, and Serratia odoriferae; Shigella species including Shigella dysenteriae and Shigella sonnei; Sphingomonas species including Sphingomonas mucosissima and Sphingomonas paucimobilus; Stenotrophomas species including Stenotrophomas maltophilia; Treponema species including Treponema carateum, Treponema paraluiscuniculi, and Treponema pallidum; Ureaplasma species including Ureaplasma urealyticum; Vibrio species including Vibrio cholera, Vibrio parahaemolyticus, and Vibrio vulnificus; and Yersinia species including Yersinia enterocolitica, Yersinia pestis, and Yersinia pseudotuberculosis.

Non-limiting examples of disorders mediated by a bacterium that may be treated by an ammonium silanes or compositions described herein, either alone or in combination with another therapeutic, include actinomycosis, anaplasmosis, anthrax, bacillary angiomatosis, actinomycetoma, bacterial conjunctivitis, bacterial keratitis, bacterial pneumonia, bacterial vaginosis, bacterial endocarditis, bartonellosis, botulism, boutenneuse fever, brucellosis, bejel, brucellosis spondylitis, bubonic plague, Buruli ulcer, Bairnsdale ulcer, bacillary dysentery, campylobacteriosis, Carrion's disease, cat-scratch disease, cellulitis, chancroid, chlamydia, chlamydia conjunctivitis, clostridial myonecrosis, cholera, Clostridium difficile colitis, diphtheria, Daintree ulcer, donavanosis, dysentery, erhlichiosis, epidemic typhus, fried rice syndrome, five-day fever, floppy baby syndrome, Far East scarlet-like fever, gas gangrene, glanders, gonorrhea, granuloma inguinale, human necrobacillosis, necrotizing fasciitis, hemolytic-uremic syndrome, human ewingii ehrlichiosis, human monocytic ehrlichiosis, human granulocytic anaplasmosis, infant botulism, Izumi fever, Kawasaki disease, Kumusi ulder, lymphogranuloma venereum, Lemierre's syndrome, Legionellosis, leprosy, leptospirosis, listeriosis, Lyme disease, lymphogranuloma venereum, Malta fever, Mediterranean fever, myonecrosis, mycoburuli ulcer, mucocutaneous lymph node syndrome, meliodosis, meningococcal disease, murine typhus, Mycoplasma pneumonia, mycetoma, neonatal conjunctivitis, nocardiosis, Oroya fever, ophthalmia neonatorum, ornithosis, Pontiac fever, peliosis hepatis, pneumonic plague, postanginal shock including sepsis, pasteurellosis, pelvic inflammatory disease, pertussis, plague, pneumococcal infection, pneumonia, psittacosis, parrot fever, pseudotuberculosis, Q fever, quintan fever, rabbit fever, relapsing fever, rickettsialpox, Rocky Mountain spotted fever, rat-bite fever, Reiter syndrome, rheumatic fever, salmonellosis, scarlet fever, sepsis, septicemic plague, Searls ulcer, shigellosis, soft chancre, syphilis, streptobacillary fever, scrub typhus, Taiwan acute respiratory agent, Trench fever, trachoma, tuberculosis, tularemia, typhoid fever, typhus, tetanus, toxic shock syndrome, undulant fever, ulcus molle, Vibrio parahaemolyticus enteritis, Whitmore's disease, walking pneumonia, Waterhouse-Friderichsen syndrome, yaws, and yersiniosis.

In some embodiments, ammonium silanes described herein may be used to treat a disorder, typically an infection, caused by a mycobacterium.

Non-limiting examples of mycobacteria which may be treated using an ammonium silanes described herein either alone or in combination with another therapeutic include Mycobacterium abcessus, Mycobacterium africanum, Mycobacterium agri, Mycobacterium aichiense, Mycobacterium alvei, Mycobacterium arabiense, Mycobacterium aromaticivorans, Mycobacterium arosiense, Mycobacterium arupense, Mycobacterium aquaticum, Mycobacterium asiaticum, Mycobacterium aubagnese, Mycobacterium aurum, Mycobacterium austroafricanum, Mycobacterium avium, Mycobacterium avium paratuberculosis, Mycobacterium avium silvaticum, Mycobacterium avium hominussuis, Mycobacterium bacteremicum, Mycobacterium barrassiae, Mycobacterium boenickei, Mycobacterium bohemicum, Mycobacterium bolletii, Mycobacterium botniense, Mycobacterium bovis, Mycobacterium branderi, Mycobacterium brisbanense, Mycobacterium brumae, Mycobacterium canariasense, Mycobacterium canettii, Mycobacterium caprae, Mycobacterium chimaera, Mycobacterium chelonae, Mycobacterium chitae, Mycobacterium chubuense, Mycobacterium colombiense, Mycobacterium conceptionense, Mycobacterium confluentis, Mycobacterium conspicuum, Mycobacterium cookii, Mycobacterium cosmeticum, Mycobacterium diernhoferi, Mycobacterium doricum, Mycobacterium duvali, Mycobacterium elephantis, Mycobacterium fallax, Mycobacterium farcinogenes, Mycobacterium flavescens, Mycobacterium florentinum, Mycobacterium fortuitum, Mycobacterium frederikbergense, Mycobacterium gadium, Mycobacterium gastri, Mycobacterium genavense, Mycobacterium gilvum, Mycobacterium gordonae, Mycobacterium haemophilum, Mycobacterium hassiacum, Mycobacterium heidelbergense, Mycobacterium heckshornense; Mycobacterium hiberniae, Mycobacterium hodleri, Mycobacterium holsaticum, Mycobacterium houstonense, Mycobacterium icosiumassilensis, Mycobacterium immunogenum, Mycobacterium indicus pranii, Mycobacterium intacellulare, Mycobacterium intracellulare, Mycobacterium interjectum, Mycobacterium intermedium, Mycobacterium iranicum, Mycobacterium kansasii, Mycobacterium komossense, Mycobacterium kubicae, Mycobacterium lentiflavum, Mycobacterium leprae, Mycobacterium lepraemurium, Mycobacterium lepromatosis, Mycobacterium liflandii, Mycobacterium llatzerense, Mycobacterium madagascariense, Mycobacterium mageritense, Mycobacterium malmoense, Mycobacterium marinum, Mycobacterium massiliense, Mycobacterium massilipolynesiensis, Mycobacterium microti, Mycobacterium monacense, Mycobacterium montfiorense, Mycobacterium morokaense, Mycobacterium mucogenicum, Mycobacterium mungi, Mycobacterium murale, Mycobacterium nebraskense, Mycobacterium neoaurum, Mycobacterium neworleansense, Mycobacterium nonchromogenicum, Mycobacterium obuense, Mycobacterium orygis, Mycobacterium palustre, Mycobacterium parascofulaceum, Mycobacterium parafortuitum, Mycobacterium perigrinum, Mycobacterium phlei, Mycobacterium phocaicum, Mycobacterium pinnipedii, Mycobacterium porcinum, Mycobacterium pseudoshottsii, Mycobacterium psychotolerans, Mycobacterium pulveris, Mycobacterium pyrenivorans, Mycobacterium saskatchewanense, Mycobacterium sediminis, Mycobacterium senegalense, Mycobacterium septicum, Mycobacterium shimoidei, Mycobacterium shottsii, Mycobacterium simiae, Mycobacterium smegmatis, Mycobacterium sphagni, Mycobacterium stephanolepidis, Mycobacterium suricattae, Mycobacterium szulgai, Mycobacterium talmoniae, Mycobacterium terrae, Mycobacterium thermoresistibile, Mycobacterium triplex, Mycobacterium triviale, Mycobacterium tuberculosis, Mycobacterium tusciae, Mycobacterium ulcerans, Mycobacterium vaccae, Mycobacterium vanbaalenii, Mycobacterium xenopi, and Mycobacterium yongonense.

In some embodiments, one or more of the ammonium silanes, or pharmaceutical composition as described herein, are used to treat or to prevent a medical disorder which is mediated by the presence of a fungus or algae, for example a fungal infection.

Non-limiting examples of algae and fungi which may be treated using ammonium silanes described herein either alone or in combination with another therapeutic include: Absidia species including Absidia corymbifera; Alterania species including Alterania alternate; Aspergillus species including Aspergillus clavatus, Aspergullus flavus, Aspergillus fumigatus, Aspergillus niger, Aspergillus sydowii, Aspergillus terreus, Aspergillus versicolor, and Aspergillus verrucaria; Aureobasidium species including Aureobasidium pullans; Batrachochytrium species including Batrachochytrium dendrobatidis and Batrachochytrium salamandrivorans; Blastomyces species including Blastomyces dermatitidis; Candida species including Candida albicans, Candida auris, Candida dubliniensis, Candida glabrata, Candida parapsilosis, Candida rugosa, and Candida tropicalis; Chaetomium species including Chaetomium globsum; Cladosporium species including Cladosporium cladosporoides; Chlorophyta, Coccidioides species including Coccidioides immitis and Coccidioides posadasii; Cryptococcus species including Cryptococcus albidus, Cryptococcus gattii, Cryptococcus laurentii, Cryptococcus neoformans, and Cryptococcus uniguttulatus; Cunninghamella species; Curvularia species including Curvularia brachyspora, Curvularia clavata, Curvularia geniculata, Curvularia lunata, Curvularia pallescens, Curvularia senegalensis, and Curvularia verruculosa; Cyanophyta Chrysophyta, Dreschslera species including Dreschlera australiensis; Epidermophyton species including Epidermophyton floccosum; Fonsecaea species including Fonsecaea compacta and Fonsecaea pedrosoi; Fusarium species including Fusarium solani, Fusarium oxysporum, and Fusarium chlamydosporum, Geotrichum species including Geotrichum capitatum, Geotrichum candidum, and Geotricum clavatum; Gliomastix species including Gliomastix cerealis; Gloeophyllum species including Gloeophyllum trabeum; Histoplasma species including Histoplasma capsulatum and Histoplasma capsulatum var. faciminosum; Malassezia species including Malassezia furfur and Malassezia globosa; Microsporum species; Monilia species including Monilia grisea; Mucor species including Mucor indicus; Paracoccidioides species including Paracoccidioides brasiliensis; Penicillium species; Piedraia species including Piedraia hortae and Piedraia quintanilhae; Phialophora species including Phialophora verrucosa; Phoma species including Phoma fimeti; Pithomyces species including Pithomyces chartarum; Pneumocystis species including Pneumocystis carinii and Pneumocystis jirovecii; Poria species including Poria placenta; Rhizopus species including Rhizopus microspores, Rhizopus oryzae, and Rhizopus stolonfer; Scolecobasidium species including Scolecobasidium humicola; Sporothrix species including Sporothrix brasiliensis, Sporothrix globosa, and Sporothrix schenckii; and Trichoderma species including Trichoderma viride; and Trichophyton species including Trichosporon beigeli, Trichophyton concentricum, Trichophyton interdigitale, Trichophyton mentagrophytes, Trichophyton rubrum, and Trichophyton tonsurans.

Non-limiting examples of disorders mediated by a fungus that may be treated byammonium silanes described herein, either alone or in combination with another therapeutic, include invasive aspergillosis, black piedra, blastomycosis, oropharyngeal candidiasis, vulvovaginal candidiasis, chromoblastomycosis, chytridiomycosis, coccidioimycosis, cryptococcosis, dermatophytosis, fusariosus, geotrichosis, histoplasmosis, mucormycosis, mycetoma, paracoccidioidomycosis, pneumocystis pneumonia, sporotrichosis, Tinea barbae, Tinea capitis, Tinea corporis, Tinea cruris, Tinea manum, Tinea nigra, Tinea unguium, Tinea versicolor, white piedra, and zygomycosis.

In some embodiments, one or more of the ammonium silanes, or pharmaceutical composition as described herein, are used to treat or to prevent a medical disorder which is mediated by the presence of amoeba, for example an amoebal infection.

Non-limiting examples of amoeba which may be treated using ammonium silanes described herein either alone or in combination with another therapeutic include: Acanthamoeba species; Balamuthia species including Balamuthia mandrillaris; Dientamoeba species including Dientamoeba fragilis; Endolimax species including Endolimax nana; Entamoeba species including Entamoeba Bangladeshi, Entamoeba coli, Entamoeba dispar, Entamoeba gingivalis, Entamoeba hartmanni, Entamoeba histolytica, Entamoeba moshkovskii, and Entamoeba polecki; Iodamoeba species including Iodamoeba butschlii; Naegleria species including Naegleriafowleri; and Sappinia species including Sappinia diploidea and Sappinia pedata.

Non-limiting examples of disorders mediated by an amoeba that may be treated by ammonium silanes described herein, either alone or in combination with another therapeutic, include amoebiasis, amoebic dysentery, amoebic liver abscess, cutaneous amoebiasis, amoebic brain abscess, amebiasis cutis, Acanthamoeba keratitis, cutaneous acanthamoebiasis, granulomatous amoebic encephalitis, Balamuthia amoebic encephalitis, and Sappinia amoebia encephalitis.

In some embodiments, the infection is caused by Acinetobacter species, Aspergillus species, Burkholderia cepacia complex, Campylobacter species, Candida species, Clostridium difficile, Coccidioides species, Cryptococcus species, Enterobacteriaceae, Enterococcus species, Helicobacter pylori, Mycobacterium tuberculosis complex, Neisseria gonorrhoeae, Neisseria meningitidis, Non-tuberculous mycobacteria species, Pseudomonas species, Staphylococcus aureus, Streptococcus agalactiae, Streptococcus pneumoniae, Streptococcus pyogenes, and Vibrio cholerae.

In certain alternative embodiments, the infection is caused by Staphylococcus aureus. Staphylococcus epidermidis, Pseudomonas aeruginosa, Streptococcus pyogenes, Candida albicans, Candida auris, Cladosporium herbarum, Aspergillus niger, Proteus mirabilis, Klebsiella pneumoniae, Acinetobacter baumanii, an Enterobacter spp. or a Fusarium spp.

In some embodiments, an ammonium silane or composition described herein may be used to treat an inflammatory disorder caused by the presence of an infectious organism, for example a bacterium, a fungus, a virus, or an amoeba as described herein.

Non-limiting examples of such inflammatory disorders include adenoiditis, appendicitis, arteritis, ascending cholangitis, balanitis, blepharitis, bronchitis, bursitis, cellulitis, cerebral vasculitis, cervicitis, chemosis, cholecystitis, chondritis, choroioamnionitis, colitis, conjunctivitis, constrictive pericarditis, cryptitis, dacryoadenitis, dermatitis, diabetic ulcer, duodenal lymphocytosis, encephalitis, endocarditis, endometritis, endotheliitis, enteritis, enterocolitis, eosinophilis fasciitis, epididymitis, esophagitis, folliculitis, gastritis, gingivitis, glomerulonephritis, glossitis, hepatitis, infectious arthritis, ileitis, intertrigo, keratitis, keratoconjunctivitis, labyrithitis, lymphadenitis, mastitis, mastoiditis, myocarditis, myopericarditis, myositis, necrotizing fasciitis, nephritis, omaphalitis, oophoritis, ophthalmitis, orchitis, osteitis, osteomyelitis, pancreatitis, paraproctitis, parotitis, pericarditis, perichondritis, perifolliculitis, periodontitis, peritonitis, pharyngitis, phlebitis, pleurisy, pneumonitis, pulmonitis, proctitis, prostatitis, pulpitis, pyelonephritis, pyomyositis, retinal vasculitis, rheumatic fever, rhinitis, scleritis, salpingitis, sialadenitis, sinusitis, stomatitis, synovitis, septicemia, tenosynovitis, thyroiditis, tonsillitis, tularemia, urethritis, uveitis, vaginitis, vasculitis, and vulvitus.

In some embodiments, an ammonium silane or composition described herein is used to treat a skin infection in a host, for example a human. The infection may be caused by a bacterium, a fungus, an amoeba, or a virus as described herein.

In another embodiment, the method is used to treat a skin infection in another mammal, for example a cat, a dog, a cow, a pig, or a horse.

Examples of bacterial cutaneous infections that may be treated by an ammonium silane or composition described herein include, but are not limited to: acne vulgaris, African tick bite fever; American tick bite fever (Rickettsia parkeri infection); Bacillary angiomatosis; Bejel (endemic syphilis); Blastomycosis-like pyoderma (pyoderma vegetans); Blistering distal dactylitis; Botryomycosis; Brill-Zinsser disease; Brucellosis (Bang's disease, Malta fever, undulant fever); Bubonic plague; Bullous impetigo; Campylobacter jejuni; Cat scratch disease (cat scratch fever, English-Wear infection, inoculation lymphoreticulosis, subacute regional lymphadenitis); Cellulitis; Chancre; Chancroid (soft chancre, ulcus molle); Chronic lymphangitis; Chronic recurrent erysipelas; Chronic undermining burrowing ulcers (Meleney gangrene); Condylomata lata; Cutaneous actinomycosis; Dermatitis gangrenosa (gangrene of the skin); Ecthyma; Ecthyma gangrenosum; Elephantiasis nostras; Endemic typhus (murine typhus); Endometritis; Epidemic typhus (epidemic louse-borne typhus); Erysipelas (ignis sacer, Saint Anthony's fire); Erysipeloid of Rosenbach; Erythema marginatum; Erythrasma; Felon; Flea-borne spotted fever; Flinders Island spotted fever; Flying squirrel typhus; Folliculitis; Fournier gangrene (Fournier gangrene of the penis or scrotum); Furunculosis (boil); Gas gangrene (clostridial myonecrosis, myonecrosis); Glanders (equinia, farcy, malleus); Gonococcemia (arthritis-dermatosis syndrome, disseminated gonococcal infection); Gonorrhea (clap); Gram-negative folliculitis; Gram-negative toe web infection; Granuloma inguinale (Donovanosis, granuloma genitoinguinale, granuloma inguinale tropicum, granuloma venereum, granuloma venereum genitoinguinale, lupoid form of groin ulceration, serpiginous ulceration of the groin, ulcerating granuloma of the pudendum, ulcerating sclerosing granuloma); Green nail syndrome; Hospital furunculosis; Hot tub folliculitis (Pseudomonas aeruginosa folliculitis); Human granulocytotropic anaplasmosis; Human monocytotropic ehrlichiosis; Impetigo contagiosa; Japanese spotted fever; Leptospirosis (Fort Bragg fever, pretibial fever, Weil's disease); Listeriosis; Ludwig's angina; Lupoid sycosis; Lyme disease (Afzelius' disease, Lyme borreliosis); Lymphogranuloma venereum (climatic bubo, Durand-Nicolas-Favre disease, lymphogranuloma inguinale, poradenitis inguinale, strumous bubo); Malakoplakia (malacoplakia); Mediterranean spotted fever (Boutonneuse fever); Melioidosis (Whitmore's disease); Meningococcemia; Missouri Lyme disease; Necrotizing fasciitis (flesh-eating bacteria syndrome); Neonatal toxic shock-like exanthematous disease; Noma neonatorum; North Asian tick typhus; Ophthalmia neonatorum; Oroya fever (Carrion's disease); Perianal cellulitis (perineal dermatitis, streptococcal perianal disease); Periapical abscess; Persistent mating-induced endometritis (PMIE); Pinta; Pitted keratolysis (keratolysis plantare sulcatum, keratoma plantare sulcatum, ringed keratolysis); Plague; Primary gonococcal dermatitis; Pseudomonal Pyoderma; Pseudomonas hot-foot syndrome; Pyogenic paronychia; Pyomyositis; Q fever; Queensland tick typhus; Rat-bite fever; Recurrent toxin-mediated perineal erythema; Rhinoscleroma; Rocky Mountain spotted fever; Scarlet fever; Scrub typhus (Tsutsugamushi fever); Shigellosis; Staphylococcal scalded skin syndrome (pemphigus neonatorum, Ritter's disease); Streptococcal intertrigo; Superficial pustular folliculitis (impetigo of Bockhart, superficial folliculitis); Sycosis vulgaris (barber's itch, sycosis barbae); Syphilid; Syphilis (lues); Tick-borne lymphadenopathy; Toxic shock syndrome (streptococcal toxic shock syndrome, streptococcal toxic shock-like syndrome, toxic streptococcal syndrome); Trench fever (five-day fever, quintan fever, urban trench fever); Tropical ulcer (Aden ulcer, jungle rot, Malabar ulcer, tropical phagedena); Tularemia (deer fly fever, Ohara's disease, Pahvant Valley plague, rabbit fever); Verruga peruana; and Yaws (bouba, frambosie, parangi, pian).

Examples of mycobacterial cutaneous infections that may be treated by an ammonium silane or composition described herein include, but are not limited to: Aquarium granuloma (fish-tank granuloma, swimming-pool granuloma); Borderline lepromatous leprosy; Borderline leprosy; Borderline tuberculoid leprosy; Buruli ulcer (Bairnsdale ulcer, Searl ulcer, Searle's ulcer); Erythema induratum (Bazin disease); Histoid leprosy; Lepromatous leprosy; Leprosy (Hansen's disease); Lichen scrofulosorum (tuberculosis cutis lichenoides); Lupus vulgaris (tuberculosis luposa); Miliary tuberculosis (disseminated tuberculosis, tuberculosis cutis acuta generalisata, tuberculosis cutis disseminata); Papulonecrotic tuberculid; Primary inoculation tuberculosis (cutaneous primary complex, primary tuberculous complex, tuberculous chancre); Scrofuloderma (tuberculosis cutis colliquativa); Tuberculosis cutis orificialis (acute tuberculous ulcer, orificial tuberculosis); Tuberculosis verrucosa cutis (lupus verrucosus, prosector's wart, warty tuberculosis); Tuberculous cellulitis; Tuberculous gumma (metastatic tuberculous abscess, metastatic tuberculous ulcer); and Tuberculoid leprosy.

Examples of fungal cutaneous infections that may be treated by an ammonium silane or composition described herein include, but are not limited to: African histoplasmosis; Alternariosis; Antibiotic candidiasis (iatrogenic candidiasis); Black piedra; Candida auris, Candidal intertrigo; Candidal onychomycosis; Candidal paronychia; Candidal vulvovaginitis; Candidid; Chromoblastomycosis (chromomycosis, cladosporiosis, Fonseca's disease, Pedroso's disease, phaeosporotrichosis, verrucous dermatitis); Chronic mucocutaneous candidiasis; Coccidioidomycosis (California disease, desert rheumatism, San Joaquin Valley fever, valley fever); Congenital cutaneous candidiasis; Cryptococcosis; Dermatophytid; Diaper candidiasis; Disseminated coccidioidomycosis (coccidioidal granuloma); Distal subungual onychomycosis; Entomophthoromycosis; Erosio interdigitalis blastomycetica; Favus; Fungal folliculitis (majocchi granuloma); Fusariosis; Geotrichosis; Granuloma gluteale infantum; Histoplasmosis (cave disease, Darling's disease, Ohio Valley disease, reticuloendotheliosis); Hyalohyphomycosis; Kerion; Lobomycosis (keloidal blastomycosis, lacaziosis, Lobo's disease); Mucormycosis; Mycetoma (Madura foot, maduromycosis); North American blastomycosis (blastomycetic dermatitis, blastomycosis, Gilchrist's disease); Onychomycosis (dermatophytic onychomycosis, ringworm of the nail, tinea unguium); Oral candidiasis (thrush); Otomycosis; Perianal candidiasis; Perleche (angular cheilitis); Phaeohyphomycosis; Piedra (trichosporosis); Pityrosporum folliculitis; Primary cutaneous aspergillosis; Primary cutaneous coccidioidomycosis; Primary cutaneous histoplasmosis; Primary pulmonary coccidioidomycosis; Primary pulmonary histoplasmosis; Progressive disseminated histoplasmosis; Proximal subungual onychomycosis; Rhinosporidiosis; South American blastomycosis (Brazilian blastomycosis, paracoccidioidal granuloma, paracoccidioidomycosis); Sporotrichosis (rose-gardener's disease); Systemic candidiasis; Tinea barbae (barber's itch, ringworm of the beard, tinea sycosis); Tinea capitis (herpes tonsurans, ringworm of the hair, ringworm of the scalp, scalp ringworm, tinea tonsurans); Tinea corporis (ringworm, tinea circinata, tinea glabrosa); Tinea corporis gladiatorum; Tinea cruris (crotch itch, eczema marginatum, gym itch, jock itch, ringworm of the groin); Tinea faciei; Tinea imbricate (tokelau); Tinea incognito; Tinea manuum; Tinea nigra (superficial phaeohyphomycosis, tinea nigra palmaris et plantaris); Tinea pedis (athlete's foot, ringworm of the foot); Tinea versicolor (dermatomycosis furfuracea, pityriasis versicolor, tinea flava); White piedra; White superficial onychomycosis; and Zygomycosis (phycomycosis).

### Wounds

A particular aspect provided herein is the use of an ammonium silane or composition described herein for the treatment of a wound or burn on a human or an animal. A compound or composition described herein can be contained in a wash, lotion, ointment, liquid, gel, film or other type of carrier for direct application to the wound. In an alternative aspect, a compound or composition described herein can be contained in a dressing, foam, wrap, bandage, gauze, film, packing, or other material as described above, wherein the material is applied to or used to cover and moisturize the wound or burn. In some embodiments, a compound described herein is contained in a material applied to or used to cover the wound or burn and is released into the wound, for example, in a controlled release manner.

Types of wounds that can be treated include a chronic wound, for example but not limited to, a pressure ulcer, venous ulcer, arterial wound, neuropathic ulcer, diabetic ulcer, for example a lower limbic ulcer or foot ulcer, skin tear, or moisture-associated skin damage (MASD), for example incontinence-associated dermatitis. In some embodiments, the compounds described herein are used to treat a wound caused by a burn.

Pressure ulcers (also known as pressure injuries) are defined by the National Pressure Ulcer Advisory Panel (NPUAP) as localized damage to the skin and/or underlying soft tissue, usually over a bony prominence or related to a medical or other device. The injury can present as intact skin or an open ulcer and may be painful. The injury results from intense and/or prolonged pressure or pressure combined with shear. The tolerance of soft tissue for pressure and shear may also be affected by microclimate, nutrition, perfusion, comorbidities, and condition of the soft tissue. Pressure ulcers are described according to the NPUAP staging system based on damage that is clinically observed.

Venous ulcers are related to incompetence of the valves of the lower extremities, allowing blood to reflux into the superficial venous system and causing edema. Incomplete emptying of the deep veins can result in higher-than-normal pressure in the peripheral venous system of the lower extremities, which can eventually result in ulcerations.

Arterial wounds result from severe tissue ischemia. One of the most common causes of lower extremity arterial disease and ulceration is atherosclerosis of peripheral arterial vessels. Diabetic foot wounds are also called neuropathic ulcers. Peripheral neuropathy is present in over 80% of patients with foot ulcers. Neuropathy promotes ulcer formation by altering both pain sensation and pressure perception in the foot. Neuropathy can also alter the microcirculation and impair skin integrity. Once wounds occur, healing may be difficult to achieve, especially in patients with deep tissue or bone infections and in those with diminished blood flow to the foot.

A skin tear is defined by the International Skin Tear Advisory Panel (ISTAP) as a traumatic wound caused by mechanical forces (shear, friction, or blunt force), such as the mechanical force required to remove adhesives. Severity may vary by depth but does not extend through the subcutaneous layer. Skin tears are classified based on the degree of skin damage: Type 1: no skin loss; a skin flap can be positioned to cover the exposed wound base; Type 2: partial loss of the skin flap; Type 3: total loss of the skin flap; entire wound bed is exposed.

Moisture-associated skin damage (MASD) is defined as the inflammation and erosion of the skin that accompanies exposure to many different types of corrosive moisture, such as urine, perspiration, and wound drainage. Chronic exposure to moisture macerates the skin, impairing its protective mechanisms and disrupting normal skin flora, which can predispose the patient to cutaneous infections such as candidiasis. Incontinence-associated dermatitis (IAD), a subtype of MASD, is caused by chronic exposure to urine and/or liquid stool.

### Acne Vulgaris

A particular aspect provided herein is the use of an ammonium silane or composition described herein for the treatment of acne. Acne is a common skin disease that occurs when hair follicles become clogged with dead skin cells and oil from the skin. Acne vulgaris severity may be classified as mild, moderate, or severe. Mild acne is classically defined by the presence of clogged skin follicle (known as comedones) limited to the face with occasional inflammatory lesions. Moderate acne occurs when a higher number of inflammatory papules and pustules occur on the face, with some being found on the trunk of the body. Severe acne occurs when nodules are the characteristic facial lesions and involvement of the trunk is extensive.

Typically, acne is caused by colonization of the follicle and excessive outgrowth of Propionibacterium acnes bacteria and inflammation induced in response to the P. acnes bacteria. While less common, Staphylococcus epidermidis can also cause acne. The earliest pathological change involves the formation of a microcomedone due to the accumulation of skin cells in the hair follicle, which mix with oily sebum to block the follicle, a process further exacerbated by the presence of the P. acnes biofilm. If the microcomedone is superficial, melanin within the plug oxidizes upon exposure to air, forming a blackhead or open comedo. If the microcomedone is deeper within the hair follicle, a whitehead or closed comedo forms.

Propionibacterium acnes (reclassified as Cutibacterium acnes in 2016) is a Gram-positive bacterium (rod) linked to acne that belongs to the Cutibacterium Genus and the Propionibacteriaceae Family. Typically, slow-growing, it is aerotolerant anaerobe, meaning that it can tolerate the presence of oxygen, but does not utilize oxygen for its growth. While the bacteria are involved in the maintenance of healthy skin, it can also cause many common skin disorders such as acne vulgaris. The bacteria predominately live deep within follicles and pores, where it uses sebum, cellular debris, and metabolic byproducts from surrounding skin tissue as a source of energy and nutrients. Elevated production of sebum or blockage of follicles can cause the bacteria to grow and this rapid growth can trigger inflammation that can lead to the symptoms of common skin disorders, such as folliculitis and acne vulgaris.

The presence of P. acnes induces skin inflammation due to the bacteria's ability to bind to toll-like receptors (TLRs), especially TLR2 and TLR4 and by altering the fatty composition of the oily sebum by oxidizing squalene. The subsequent inflammatory cascades lead to the formation of inflammatory acne lesions such as papules, pustules, or nodules. If the inflammatory reaction is very severe, the follicle will break into the dermis and subcutaneous tissue as a deep nodule, leading to local tissue destruction and scarring.

In certain embodiments, the compounds described herein may be used in the topical compositions and methods provided herein, having anti-microbial effects that help alleviate the symptoms of acne vulgaris and treats the underlying overgrowth of bacterial that cause acne, for example, the bacterium Propionibacterium acnes or Staphylococcus epidermidis. In certain embodiments, the compounds provided herein, may be applied in topical formulation in any amount that achieves the desired effect. In certain non-limiting examples, the weight percentage of the active compound in the topical formulation is from about 0.1% to about 50%, or from about 0.1% to about 40%, or about 1% to about 30%, or from about 2, 3, 4 or 5% to about 20%, or between about 5% to about 10%. Examples include at least about 0.5, 1, 2, 3, 4, 5, 10 or 15% by weight.

In certain embodiments, treatment of an infection comprises placing the topical composition containing one or more ammonium silanes described herein at the site of infection. The length of time that the ammonium silane, or composition is applied is such that antimicrobial treatment is still effective or the infection has resolved. The treatment may be applied continuously, with concurrent successive applications after an appropriate time frame, or in alternation with another treatment for the infection after an appropriate time frame. The ammonium silanes described herein may be applied at the site of infection in a host at the appropriate interval as determined by a healthcare provider.

In some embodiments, the ammonium silanes described herein are placed at the site of infection for a day or less. In other embodiments, the ammonium silanes described herein are placed at a site of infection for a week or more.

In certain embodiments, the method of treatment can be administered once a day (q.d.), twice a day (b.i.d.), three times a day (t.i.d.), four times a day (q.i.d.), once every other day (Q2d), once every third day (Q3d), as needed, or using any dosing schedule that provides treatment of an infection as described herein.

In certain embodiments, a method is provided for the treatment of acne vulgaris in a human comprising administering a topical formulation containing, either alone or in combination with an effective amount of an antibiotic.

In certain embodiments, a topical formulation is also provided for the treatment of acne vulgaris comprising administering an effective amount of a compound described herein, or its pharmaceutically acceptable salt and a topically acceptable carrier.

In certain embodiments, the compounds described herein can be administered to a human in need thereof as a neat chemical, but are more commonly administered as a topical formulation that includes an effective amount of a compound described herein, or its pharmaceutically acceptable salt, for a human in need of treatment of acne vulgaris.

### Eczema

A particular aspect provided herein is the use of an ammonium silane or composition described herein for the treatment of atopic dermatitis. Atopic dermatitis (AD) also known as eczema, is the most common allergic skin disease in the general population. It is a chronic inflammatory skin disease complicated by recurrent bacterial and viral infections that, when left untreated, can lead to significant complications. These infections include Staphylococcus aureus (S. aureus) skin infections, eczema herpeticum, eczema vaccinatum, and eczema coxsackium. More recent studies suggest that skin microbiome including Staphylococcus epidermidis or other coagulase-negative staphylococci may play an important role in controlling S. aureus skin infections in AD. Other studies also suggest that genetic variants in the innate immune response may predispose AD patients to increased risk of viral skin infections. (Ong P Y et al., “Bacterial and Viral Infections in Atopic Dermatitis: a Comprehensive Review.”, Clin Rev Allergy Immunol., 2016, 51(3), 329-337; DOI: 10.1007/s12016-016-8548-5; InformedHealth.org [Internet]. Cologne, Germany: Institute for Quality and Efficiency in Health Care (IQWiG); 2006-. Eczema: Overview. 2013 Sep 26 [Updated 2017 Feb 23]. Available from: https://www.ncbi.nlm.nih.gov/books/NBK279399/).

In certain embodiments, treatment of eczema comprises placing the topical composition containing one or more ammonium silanes described herein or a composition described herein at the site of infection. The length of time that the ammonium silane, or composition is applied is such that antimicrobial treatment is still effective or the infection has resolved. The treatment may be applied continuously, with concurrent successive applications after an appropriate time frame, or in alternation with another treatment for the infection after an appropriate time frame. The ammonium silanes described herein may be applied at the site of infection in a host at the appropriate interval as determined by a healthcare provider.

In some embodiments, the ammonium silanes described herein are placed at the site of infection for a day or less. In other embodiments, the ammonium silanes described herein are placed at a site of infection for a week or more.

In certain embodiments, the method of treatment can be administered once a day (q.d.), twice a day (b.i.d.), three times a day (t.i.d.), four times a day (q.i.d.), once every other day (Q2d), once every third day (Q3d), as needed, or using any dosing schedule that provides treatment of an infection as described herein.

In certain embodiments, a topical formulation is also provided for the treatment of eczema comprising administering an effective amount of a compound described herein, or its pharmaceutically acceptable salt and a topically acceptable carrier.

In certain embodiments, an effective amount of a compound as described herein, or the compound described herein in combination or alternation with, or preceded by, concomitant with or followed by another active agent, can be used in an amount sufficient to (a) inhibit the progression of eczema; (b) cause a regression of eczema; (c) cause a cure of eczema; or inhibit or prevent the development of eczema. Accordingly, an effective amount of a compound or its salt or composition described herein will provide a sufficient amount of the agent when administered to a human to provide a desired benefit.

The treatment period is ideally sufficient time for the active compound to reduce or eliminate the appearance of eczema on the target portion of skin. The treatment period may last for at least 1 week, about two weeks, about 4 weeks, about 8 weeks, or about 12 weeks. The treatment period may extend over multiple months (about 3-12 months) or multiple years. The step of applying the compound or its salt or composition may be accomplished by localized application, i.e., by applying to the targeted area while minimizing delivery to skin surfaces where treatment is not desired, or by applying more generally or broadly to one or more skin surfaces.

Persistent mating-induced endometritis (PMIE)

A particular aspect provided herein is the use of an ammonium silane or composition described herein for the treatment of persistent mating-induced endometritis. Persistent mating-induced endometritis (PMIE) is an inflammatory condition caused by biofilm-induced infection of the endometrium in mares, referred colloquially as ‘dirty’ mares. Endometritis is one of the major causes of mare infertility and breeding failure (Liu et al. (2008). The diagnosis and treatment of endometritis in the mare: yesterday and today. 70(3):415-20). The organisms isolated from mares with PMIE are most often bacteria such as S. zooepidemicus (0-hemolytic), E. coli (haemolytica), P. aeruginosa, Actinomyces pyogenes, Proteus spp., Staphylococcus spp., and Citrobacter spp. and K. pneumoniae, and fungi, which include Candida spp. and Aspergillus spp.

In certain embodiments, the endometritis is persistent mating-induced endometritis (PMIE). In certain embodiments, the PMIE is bacterial PMIE. In certain embodiments, the PMIE is fungal PMIE.

In certain embodiments, an ammonium silane or composition described herein may be used in an aqueous wash solution having anti-microbial effects that help alleviate the symptoms of endometritis and treats the underlying overgrowth of bacterial and/or fungi that cause endometritis. In certain embodiments, the compounds provided herein may be applied in an aqueous wash formulation in any amount that achieves the desired effect. In certain non-limiting examples, the weight percentage of the ammonium silane in the aqueous wash formulation is from about 0.1% to about 25%, or from about 0.1% to about 20%, or about 0.5% to about 10%, or from about 1.0, 2.0, 4.0 or 5.0% to about 10%, or between about 0.5% to about 2.0%. Examples include at least about 0.5, 1.0, 2.0, 3.0, 4.0, 5.0, 10 or 15% by weight. In certain embodiments, the weight percentage of the ammonium silane in the aqueous wash formulation is about 1.0%. In certain embodiments, the pharmaceutical carrier comprises sodium lactate. In certain embodiments, the wash solution is diluted with water prior to administration. In certain embodiments, the wash solution is administered at standard amounts of between about 250 mL to about 500 mL. In certain non-limiting examples, the aqueous wash formulation is administered at an amount of between about 300 mL to about 450 mL, from about 300 mL to about 400 mL, or from about 250 mL, 275 mL, 300 mL to about 350 mL, or from about 500 mL, 450 mL, or 400 mL. The length of time that the ammonium silane, or composition is applied is such that antimicrobial treatment is still effective or the infection has resolved. In certain embodiments, the mare passively expels the aqueous wash composition.

The treatment may be applied continuously, with concurrent successive applications after an appropriate time frame, or in alternation with another treatment for the infection after an appropriate time frame. In certain embodiments, the time frame is 24 hours. In certain embodiments, the antimicrobial treatment is administered on consecutive days. In certain embodiments, the antimicrobial treatment is administered over 3 consecutive days, wherein the antimicrobial treatment is administered about 24 hours apart. The ammonium silanes described herein may be applied at the site of infection in a host at the appropriate interval as determined by a healthcare provider. In certain embodiments, the antimicrobial treatment is administered after estrus. In some embodiments, the antimicrobial treatment is administered using an application device. In certain embodiments, the application device comprises a balloon-tipped catheter. In certain embodiments, the mare is inseminated between about 14 days to 19 days following the final administration of the antimicrobial treatment. In certain embodiments, the mare is inseminated between about 15 days to 17 days following the final administration of the antimicrobial treatment. In certain embodiments, the mare is inseminated between about 15 days following the final administration of the antimicrobial treatment. In certain embodiments, the mare is inseminated between about 16 days following the final administration of the antimicrobial treatment. In certain embodiments, the mare is inseminated between about 17 days following the final administration of the antimicrobial treatment. In certain embodiments, the treatment is administered in cycles. In certain embodiments, the cycles are repeatedly administered after months or years apart. In certain embodiments, the cycles are administered according to the estrus cycle of the subject.

In certain embodiments, the method of treatment can be administered once a day (q.d.), twice a day (b.i.d.), three times a day (t.i.d.), four times a day (q.i.d.), once every other day (Q2d), once every third day (Q3d), as needed, or using any dosing schedule that provides treatment of an infection as described herein. In certain embodiments, the method of treatment can be repeatedly administered less than 24 hours apart.

### Use as a Preservative

In some embodiments, an ammonium silane or composition described herein-is used as a preservative to help slow or prevent the growth of harmful microorganisms in a wide range of products. Accordingly, an ammonium silane or composition described herein may be added to, for example: food to fight spoilage and slow and prevent changes in color, flavor or texture and delay racidity; to medicine and pharmaceuticals to prevent contamination by bacteria and fungi, which may cause disease or infection; cosmetics and personal care products, which include for example: skin moisturizers, perfumes, lipsticks, fingernail polishes, eye and facial makeup preparations, shampoos, permanent waves, hair colors, toothpastes, mouthwashes, and deodorants; to wood products; pharmaceuticals; paints; inks and other products susceptible to contamination.

### Use as a Disinfectant

In some embodiments, an ammonium silane or composition described herein can be used as a disinfectant that repels water and dirt. Examples of appropriate use include, but are not limited to, the reduction of hospital acquired infections, such as surgical site disinfection, disinfection of instruments, and prevention of microbial colonization of humidifiers and breathing machines; and, hard-surface cleaning and deodorization which are also used and useful in commercial and residential settings.

### Powder Formulations for Use in the Present Invention

In some embodiments, one or more active ammonium silanes described herein can be provided as a powder formulation. Powder formulations can be prepared by removing any residual solvents, for example by evaporation or, in some cases, sublimation away from residual solvent and impurities. In some embodiments, lyophilization is used to create the powder for the formulation as it typically maintains the integrity of the product due to the low temperature used in processing. Additionally, lyophilized solids can be reconstituted much more quickly and easily due to the presence of microscopic pores formed by the process. The high vacuum used during lyophilization ensures thorough removal of any undesired volatile components such as methanol, ethanol, or other volatile organic substances. In some embodiments, the powder formulation of the ammonium silanes and products described herein contains less than about 5%, about 4%, about 3%, about 2%, about 1%, about 0.5%, or about 0.01% of methanol by weight. Methods for the lyophilization of solids, particularly of sensitive materials used in pharmaceutical applications, are known in the art. Lyophilization may be performed using any number of commercially available apparatuses, for example a shelf-cabinet, contact, radiant, or microwave assisted lyophilizer.

Typical lyophilization procedures are composed of four steps. In the first step (Pre-Treatment), the active ammonium silane is dissolved in an appropriate solvent and additional excipients are optionally added as required to increase stability, preserve appearance, or improve later processing. Additionally, solutions of the active ammonium silane may be concentrated as appropriate to aid in the freezing and later sublimation processes. Additionally, components may undergo initial individual quick freezing to ensure formation of a free-flowing solid upon completion of the lyophilization.

In the second step (Freezing), the solution of the active ammonium silane is frozen in a vessel below its triple point to ensure that sublimation rather than melting will occur. Optionally, the material can be cycled up and down in temperature in a process called annealing. If the ammonium silane to be lyophilized is an amorphous solid, it may not have a triple point and instead has a critical point. Amorphous solids must be maintained below the critical point temperature during the entirety of the lyophilization process to prevent melt-back or collapse of the solid during the subsequent drying steps. For sensitive materials, the freezing step is often performed quickly by lowering the temperature of the material to between about −50 and −80° C. This prevents the formation of large solvent crystals that may diminish the structure integrity of the material being lyophilized and lead to poor texture.

In the third step (Primary Drying), the pressure of the vessel is lowered (typically to the range of a few millibars) and a minimum of heat is applied to the material for the solvent to sublime. Pressure is typically controlled by the application of a partial vacuum. A small amount of heat may be applied to facilitate sublimation of the solvent molecules. Typically, this heat is applied via conduction or radiation due to the low air density within the vessel.

In the final step (Secondary Drying), the temperature is raised higher than in the primary drying phase to remove any residual unfrozen solvent molecules. The rise in temperature is required to break any physico-chemical interactions that may have formed between the solvent molecules and the frozen material. Additionally, the pressure is typically lowered compared to the primary drying step to encourage desorption.

Upon completion of the lyophilization process, the vacuum is typically broken with an inert gas, for example nitrogen, and sealed in an appropriate container. Typical containers include sealed ampoules comprising sealed glass that is broken open at the time of desired application. The active material may be subsequently reconstituted at the time of application by using an appropriate carrier such as those that are described herein, for example sterile water or glycerin.

### Antimicrobial Compositions

Active ammonium silanes described herein can be administered to a host in need thereof as the neat chemical but are more typically administered as antimicrobial composition that includes an effective amount for a host, typically a human, in need of such treatment of an active ammonium silane as described herein or combinations thereof.

In some embodiments, the disclosure provides antimicrobial compositions comprising an effective amount of an ammonium silane together with at least one pharmaceutically acceptable carrier for any of the uses described herein. The pharmaceutical composition may contain an ammonium silane as the only active agent.

In an alternative embodiment, the pharmaceutical composition comprises an ammonium silane and at least one additional active agent. In a typical formulation, an ammonium silane described herein is provided in a sterilized, powder, or solid form that is reconstituted on use.

An effective amount of an active ammonium silane as described herein, or the active ammonium silane described herein in combination or alternation with, or preceded by, concomitant with or followed by another active agent, can be used in an amount sufficient to (a) inhibit the progression of an infection described herein; (b) cause a regression of an infection described herein; (c) cause a cure of an infection described herein; or inhibit or prevent the development of an infection described herein. Accordingly, an effective amount of an active ammonium silane, or composition described herein will provide a sufficient amount of the active agent when administered to a patient to provide a clinical benefit.

The exact amount of the active ammonium silane or antimicrobial composition described herein to be delivered to the host, typically a human, in need thereof, will be determined by the health care provider to achieve the desired clinical benefit.

In certain embodiments the antimicrobial composition is in a dosage form that contains from about 0.1 mg to about 2000 mg, from about 10 mg to about 1000 mg, from about 100 mg to about 800 mg, or from about 200 mg to about 600 mg of the active ammonium silane and optionally from about 0.1 mg to about 2000 mg, from about 10 mg to about 1000 mg, from about 100 mg to about 800 mg, or from about 200 mg to about 600 mg of an additional active agent in a unit dosage form.

Examples are dosage forms with at least about 0.5, 1, 1.5, 2, 2.5, 3, 3.5, 4, 4.5, 5, 10, 15, 20, 25, 50, 75, 100, 150, 200, 250, 300, 350, 400, 450, 500, 550, 600, 650, 700, 750, 800, 900, 1000, 1100, 1200, 1250, 1300, 1400, 1500, or 1600 mg of active ammonium silane. In some embodiments, the dosage form has at least about 1 mg, 5 mg, 10 mg, 25 mg, 50 mg, 75 mg, 100 mg, 200 mg, 400 mg, 500 mg, 600 mg, 1000 mg, 1200 mg, or 1600 mg of active ammonium silane. The dosage form can be administered, for example, once a day (q.d.), twice a day (b.i.d.), three times a day (t.i.d.), four times a day (q.i.d.), once every other day (Q2d), once every third day (Q3d), as needed, or any dosage schedule that provides treatment of a disorder described herein.

The antimicrobial composition may for example include a molar ratio of the active ammonium silane and an additional active agent that achieves the desired result. For example, the pharmaceutical composition may contain a molar ratio of at least about 0.5:1, at least about 1:1, at least about 2:1, at least about 3:1, or from about 1.5:1 to about 4:1 of an additional active agent in combination with the active ammonium silane (additional active agent: active compound) described herein.

Ammonium silanes disclosed herein or used as described herein may be administered topically, by powder, spray, cream, gel, putty, ointment, foam, suppository, via implant, including ocular implant, coatings, epoxy, transdermally, bone graft compositions, dermatological formulation, or as an ophthalmic solution, in dosage unit formulations containing conventional pharmaceutically acceptable carriers. For ocular delivery, the ammonium silane can be administered, as desired, for example, as a solution, suspension, or other formulation via an immediate or controlled release fashion or via an ocular device, or topically administered formulation, for example a solution or suspension provided as an eye drop.

The antimicrobial composition may be formulated as any pharmaceutically useful form, e.g., as an aerosol, a cream, a gel, ointment, foam, a microparticle, a nanoparticle, an injection or infusion solution, a transdermal patch, a subcutaneous patch, a suppository, a dry powder, in or on a medical device, parenteral formulation, dermatological formulation, or an ophthalmic solution or suspension. Some dosage forms are subdivided into suitably sized unit doses containing appropriate quantities of the active components, e.g., an effective amount to achieve the desired purpose.

Compositions, and methods of manufacturing such compositions, suitable for administration as contemplated herein are known in the art. Examples of known techniques include, for example, U.S. Pat. Nos. 5,723,269, 9,060,938, and U.S, published Patent Application 20220016312, incorporated by reference herein.

The antimicrobial compositions contemplated here can optionally include a carrier. Carriers must be of sufficiently high purity and sufficiently low toxicity to render them suitable for administration to the patient being treated. The carrier can be inert or it can possess pharmaceutical benefits of its own, for example, as bone scaffolding or wound moisturizing. The amount of carrier employed in conjunction with the ammonium silane is sufficient to provide a practical quantity of material for administration per unit dose of the compound.

Classes of carriers include, but are not limited to binders, buffering agents, coloring agents, diluents, disintegrants, emulsifiers, fillers, flavorants, glidents, lubricants, pH modifiers, preservatives, putty, stabilizers, surfactants, solubilizers, tableting agents, and wetting agents.

Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Examples of other matrix materials, fillers, or diluents include lactose, mannitol, xylitol, microcrystalline cellulose, calcium diphosphate, and starch. Examples of surface-active agents include sodium lauryl sulfate and polysorbate 80.

Examples of drug complexing agents or solubilizers include polyethylene glycols, caffeine, xanthene, gentisic acid and cylodextrins.

Examples of disintegrants include sodium starch glycolate, sodium alginate, carboxymethyl cellulose sodium, methyl cellulose, colloidal silicon dioxide, and croscarmellose sodium. Examples of binders include methyl cellulose, microcrystalline cellulose, starch, and gums such as guar gum, and tragacanth.

Examples of lubricants include magnesium stearate and calcium stearate.

Examples of pH modifiers include acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids. Optional other active agents may be included in a pharmaceutical composition, which do not substantially interfere with the activity of an ammonium silane described herein.

Non-limiting examples of aqueous solutions as can be used in the carrier include distilled water, saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, ProVisc® diluted with PBS, Krebs buffer, Dulbecco's PBS, normal PBS, sodium hyaluronate solution, simulated body fluids, plasma platelet concentrate, tissue culture medium, an aqueous solution comprising an organic solvent, and mixtures thereof. The solution in all instances can be sterilized in a suitable manner known to those in the art.

In certain examples the antimicrobial composition contains a polymeric material. The polymeric material should be biocompatible such that it can be administered to a patient without an undesired effect. Polymers are well known in the art and are the subject of extensive literature and patents. In certain embodiments, the polymer is present in an amount effective to provide the desired viscosity and moistening properties as needed for the desired application, for example in the treatment of infected wounds. The specific amount of polymer used depends on a number of factors including, for example and without limitation, the specific chemical composition of the polymer used, the molecular weight of the specific polymer used, the viscosity of the desired antimicrobial composition, and the level of water retainment and release desired for the particular polymer.

In certain embodiments, the antimicrobial composition is used in polymeric material for medical, personal or industrial applications. Examples for medical applications include, but are not limited to, wound care materials, medical devices, proper protective equipments, artificial cartilage, bone grafts, biomaterials, catheters, other implantable devices and non-implant devices (such as surgical sponges, and packaging), medical applications or devices, for example orthopedic or dental applications or devices, ophthalmic solutions or suspensions, short-term wound packing, direct application to eye tissues, hydrophilic coatings for catheters, leads, or vascular embolic agents, and the like.

In certain embodiments, the antimicrobial composition is used in polymeric material for coatings, adhesives, and sealants for medical devices, including pacemakers, contact lenses, dentures, prosthetic devices, heart valves and joints; biomaterials, implantable devices, non-implant devices, wound care material, personal protective material such as gloves, face masks, surgical/hospital gowns, cloth material, sheets, woven or nonwoven materials, sponges; medical devices such as orthopedic or dental devices, ophthalmic solutions or suspensions, ocular inserts, ocular film, targeted ocular delivery, short-term wound packing, direct application to eye tissues, hydrophilic coatings for catheters, leads etc., or vascular embolic agents, and the like.

In certain embodiments the polymeric material comprises polymers thermoplastic polymer, thermosetting polymer, biodegradable polymer, bioresorbable polymer, modified polymers, crosslinked polymers, polymers for controlled delivery, hydrogels, hydrocolloids, liquid forming polymer, gel forming polymer, silicone-based polymeric material, film forming polymer, adhesive polymer, polymers for controlled delivery copolymers, polymers for medical uses, copolymers, or mixtures thereof, and the like.

In certain embodiments, the silane ammonium silane described herein and the polymeric material is in a ratio of at least 1:1000:1000:1; 1:500:500:1, 1:300:300:1, 1:250:250:1; 1:200:200:1; 1:150:150:1; 1:100:100:1; 1:75:75:1; 1:50:50:1; 1:40:40:1; 1:30:30:1, 1:25:25:1; 1:20:20:1; 2:25:15:1; 1:10:10:1; 1:5:5:1; 1:3:3:1; 1:2:2:1; or 1:1.

In certain embodiments, the composition of the silane ammonium silane and polymer material forms a solid, stable composition.

In an additional embodiment, a dry powder formulation containing an ammonium silane or composition described herein is applied under, to and around an implant after installation/insertion and subsequently wetted upon interaction with bodily fluids at the surgical site.

In certain embodiments, the composition forms a clear stable solution. In certain embodiments, the composition is stable for at least one month. In certain embodiments, the composition is stable for at least 2 months. In certain embodiments, the composition is stable for at least 3 months.

In certain embodiments, the composition comprises a wash solution. In certain embodiments, the pharmaceutical carrier comprises sodium lactate. In certain embodiments, the wash solution is diluted with water prior to administration. In certain embodiments, the wash solution is administered at standard amounts of between about 250 mL to about 500 mL. In certain embodiments, the wash solution is administered using a balloon-tipped catheter or under a defined pressure using a syringe.

In certain embodiments, a silane ammonium silane described herein is combined with a polymer described herein to form flexible film. In certain embodiments, the film exists with varying thickness level suitable for a particular application described herein.

In certain embodiments, a silane ammonium silane described herein is incorporated into polyvinyl alcohol. In certain embodiments, the resultant product is used for medical applications and devices. For example, soft contact lenses, eye drops, embolization particles, tissue adhesion barriers, and as artificial cartilage and meniscus. In certain embodiments, the resultant product is a medical device implant material for cartilage replacement. In certain embodiments, the resultant product is used for transient to short-term wound packing, direct application to eye tissues, hydrophilic coatings for catheters, leads, or vascular embolic agents. See, for example, Baker, M., et al., “A review of polyvinyl alcohol and it uses in cartilage and orthopedic applications”, Wiley Online Library. DOI: 10.1002/jbm.b.32694 (2012), incorporated herein.

In certain embodiments, the polymer is a polyvinyl alcohol hydrogel. In certain embodiments, the polyvinyl alcohol hydrogen is useful for drug delivery systems. In certain embodiments, the drug delivery system comprises ocular inserts, ocular films, nanoparticles, microspheres, floating microspheres, microchannels, mucoadhesive or targeted drug delivery, and the like. In certain embodiments, the drug delivery system is ocular inserts, ocular films, microspheres, floating microspheres, or targeted drug delivery.

Polyvinyl alcohol hydrogels are biocompatible and toxicologically safe polymer used as a matrix for sustained release hydrogel drug delivery systems in solid, liquid and semi-solid formations. Polyvinyl alcohol hydrogels have excellent physical properties like mucoadhesive, swelling make it suitable for the diverse drug delivery applications.” See, e.g., Gajra, Balaram & Pandya et al., “Poly vinyl alcohol Hydrogel and its Pharmaceutical and Biomedical Applications: A Review”, International Journal of Pharmaceutical Research, 2011, 4. 20-26, incorporated herein by reference.

In some examples, the antimicrobial composition contains a hydrogel. The hydrogel should be biocompatible such that it can be administered to a patient without an undesired effect. Hydrogels are well known in the art and are the subject of extensive literature and patents. The hydrogel is present in an amount effective to provide the desired viscosity and moistening properties as needed for the desired application, for example in the treatment of infected wounds. The specific amount of hydrogel used depends on a number of factors including, for example and without limitation, the specific chemical composition of hydrogel used, the molecular weight of the specific hydrogel used, the viscosity of the desired antimicrobial composition, and the level of water retainment and release desired for the particular hydrogel.

In some embodiments, the hydrogel controls the rate of release of one or more ammonium silanes described herein. In some embodiments, the hydrogel is biodegradable. Examples of useful hydrogel carriers include, but are not limited to, poly(vinyl alcohol), sodium polyacrylate, poly(acrylamide), poly(N-vinyl-2-pyrrolidone), poly(N-isopropylacrylamide), cross-linked carboxymethylcellulose, cross-linked polyethylene glycol, poly(lactic acid), hyaluronic acid, sodium alginate, agarose, starch, chitosan, methylcellulose, polyethylene oxide, amorphous hydrogel, crosslinked polymer gels with high water content, copolymers thereof, derivatives thereof, mixtures thereof, and the like.

In some embodiments, the antimicrobial composition contains a hydrocolloid. In some embodiments, the hydrocolloid may interact with the site of infection by forming a gel. A hydrocolloid may be present to provide combined moisture and absorptivity in sites where it is deemed necessary. The hydrocolloid may include, but is not limited to, natural gums such as Arabic gum, ghatti gum, karaya gum, tragacanth gum, guar gum, locust bean gum, acacia gum; seaweed extracts such as agar, algin, alginate salts and carrageenan, cereal gums, starches, microbial gums such as dextran gum and xanthan gum, pectins, gelatins, casein, collagens, polyvinylpyrrolidone, low methoxyl pectin, propyleneglycol alginates, carboxymethyl locust bean gum, carboxymethyl guar gum, and modified forms that have been oxidized, acetylated, carboxylated, esterified, methylated, aminated, etherated, sulfated, borated, or phosphated, absorbant colloidal material with elastomers covered with polyurethane; and the like.

In certain embodiments, the antimicrobial composition for administration includes an ammonium silane as described herein and optionally comprises one or more of a chitosan, phosphoglyceride; phosphatidylcholine; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol such as polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid; pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan, mannitol, sorbitol, xylitol, erythritol, maltitol, and lactitol, a pluronic polymer, polyethylene, polycarbonate (e.g. poly(1,3-dioxan-2one)), polyanhydride (e.g. poly(sebacic anhydride)), polypropylfumerate, polyamide (e.g. polycaprolactam), polyacetal, polyether, polyester (e.g., polylactide, polyglycolide, polylactide-co-glycolide, polycaprolactone, polyhydroxyacid (e.g. poly((P-hydroxyalkanoate))), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-α-Tocopheryl polyethylene glycol 1000 succinate), gelatin, titanium dioxide, polyvinylpyrrolidone (PVP), hydroxypropyl methyl cellulose (HPMC), hydroxypropyl cellulose (HPC), methyl cellulose (MC), block copolymers of ethylene oxide and propylene oxide (PEO/PPO), polyethyleneglycol (PEG), sodium carboxymethylcellulose (NaCMC), hydroxypropylmethyl cellulose acetate succinate (HPMCAS).

In some embodiments, the antimicrobial composition may include polymers for controlled delivery of the described compounds, including, but not limited to pluronic polymers, polyesters (e.g., polylactic acid, poly(lactic-co-glycolic acid, polyethylene terephthalate (PET), glycol-modified polyethylene terephthalate (PETG), polybutylene terephthalate (PBT), polycyclohexylenedimethylene terephthalate (PCT), polycyclohexylendimethylene terephthalate glycol (PCTG), acid-modified polycyclohexylenedimethylene terephthalate (PCTA), polytrimethylene terephthalate (PTT), woven and non-woven polyethylene terephthalate (PET), spun-bonded, spun-laced, embossed polyethylene terephthalate, LDPE non-woven polyethylene terephthalate, glycol-modified polyethylene terephthalate (PETG), or another kind of polyester which can include monomers or co-monomers that have other carboxylic acid or alcohol functions), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers may be modified with polyethylene glycol (PEG), with a carbohydrate, and/or with acyclic polyacetals derived from polysaccharides. See, e.g., Papisov, 2001, ACS Symposium Series, 786:301, incorporated by reference herein.

In certain embodiments, additional polymers include but are not limited to, polyolefin (including cyclic polyolefin), including polypropylene and polyethylene; polyvinyl chloride; polystyrenes; polyvinylidene chlorides; polynorbornene; polyimide; polyamide; polyurethane; polystyrene; polyvinylidene chloride; polyvinyl chloride; polylactic acid; or a combination or combinations thereof.

In some embodiments, the antimicrobial composition contains a bio-degradable bone filler. Bone fillers are bio-degradable materials such as granules of dried human bone, and commonly used in dentistry and orthopedics to fill in bone cavities.

In some embodiments, the antimicrobial composition contains a growth promoter, pain control agent, chemotherapeutic agent, or an anti-inflammatory agent. Growth promoters are known in the art and include, but are not limited to Platelet Derived Growth Factor (PDGF), including PDGF-BB, insulin-like growth factor (IGF), transforming growth factor-beta 1 (TGF-β1), bone morphogenetic protein (BMP), including BMP-2 and BMP-7, epidermal growth factor (EGF), fibroblast growth factor, for example FGF-2 and FGF-7, hepatocyte growth factor (HGF), vascular endothelial growth factor (VEGF), including for example VEGF-A. Pain control agents are known in the art and include, but are not limited to, menthol, methyl salicylate (oil of evergreen), camphor, salicylates, acetaminophen, non-steroidal antiflammatory drug including, for example, aspirin, ibuprofen, naproxen, nabumetone, a COX-2 inhibitor, for example, celecoxib, rofecoxib, valdecoxib, capsaicin, and lidocaine. Chemotherapeutic agents are well known in the art and include, but are not limited to, an alkylating agent, a DNA intercalator, a protein synthesis inhibitor, an inhibitor of DNA or RNA synthesis, a DNA base analog, a topoisomerase inhibitor, a telomerase inhibitor, a telomeric DNA binding compound, and a combination thereof. Anti-inflammatories are well known in the art and include, but are not limited to, NSAIDs, immune selective anti-inflammatory derivatives (ImSAIDs), anitleukotrienes, and endocannabinoids.

In some embodiments, the antimicrobial composition contains a biodegradable polymer. The biodegradable polymer should be biocompatible such that it can be administered to a patient without an undesired effect. Biodegradable polymers are well known in the art and are the subject of extensive literature and patents. The biodegradable polymers or combination of polymers can be selected to provide the desired characteristics for the chosen application including, but not limited to, the appropriate mix of hydrophobic and hydrophilic qualities, half-life and degradation kinetics, compatibility with one or more ammonium silanes described herein to be delivered, and the appropriate behavior at the site of application.

In some embodiments, the biodegradable polymer gelates in the presence of an aqueous solution such as is present in the site of a wound or infection.

In some embodiments, the biodegradable polymer carrier provides release of one or more ammonium silanes described herein into the site of infection at a desired rate. Examples of useful biodegradable polymers include, but are not limited to, poly(lactic acid), polyglycolic acid, poly(D,L-lactide-co-glycolide), poly(D,L-lactic acid), polyesters, poly(caprolactone), poly(3-hydroxybutyrate), poly(s-caproic acid), poly(p-dioxanone), poly(propylene fumarate), poly(orther esters), polyol/diketene acetals, poly(sebacic anhydride), poly(maleic anhydride), poly(carboxybis-carboxyphenoxyphosphazene), poly[bis(p-carboxyphenoxy)methane], poly(amino acids), or copolymers thereof.

Optional active ingredients may be included in the antimicrobial composition which do not substantially interfere with the activity of one or more ammonium silanes described herein used in the present invention. In certain embodiments, two or more of the carrier components may be combined as deemed necessary for the particular application.

In some embodiments, the antimicrobial composition further comprises one or more additional additives. These ammonium silanes may be included to increase the efficacy of the desired antimicrobial composition in penetrating the site of infection being treated, to aid in tissue healing or symptom abatement at the site of infection if it is deemed necessary, or to increase the effective shelf life of the antimicrobial composition either alone or in combination with other active agents.

In some embodiments, the antimicrobial composition further comprises a surfactant. The surfactant can be added to help facilitate penetration of one or more ammonium silanes described herein into subsurface layers of a biofilm present at the site of infection by disrupting the complex hydrophobic/hydrophilic interactions between biofilm layers if one or more ammonium silanes alone prove insufficient for this purpose. The surfactant additive selected can be chosen to provide desired characteristics to the antimicrobial composition, such as stability of the surfactant and one or more ammonium silanes in the appropriate carrier, level of desired penetration into the biofilm, and level of reactivity with other components in the composition. An appropriate surfactant would be able to be chosen by one skilled in the art. In some embodiments, the surfactant can facilitate leaching of one or more ammonium silanes described herein from the selected carrier.

In some embodiments, the surfactant can facilitate leaching of one or more ammonium silanes described herein from either a formulated microparticle or polymeric nanoparticle. Examples of appropriate surfactants include, but are not limited to, octenidine dihydrochloride, cetrimonium bromide (CTAB), cetylpyridinium chloride (CPC), benzalkonium chloride (BAC), benzethonium chloride (BZT), dimethyldioctadecylammonium chloride, dioctadecyldimethylammonium bromide (DODAB), cocamidopropyl hydroxysultaine (CAHS), cocamidopropyl betaine (CAPB), cocamide MEA, sodium oxychlorosene, and combinations thereof.

In some embodiments, the antimicrobial composition further comprises a buffer. Some of the other potential additives to the antimicrobial composition may require very narrow pH ranges to optimally function. A buffer can be provided at appropriate concentrations to maintain an optimized pH range. The optimized buffer for the particularly desired application would be known to known to those skilled in the art. Examples of appropriate buffers include, but are not limited to, salts of citrates, sulfonates, carbonates, acetate, borates, gluconates, phosphates, or combinations thereof.

In some embodiments, the antimicrobial composition further comprises appropriate enzymes. The enzymes can be added to assist in disrupting the established biofilm by either decomposition of the extracellular polymeric substances (EPS) or by suppressing cell to cell communications, sent via ion channels in the form of electrical signals, to coordinate their behavior. In some embodiments, the enzymes may be proteolytic enzymes. Proteolytic enzymes may be able to act upon some of the polymeric materials present in the EPS, allowing increased penetration of the antimicrobial composition. Examples of proteolytic enzymes include, but are not limited to, collagenase, cellulase, keratinase, papain, bromelain, trypsin, thermolysin, and combinations thereof.

In some embodiments, the antimicrobial composition further comprises an appropriate tissue or bone growth promoter. In some applications, the biofilm-induced infection that is being treated is present within a wound. Inclusion of an appropriate tissue growth promoter may help facilitate regrowth of tissue within the present wound during the time of infection treatment with the dressing. Examples of appropriate tissue growth promoters include, but are not limited to, endothelial cell growth factors (ECGF), epidermal growth factors (EGF), fibroblast growth factors (FGF), hepatocyte growth factors (HGF), nerve growth factors (NGF), platelet-derived growth factors (PDGF), transforming growth factors (TGF), or combinations thereof. Examples of bone growth promoters include, but are not limited to, Teripartatide, Abaloparatide, and, Romosozumab.

In some embodiments, the antimicrobial composition further comprises a preservative. While one or more ammonium silanes described herein antimicrobial in nature, an additional preservative may be optionally included dependent on the desired shelf life of the antimicrobial composition. Examples of appropriate preservatives include, but are not limited to, methylparaben, propylparaben, benzyl alcohol, benzalkonium chloride, sorbic acid, phenol, phenylethyl alcohol, BHA, BHT, or combinations thereof.

In some embodiments, the antimicrobial composition further comprises an antioxidant. An antioxidant may be necessary to stabilize any other additive present within the antimicrobial composition from air oxidation over a suitable shelf life. Examples of appropriate antioxidants include, but are not limited to ascorbic acid, BHA, BHT, sodium bisulfite, vitamin E, sodium metabisulfite, propyl gallate, or combinations thereof.

In some embodiments, the antimicrobial composition further comprises an astringent. Addition of an astringent to the antimicrobial composition may be desirable by causing surface tissues that contain an infection to shrink, allowing ready penetration of the antimicrobial composition into the infected space. Examples of appropriate astringents include, but are not limited to, zinc oxide, ferric oxide, zinc sulfate, silver nitrate, potassium permanganate, aluminum chloride, aluminum acetate, formaldehyde, Burow's solution, tincture of benzoin, or combinations thereof.

Antimicrobial compositions suitable for topical application to the skin preferably take the form of an ointment, cream, lotion, foam, paste, gel, spray, aerosol, or oil. Carriers which may be used include petroleum jelly, lanoline, polyethylene glycols, alcohols, transdermal enhancers, and combinations of two or more thereof.

Antimicrobial compositions suitable for transdermal administration may be presented as discrete patches adapted to remain in intimate contact with the epidermis of the recipient for a prolonged period of time. Antimicrobial compositions suitable for transdermal administration may also be delivered by iontophoresis (see, for example, Pharmaceutical Research 3 (6):318 (1986)) and typically take the form of an optionally buffered aqueous solution of the active ammonium silane. In some embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Many methods and devices for drug delivery to the eye are known in the art. Non-limiting examples are described in the following patents and patent applications (fully incorporated herein by reference). Examples are U.S. Pat. No. 8,192,408 titled “Ocular trocar assembly” (Psivida Us, Inc.); U.S. Pat. No. 7,585,517 titled “Transcleral delivery” (Macusight, Inc.); U.S. Pat. Nos. 5,710,182 and 5,795,913 titled “Ophthalmic composition” (Santen OY); U.S. Pat. No. 8,663,639 titled “Formulations for treating ocular diseases and conditions”, U.S. Pat. No. 8,486,960 titled “Formulations and methods for vascular permeability-related diseases or conditions”, U.S. Pat. Nos. 8,367,097 and 8,927,005 titled “Liquid formulations for treatment of diseases or conditions”, U.S. Pat. No. 7,455,855 titled “Delivering substance and drug delivery system using the same” (Santen Pharmaceutical Co., Ltd.); WO/2011/050365 titled “Conformable Therapeutic Shield For Vision and Pain” and WO/2009/145842 titled “Therapeutic Device for Pain Management and Vision” (Forsight Labs, LLC); U.S. Pat. Nos. 9,066,779 and 8,623,395 titled “Implantable therapeutic device”, WO/2014/160884 titled “Ophthalmic Implant for Delivering Therapeutic Substances”, U.S. Pat. Nos. 8,399,006, 8,277,830, 8,795,712, 8,808,727, 8,298,578, and WO/2010/088548 titled “Posterior segment drug delivery”, WO/2014/152959 and US20140276482 titled “Systems for Sustained Intraocular Delivery of Low Solubility Compounds from a Port Delivery System Implant”, U.S. Pat. Nos. 8,905,963 and 9,033,911 titled “Injector apparatus and method for drug delivery”, WO/2015/057554 titled “Formulations and Methods for Increasing or Reducing Mucus”, U.S. Pat. Nos. 8,715,712 and 8,939,948 titled “Ocular insert apparatus and methods”, WO/2013/116061 titled “Insertion and Removal Methods and Apparatus for Therapeutic Devices”, WO/2014/066775 titled “Ophthalmic System for Sustained Release of Drug to the Eye”, WO/2015/085234 and WO/2012/019176 titled “Implantable Therapeutic Device”, WO/2012/065006 titled “Methods and Apparatus to determine Porous Structures for Drug Delivery”, WO/2010/141729 titled “Anterior Segment Drug Delivery”, WO/2011/050327 titled “Corneal Denervation for Treatment of Ocular Pain”, WO/2013/022801 titled “Small Molecule Delivery with Implantable Therapeutic Device”, WO/2012/019047 titled “Subconjunctival Implant for Posterior Segment Drug Delivery”, WO/2012/068549 titled “Therapeutic Agent Formulations for Implanted Devices”, WO/2012/019139 titled “Combined Delivery Methods and Apparatus”, WO/2013/040426 titled “Ocular Insert Apparatus and Methods”, WO/2012/019136 titled “Injector Apparatus and Method for Drug Delivery”, WO/2013/040247 titled “Fluid Exchange Apparatus and Methods” (ForSight Vision4, Inc.).

Additional non-limiting examples of how to deliver the active ammonium silanes are provided in WO/2015/085251 titled “Intracameral Implant for Treatment of an Ocular Condition” (Envisia Therapeutics, Inc.); WO/2011/008737 titled “Engineered Aerosol Particles, and Associated Methods”, WO/2013/082111 titled “Geometrically Engineered Particles and Methods for Modulating Macrophage or Immune Responses”, WO/2009/132265 titled “Degradable compounds and methods of use thereof, particularly with particle replication in non-wetting templates”, WO/2010/099321 titled “Interventional drug delivery system and associated methods”, WO/2008/100304 titled “Polymer particle composite having high fidelity order, size, and shape particles”, WO/2007/024323 titled “Nanoparticle fabrication methods, systems, and materials” (Liquidia Technologies, Inc. and the University of North Carolina at Chapel Hill); WO/2010/009087 titled “Iontophoretic Delivery of a Controlled-Release Formulation in the Eye”, (Liquidia Technologies, Inc. and Eyegate Pharmaceuticals, Inc.) and WO/2009/132206 titled “Compositions and Methods for Intracellular Delivery and Release of Cargo”, WO/2007/133808 titled “Nano-particles for cosmetic applications”, WO/2007/056561 titled “Medical device, materials, and methods”, WO/2010/065748 titled “Method for producing patterned materials”, WO/2007/081876 titled “Nanostructured surfaces for biomedical/biomaterial applications and processes thereof” (Liquidia Technologies, Inc.).

Additional non-limiting examples of methods and devices for drug delivery to the eye include, for example, WO2011/106702 and U.S. Pat. No. 8,889,193 titled “Sustained delivery of therapeutic agents to an eye compartment”, WO2013/138343 and U.S. Pat. No. 8,962,577 titled “Controlled release formulations for the delivery of HIF-1 inhibitors”, WO/2013/138346 and US2013/0272994 titled “Non-Linear Multiblock Copolymer-Drug Conjugates for the Delivery of Active Agents”, WO2005/072710 and U.S. Pat. No. 8,957,034 titled “Drug and Gene Carrier Particles that Rapidly Move Through Mucus Barriers”, WO2008/030557, US2010/0215580, US2013/0164343 titled “Compositions and Methods for Enhancing Transport Through Mucous”, WO2012/061703, US2012/0121718, and US2013/0236556 titled “Compositions and Methods Relating to Reduced Mucoadhesion”, WO2012/039979 and US2013/0183244 titled “Rapid Diffusion of Large Polymeric Nanoparticles in the Mammalian Brain”, WO2012/109363 and US2013/0323313 titled “Mucus Penetrating Gene Carriers”, WO 2013/090804 and US2014/0329913 titled “Nanoparticles with enhanced mucosal penetration or decreased inflammation”, WO2013/110028 titled “Nanoparticle formulations with enhanced mucosal penetration”, WO2013/166498 and US2015/0086484 titled “Lipid-based drug carriers for rapid penetration through mucus linings” (The Johns Hopkins University); WO2013/166385 titled “Pharmaceutical Nanoparticles Showing Improved Mucosal Transport”, US2013/0323179 titled “Nanocrystals, Compositions, And Methods that Aid Particle Transport in Mucus” (The Johns Hopkins University and Kala Pharmaceuticals, Inc.); WO/2015/066444 titled “Compositions and methods for ophthalmic and/or other applications”, WO/2014/020210 and WO/2013/166408 titled “Pharmaceutical nanoparticles showing improved mucosal transport” (Kala Pharmaceuticals, Inc.); U.S. Pat. No. 9,022,970 titled “Ophthalmic injection device including dosage control device”, WO/2011/153349 titled “Ophthalmic compositions comprising pbo-peo-pbo block copolymers”, WO/2011/140203 titled “Stabilized ophthalmic galactomannan formulations”, WO/2011/068955 titled “Ophthalmic emulsion”, WO/2011/037908 titled “Injectable aqueous ophthalmic composition and method of use therefor”, US2007/0149593 titled “Pharmaceutical Formulation for Delivery of Receptor Tyrosine Kinase Inhibiting (RTKi) Compounds to the Eye”, U.S. Pat. No. 8,632,809 titled “Water insoluble polymer matrix for drug delivery” (Alcon, Inc.).

In another aspect, an ocular formulation is provided comprising one or more ammonium silanes described herein, in a carrier that is suitable to the eye. The appropriate carrier must avoid ammonium silanes that are toxic or irritating to the eye to prevent unwanted side effects or damage to the eye. Examples of components that are not suitable for use in an ocular formulation include corrosives such as strongly alkaline or acidic substances such as urea or ammonia, strong surfactants, and substances with known ocular toxicity such as methanol and hydrogen peroxide.

Additional non-limiting examples of drug delivery devices and methods include, for example, US20050009910 titled “Delivery of an active drug to the posterior part of the eye via subconjunctival or periocular delivery of a prodrug”, US 20130071349 titled “Biodegradable polymers for lowering intraocular pressure”, U.S. Pat. No. 8,481,069 titled “Tyrosine kinase microspheres”, U.S. Pat. No. 8,465,778 titled “Method of making tyrosine kinase microspheres”, U.S. Pat. No. 8,409,607 titled “Sustained release intraocular implants containing tyrosine kinase inhibitors and related methods”, U.S. Pat. No. 8,512,738 and US 2014/0031408 titled “Biodegradable intravitreal tyrosine kinase implants”, US 2014/0294986 titled “Microsphere Drug Delivery System for Sustained Intraocular Release”, U.S. Pat. No. 8,911,768 titled “Methods For Treating Retinopathy With Extended Therapeutic Effect” (Allergan, Inc.); U.S. Pat. No. 6,495,164 titled “Preparation of injectable suspensions having improved injectability” (Alkermes Controlled Therapeutics, Inc.); WO 2014/047439 titled “Biodegradable Microcapsules Containing Filling Material” (Akina, Inc.); WO 2010/132664 titled “Compositions And Methods For Drug Delivery” (Baxter International Inc. Baxter Healthcare SA); US20120052041 titled “Polymeric nanoparticles with enhanced drugloading and methods of use thereof” (The Brigham and Women's Hospital, Inc.); US20140178475, US20140248358, and US20140249158 titled “Therapeutic Nanoparticles Comprising a Therapeutic Agent and Methods of Making and Using Same” (BIND Therapeutics, Inc.); U.S. Pat. No. 5,869,103 titled “Polymer microparticles for drug delivery” (Danbiosyst UK Ltd.); U.S. Pat. No. 8,628,801 titled “Pegylated Nanoparticles” (Universidad de Navarra); US2014/0107025 titled “Ocular drug delivery system” (Jade Therapeutics, LLC); U.S. Pat. No. 6,287,588 titled “Agent delivering system comprised of microparticle and biodegradable gel with an improved releasing profile and methods of use thereof”, U.S. Pat. No. 6,589,549 titled “Bioactive agent delivering system comprised of microparticles within a biodegradable to improve release profiles” (Macromed, Inc.); U.S. Pat. Nos. 6,007,845 and 5,578,325 titled “Nanoparticles and microparticles of non-linear hydrophilic/hydrophobic multiblock copolymers” (Massachusetts Institute of Technology); US20040234611, US20080305172, US20120269894, and US20130122064 titled “Ophthalmic depot formulations for periocular or subconjunctival administration (Novartis Ag); U.S. Pat. No. 6,413,539 titled “Block polymer” (Poly-Med, Inc.); US 20070071756 titled “Delivery of an agent to ameliorate inflammation” (Peyman); U.S. Pat. No. 6,706,289 titled “Methods and compositions for enhanced delivery of bioactive molecules” (PR Pharmaceuticals, Inc.); and U.S. Pat. No. 8,663,674 titled “Microparticle containing matrices for drug delivery” (Surmodics).

### Dressings and the Like

In some embodiments, the antimicrobial composition containing one or more ammonium silanes or a composition described herein is dispersed in a suitable dressing. The dressing that is chosen should allow release of the desired antimicrobial composition over a period of time dependent upon the desired application. The dressing can be wetted before placement at the site of infection by saturation with the antimicrobial composition, even though it may be additionally moistened due to exudate at the site of infection. Alternatively, the dressing can be placed at the site of a wound and/or infection and subsequently saturated with the antimicrobial composition, for example by application of the antimicrobial composition by dropper or syringe, or other suitable means. In alternative embodiments, the antimicrobial composition containing one or more ammonium silanes or a composition described herein is dispersed in a suitable dressing, wherein at least a portion of the antimicrobial composition remains in order to, for example, reduce odor, to confine the spread or prevent the spread of microbes on the dressing, or to protect a wound or other covering from contamination.

In another embodiment an infection is treated by applying a dressing comprising one or more ammonium silanes described herein as an antimicrobial composition to the site of infection, wherein the dressing releases one or more ammonium silanes described herein into the site of infection. The infection may involve the presence of bacteria, fungi, viruses, amoebas, or a combination of infectious species thereof.

In some embodiments, treatment of an infection comprises placing a dressing comprising an antimicrobial composition as described herein in or on the site of infection.

In certain embodiments, one or more of the ammonium silanes described herein may be used to treat a disorder, typically an infection, caused by gram-positive bacterium, gram-negative bacterium, mycobacterium, fungal species or viral species described herein.

In certain embodiments, a method is provided comprising administering an effective amount of an ammonium silane, or composition thereof described herein to treat an infection caused by gram-positive, gram-negative bacterium, mycobacterium, fungal species or viral species described herein.

In an additional embodiment, a dry powder formulation containing an ammonium silane described herein is impregnated within the dressing and subsequently wetted upon interaction with exudate or other bodily fluids at the site of infection. The dressing may be placed in or on the location of an infection involving a biofilm. The dressing may adhere to the location of a wound and/or infection to provide suitable localization, or may not adhere to the location of the infection in order to prevent undesired tissue damage upon removal. The dressing may be rigid, to allow for it to be held in place during treatment, or may be malleable to allow for placement and adherence in the desired location.

Additionally, the dressing may comprise additional additives that ensure the maintenance of a moist environment at the site of infection. The dressing must be composed of a material that is hypoallergenic and non-toxic in order to be acceptably applied to a living host. In some embodiments, the dressing would absorb the antimicrobial composition and would then subsequently release the antimicrobial composition once placed in the site of infection involving a biofilm.

In certain embodiments, the dressing has a bulk density that is low enough to allow the antimicrobial composition to be incorporated within, but high enough to provide sufficient structural integrity. The dressing should be porous to provide sufficient intercalation of the antimicrobial composition among the material, allowing space for sufficient wetting with the antimicrobial composition along with efflux into the site of treatment. The level of porosity of the dressing should be high enough to allow sufficient wetting with the antimicrobial composition, but should still allow for the dressing to have sufficient material strength.

In certain embodiments, the dressing is fashioned from a flexible polymeric material that provides sufficient porosity but still provides structural integrity in the desired application. In certain embodiments, two or more of the dressing components may be combined, as deemed necessary for the particular application, into a composite material. In some cases, the two or more components may be present in layers. In other cases, the two or more components may be impregnated or intercalated into each other. The combination of dressing components may be necessary for structural integrity to ensure placement, positioning, and functioning at the site of infection.

In some embodiments, the dressing comprises a polymer foam, for example a comformable foam. The polymer foam may allow release of the desired antimicrobial composition by either diffusion, ionic interactions, or by degradation of the material composition of the dressing.

In some embodiments, the polymer foam can absorb exudate that may occur due to infection involving the presence of a biofilm. In some embodiments, the polymer foam is biodegradable or non-degradable, depending upon the intended use within the patient.

Examples of materials suitable for the formation of a polymer foam include, but are not limited to, cellulose and cellulose derivatives, microcrystalline cellulose, calcium alginate, polyacrylic acid, polyethylene glycol, polypropylene glycol, divinyl glycol, polyethylene oxide, polypropylene oxide, carboxymethyl cellulose, hydroxyethyl cellulose, polylactide, polyglycolide, polymethacrylic acid, poly-γ-benzyl-L-glutamate, polypropylene fumarate, poly-F-caprolactone, poly-butylene terephthalate, polyvinyl alcohol, polyvinyl ether, poly-1-vinyl-2-pyrrolidinone, 2,5 dimethyl-1,5-hexadiene, divinyl benzene, polystyrene-divinyl benzene, polyanhydrides such as polybis(p-carboxy-phenoxy)propane-co-sebacic acid, polyhydroxyalkanoates such as poly-β hydroxybutyrate or poly-β-butyrolactone, and alkyl-substituted silica gel formed from reagents such as an ammonium silane and dimethyldiethoxysilane. In preferred embodiments, the polymer foam is composed of polyurethane.

In another embodiment, the polymer foam is composed of cellulose. In yet another embodiment, the polymer foam is composed of calcium alginate.

In some embodiments, the dressing comprises a fabric composition. The fabric composition may be composed of fibers including natural fibers, synthetic fibers, cellulose, woven or nonwoven fabric material, gauze material, or mixtures thereof. Examples of acceptable fibers include, but are not limited to, cotton, polyester, wool, silk, and rayon. The fabric composition may have varying levels of absorbency depending on the desired application. The fabric composition may additionally be coated with an appropriate polymer composition that effects absorbance and dispersion of the active ammonium silane or additional additives.

In some embodiments, the dressing additionally comprises a polymeric film. A polymeric film may be desirable to ensure proper sealing of the dressing to prevent the entry of dirt and debris and to maintain moisture at the treatment site. In preferred embodiments, the polymeric film comprises an adhesive side that adheres to the edges of the site of the infection to provide a seal and a non-adhesive side. In some embodiments, the polymeric film is composed of polyurethane.

In certain embodiments, the dressing is self-adhesive.

In some embodiments, the dressing additionally comprises a collagen matrix. A collagen matrix may be included in applications where it would be deemed desirable, such as providing a template in wound healing. The collagen matrix may be present as an ointment, gel, pad, paste, or sheet. The collagen matrix may be derived from a bovine, porcine, equine, or avian source. The collagen matrix may be composed of type I, II, III, IV, or V collagen.

In some embodiments, the collagen matrix may interact with the site of infection caused by a biofilm by forming a gel. Additional macromolecular structures, such as hyaluronic acid or hyaluronan, fibronectin, laminin, proteoglycans and mixtures thereof, may be incorporated into the collagen matrix. In some embodiments, the collagen matrix is chemically cross-linked.

In some embodiments, the dressing is composed of a dissolvable material. A dressing composed of a dissolvable material can allow for the dressing to be placed in the site of a wound and/or infection without the need for retrieval upon completion of the treatment. In certain embodiments, the dressing comprises a polymeric material comprises thermoplastic polymer, thermosetting polymer, biodegradable polymer, modified polymers, crosslinked polymers, polymers for controlled delivery), hydrogels, hydrocolloids, liquid forming polymer, gel forming polymer, silicone-based polymeric material, film forming polymer, adhesive polymer, polymers for controlled delivery copolymers, polymers for medical uses, or mixtures thereof; fabric material, nonadherent dressing material or hydrofibers.

In certain embodiments, the dressing is hydrofiber. Hydrofibers are soft, sterile, non-woven pad or ribbon dressing composed of sodium carboxymethylcellulose, which is incorporated in the form of a fleece held together by a needle-bonding process. This conformable material can absorb a large amount of wound fluid, such as exudate with bacteria. This is then transformed into a soft gel, which creates a moist environment to support the body's healing process. The gel also aids the removal of non-viable tissue from the wound (autolytic debridement), without damaging newly formed tissue. Hydrofibers are neither hydrocolloids nor alginates, but a separate category incorporating the benefits of both. (Thomas S. Sodium Carboxymethylcellulose Primary Wound Dressing, Aquacel. Available accessed 22 October, 2010)

In certain embodiments, the nonadherent dressing material comprises a nonadherent fabric material, for example, nonadherent gauze, petroleum impregnated gauze, petroleum blend impregnated gauze, oil emulsion dressing, and the like.

In certain embodiments, the dressing comprises polymeric gel (gel forming polymer), silicone, or copolymer thereof, and optionally additional ingredient for the use in topical applications. A polymeric gel may be desirable to ensure proper sealing of the dressing to maintain moisture at the treatment site. In preferred embodiments, the polymeric film comprises an adhesive side that adheres to the edges of the site of the infection to provide a seal and a non-adhesive side.

In some embodiments, the polymeric film is composed of polyurethane. In certain embodiments, the polymeric gel comprises a biodegradable polymer.

In certain embodiments, the polymeric film has a glue-like consistency.

In certain embodiments, the dressing comprises a compound described herein, polymeric gel, silicone, or copolymer thereof, and a pharmaceutically acceptable carrier.

In certain embodiments, the dressing additionally comprises a polymer wherein the polymer can form various shapes, sizes and thicknesses levels suitable for specific applications.

In certain embodiments, the polymer is silicone-based or silicone gel, or silicone-based polymer.

In certain embodiments the polymer is hydrogel, hydrocolloid, or organogel.

In certain embodiments, the polymer is polymeric biomaterial.

In certain embodiments, the polymer is guar gum.

In certain embodiments, the polymer is polyvinyl alcohol.

In certain embodiments, the polymer is hydrogel or cross-linked hydrogel.

In certain embodiments, the polymer is hydrocolloid.

In certain embodiments, the dressing is in the form of a gel, ointment, film, foam, liquid, or hydrocolloid.

In certain embodiments, the polymer is a polyvinyl alcohol; wherein the polymer is a dissolvable polyvinyl alcohol film.

In certain embodiments, the dissolvable polyvinyl alcohol film comprises a silane quaternary compound described herein for the treatment of an infection described herein.

In certain embodiments, the dissolvable polyvinyl alcohol film comprises a silane quaternary compound described herein for the treatment of an infection causing inflammation.

In certain embodiments, the dissolvable polyvinyl alcohol film comprises a silane quaternary compound described herein for the treatment of an ocular infection.

In certain embodiments, the dissolvable polyvinyl film is in the form of an ophthalmic solution or suspension.

In certain embodiments, the dissolvable polyvinyl film is used to coat medical devices for example, catheters and leads and coverings for medical device infections including pacemakers, contact lenses, dentures, prosthetic devices, heart valves or joints, and the like.

In certain embodiments, the dissolvable polyvinyl film is used to treat biofilm; wherein the biofilm is bacterial or fungal. Fungal biofilms on implanted medical devices are highly resistant to drugs and the host immune system and have the potential to seed disseminated blood stream infections. Desai, J., et al., “Fungal Biofilms, Drug Resistance, and Recurrent Infection”, Cold Spring Harb Perspect Med., 2014 4 (10), a019729.

In certain embodiments, the gel, film or hydrocolloid forms a clear stable solution. In certain embodiments, the gel, film or hydrocolloid is stable for at least one month. In certain embodiments, the gel, film or hydrocolloid is stable for at least 2 months. In certain embodiments, the gel, film or hydrocolloid is stable for at least 3 months. In certain embodiments, the gel, film or hydrocolloid is stable for at least 4 months. In certain embodiments, the gel, film or hydrocolloid is stable for at least 5 months. In certain embodiments, the gel, film or hydrocolloid is stable for at least 6 months.

Examples of polymeric gels, include but are not limited to, polyvinyl alcohol, polyvinyl acetate, sodium polyacrylate, acrylate polymers, agarose, galactoarabinan, Poly acrylic acid, polyvinyl chloride, guar gum, xanthan gum, Poly acrylonitrile, polyurethane, Poly(lactide-co-glycolide) (PLGA), Polyethylene glycol (PEG), polyethyleneglycol/dopa, Polycaprolactone (PCL), (poly(vinyl pyrrolidone), poly(ethylene glycol), poly(methyl methacrylate), Poly(N-isopropylacrylamide, polyaniline (PANI), polypyrrole (PPy), polythiophene (PTh), poly(3,4-ethylenedioxythiophene) (PEDOT), polyesters, sulphated polysaccharides (such as heparin, chondroitin, dermatan, keratan sulphates), proplasts or alloplastics, glucan, dextran, derivatives or copolymers thereof.

Examples of polymeric gels include, but are not limited to, Silk sericin, spider silk protein, keratin, hyaluronic acid, pectin, Homoglycans, pallulan, yeast, cellulose, chitin, engineered skin substitutes (single or multilayers), bioengineered skin substitutes (single or multilayers), tissue engineered skin substitutes, dermal substitutes (such as Transcyte, Dermagraft, Composite graft, Orcel, Apligraf, and the like), epidermal substitutes, cultured epidermal autografts (such as Epicel and the like), or acellular xenografts (such as EZ-Derm, Integra, AlloDerm, Derma Martix, and the like) (Mir et al., “Synthetic polymeric biomaterial for wound healing: a review”, Prog Biomater., 2018, 1-21; doi: 10.1007/s40204-018-0083-4; Murray et al., “Development and use of biomaterials as wound healing therapies”, Burns & Trauma, 2019, 7(2), DOI: https://doi.org/10.1186/s41038-018-0139-7)

In certain embodiments, the dressing is used for covering wounds or wound care applications, infection or exposed skin. The wound for example, can be due to an infection, burn, exposed skin, open wound, skin lacerations, abrations, punctures, avulsions, scabs, surgical wounds, abscesses, skin tears, skin ulcers or lesions, damaged tissue, bites, moisture associated skin damage, foot ulcers, necrosis, nonhealing wounds, compromised skin grafts or flaps, acute wounds, chronic wounds, trauma, and other skin exposure related injuries.

Examples include but not limited to, fiber mats gauze (cotton, yarn, natural or synthtetic fibers, nonwoven, blends thereof, and the like), cotton balls, tulle, bandages (liquid bandage), adhesive, tissue adhesive, bioadhesives, tapes, sheets, labels, liners, rolls of film and sheets, tear-apart sheets, rolls, fabric materials described herein, and the like.

In certain applications, the dressing comprises a composite material; wherein the backing materials comprises polyethylene or polypropylene (which can be spun-bonded, spun-laced, or embossed), or a combination thereof. The backing material may for example include one or more materials such as paper, cloth, including medical grade cloth and coated cloth, such as vinyl coated cloth, vinyl, including embossed vinyl, polypropylene, including oriented polypropylene such a MOPP and BOPP, polyesterurethane, polyethylene, including LDPE, LLDPE, MDPE, HMWPE, and HDPE, more generally polyolefin, acrylonitrile butadiene styrene, polycarbonate, polyvinyl chloride, cellophane, cellulose, cellulose acetate, films comprised of block co-polymers such as styrenelisoprene, butadiene or thylene-butylene/styrene (SIS, SBS, or SEBS), polyurethane, ethylene copolymers such as ethylene vinyl acetates, including embossed ethylene vinyl acetate, ethylene/propylene copolymer elastomers or ethylene/propylene/diene terpolymer, nylon, crepe, flat back, a foil, rayon, a polyvinyl derivative, polyamides, cuproammonium cellulose, wool, silk, jute, hemp, cotton, linen, sisal, ramie, polystyrene, polyurethane, polyvinylidene chloride, saponified ethylene-vinyl acetate copolymer, linoleum, acrylics, natural rubber, reclaimed rubber, synthetic rubber, thermoplastic resin films, biodegradable resins such as polylactic acid and polyhydroxyalkanoates, polyamide, polyimide, paper, foil, metalicized films, such as copper film, other resin based films, polylactic acid films, other thin-films, or combinations thereof. Other backing materials may include a fabric material such as one or more woven or non-woven fabric layers, such as non-woven, polyester fabric with a strengthening mesh therein, or a combination thereof. One example is a fabric produced by DuPont E. I. de Nemours & Co., Inc. under the trademark Sontara®.

In certain embodiments, the dressing comprises a composite backing material; wherein the backing material comprises a medical grade cloth material, a polyethylene film material, a vinyl material, an ethylene vinyl acetate material, a polyurethane material, a polyesterurethane material, a polyester material, including a polyethylene terephthalate material, a glycol-modified polyethylene terephthalate (PETG) material, a spun-laced polyethylene terephthalate material, a spun-laced polyethylene terephthalate non-woven material, an embossed non-woven polyethylene terephthalate material, an LDPE non-woven polyethylene terephthalate fabric material, or a coated cloth material, such as a vinyl coated cloth. Non-limiting examples of suitable dissolvable materials for the dressing include poly(lactic acid), polyglycolic acid, poly(caprolactone), poly(3-hydroxybutyrate), poly(s-caproic acid), poly(propylene fumarate), poly(sebaic anhydride), poly(maleic anhydride), poly(ethenol), poly(dioxanone), polyglactin 910, starch or starch derivatives, collagen, chitosan, or mixtures thereof. In some embodiments, the dressing is composed of starch foam that slowly dissolves upon contact with the antimicrobial composition.

In some embodiments, the dressing further comprises one or more additives. The addition of an additive may be necessary to aid in binding of the antimicrobial composition to the dressing or to aid in release of the antimicrobial composition from the dressing into the site of a wound and/or infection. The addition of an additive may also be necessary to favorably change the material properties of the dressing or to enhance binding of the dressing to the site of a wound and/or infection to ensure sufficient delivery of the antimicrobial composition.

In some embodiments, a permeation enhancer is added to the dressing. Permeation enhancers are compounds that increase the level of permeation of the antimicrobial composition provided from the dressing into the layers of the biofilm and any under- or overlaying tissues. Examples of appropriate permeation enhancers include, but are not limited to, ethanol, polyethylene glycol, isopropyl myristate, glycerol trioleate, linolenic acid, glycerol monooleate, glycerol monolaurate, n-decyl alcohol, capric acid, and fatty acid esters, fatty acid alcohols, fatty acid monoglycerides, fatty acid acetates, fatty acid diethanolamides, fatty acid NN-dimethylamides, and combinations thereof.

In some embodiments, a tissue adhesion agent is added to the dressing. In some applications, adhesion of the dressing would be desirable to ensure sufficient delivery of one or more compounds described herein contained in the antimicrobial composition to the biofilm. In many cases, the components of the dressing when impregnated with the antimicrobial composition will have enough adhesion properties to provide sufficient adherence to the tissue containing the infectious biofilm. Such adhesion may not be enough to provide proper support of the dressing on the infected tissue, necessitating the addition of additional tissue adhesion agents. Examples of appropriate adhesion agents include, but are not limited to, hydroxypropylmethylcellulose, carboxymethylcellulose, polylactide-co-glycolide, chitosan, chitosan ester or trimethylenechloride chitosan, sodium alginate, poloxamer, Carbopol, pectin, polyacrylic acid, hyaluronic acid, polyvinyl alcohol, polyvinylpyrrolidone, polycarbophil, and mixtures thereof.

In some embodiments, a plasticizer is added to the dressing. Addition of a plasticizer may be appropriate in order to soften and increase the flexibility of the components of the dressing in certain applications. The improved softness and flexibility increase the number of locations the dressing can be placed within the living host. Some examples of appropriate plasticizers include, but are not limited to, glycerin, water, polyethylene glycol, propylene glycol, sorbitol, and triacetin. Plasticizers are typically added in an amount from about 5% to about 25% by weight.

In certain embodiments, the dressing is designed for placement in a body cavity, for example the external auditory canal so that it might treat infections therein.

In some embodiments, the dressing is of such a size and shape as to fit within the external auditory canal.

In another embodiment, the dressing is malleable such that it can be compressed before placement in the ear, followed by subsequent re-expansion once properly placed.

In another embodiment, the dressing for placement in the external auditory canal is composed of a polymer foam of sufficient porosity to allow intercalation and efflux of the antimicrobial composition described herein.

The compounds described herein may be incorporated in liquid or solid carriers to yield products with antimicrobial properties. The liquid products may be in the form of a lotion.

Compounds of this invention may be added uniformly to thermoplastic polymer products which are extruded (including fibers and tubes) or molded (e.g., supports and scaffolds), or the products may only be protected by coatings containing an ammonium silane described herein. These thermoplastic products may be rigid or flexible; hydrophobic or hydrophilic depending on the dressing required for a given wound. Fibers made using the above process may be converted to yarns, fabrics, scaffolds, etc.; and the ammonium silane-containing product (e.g., fibers) can be combined with other non-antimicrobial-containing product (e.g., fibers) to obtain a final product with a desired level of antimicrobial activity at reduced cost. The materials may also be biocompatible polymers which may remain in the body or decompose to harmless products as healing progresses.

For thermoset polymers the ammonium silane containing materials described herein may be combined with monomeric formulations and then these may be used for molding, casting or coating, etc. The monomers or a part of the monomeric composition may also provide biodegradability to the thermosetting polymer. Curing of any of the thermoset materials/products may be done thermally or by radiation (UV, microwaves, etc).

An ammonium silane of this invention may be added to a variety of solvent (including water)-borne coating formulations, and articles of manufacture coated with these, where the coating is solidified by removing the solvent and/or by curing. One may also fabricate sutures, dental floss, and wound dressings (including burn dressings) using the compounds described herein. Sutures, dressings, or other antimicrobial products and materials used to make final dressings, may consist of fibers, yarns, fabrics, foams, etc. These may be made by incorporating particles containing compounds of this invention into them. One way of such incorporation is to mix a compound of this invention in the polymer and then extrude fibers containing the ammonium silane. These fibers could then be used to make yarns which may be sued as antimicrobial sutures or converted to antimicrobial fabrics for wound dressings or other uses. The antimicrobial fibers may even be converted directly into non-woven fabrics. Antimicrobial fibers may even be mixed with non-antimicrobial fibers to still give an overall antimicrobial character to the products by using these blends. Antimicrobial dressings may also be formed by soaking fibers, yarns, gauze, fabrics and flexible open cell foams in aqueous solutions containing an ammonium silane, removing excess liquids and drying these so that antimicrobial coatings are formed on them. When rigid foams or closed cell foams are used, it is preferred that the antimicrobial material is incorporated into the resin. Products containing antimicrobial materials made by the earlier process may be coated further with additional antimicrobial agents. One may also coat objects using powder coating a well-known technique, where a solid polymeric powder (with an ammonium silane described herein incorporated in this polymeric powder) is applied on an object. The object is then heated to melt the powder to form a coating which is then solidified by curing (due to continued heating or a radiation treatment-such as UV) or by cooling of this coating.

Another area of application is antimicrobial adhesives (including pressure sensitive adhesives). These adhesives may also be biodegradable. These adhesives may be used as a component in the wound dressing or they may be used directly on the wounds. The antimicrobial additives of this invention are preferably added to these when they are in the liquid state.

The ammonium silane of this invention may also be used for dental work. These include applications such as dental adhesives, dental washes and rinses, sutures, primers, sealants and composite fillings and products such as dentures (including antimicrobial solutions to treat dentures), crowns, bridges, epoxies, bone grafts, and coatings including coatings on implants. The methods of incorporating ammonium silanes of this invention in solutions, sealants/adhesives and coatings for dental applications, for example as a cavity cleanser or wash, are very similar to those employed for other applications described herein.

In another example, antimicrobial foams are used in wound dressings so that they would absorb any fluids exuding from the wounds and also ensure that these fluids do not promote colonization of microbes both to prevent infection from spreading and also to act as a deodorant. These antimicrobial foams may be formed by adding an ammonium silane described herein to the monomers or materials which are used to produce this foam, or by first forming the foam, then treating (e.g., soaking and squeezing) the foam with a liquid composition comprising these particles so that they are trapped in the pores or attach to their surfaces.

Other embodiments of products formed from an ammonium silanes described herein include topical creams and liquid suspensions/solutions for both pharmaceutical (e.g., wound care, skin infection care, etc) and consumer product use (e.g., personal care products). They can impart one or both of antimicrobial and/or preservative properties. Preservative property typically means to preserve the product from spoiling under storage conditions-which may go bad due to bacterial and/or fungal growth. The ammonium silanes of this invention may be added to either hydrophilic or hydrophobic cream compositions. As an example, materials compatible with petroleum jelly (a hydrophobic material), may be an appropriate surfactant or a polymer.

The wound dressings may be formed by laminating or combining various layers where each layer provides different functions. A few or all of these layers contain an ammonium silane described herein. The feel or the drape of the dressings and their adhesion properties to the wounds may be modified by adding non-toxic surfactants, glycols, fatty acids and oils, etc. to the compositions containing antimicrobial particles. These dressings may have other medications or additives also incorporated in them (e.g., analgesics) in a post treatment or by adding them to the same solution which contains the ammonium silane. The additives may further include materials which provide enhanced transport of the ammonium silane through the mucus membranes, since the mucus agents form biofilms to protect the bacteria and spores within them. Examples of some materials which penetrate mucus effectively include glucose and xylitol.

A lotion composition for wound management, comprising conventional Povidone-iodine (i.e., PVP-Iodine) could be enhanced by adding an ammonium silane of this invention. As a specific example, aqueous topical solutions of PVP and iodine (where iodine is about 8 to 12% by weight of the PVP) are commonly used as disinfectants for wounds and for disinfecting skin prior to surgery. As an example, BETADINE® is a commercially available PVP-iodine solution. Povidone-iodine (PVP-I) is a stable chemical complex of PVP and elemental iodine. 10% solutions in water are commonly used as a topical antiseptic. Such a metal halide-enhanced PVP-I solution would be formulated having about 88-99% PVP, 2 to 10% Iodine, and 0.005-5% ammonium silane on a wt/wt basis.

Ammonium silanes described herein may also be used as co-additives to other drug/topical formulations including other antibiotic creams or liquid formulations (lotions) for curing or preventing dermal/hair infection control, wound care or related purposes. Some of the typical problems caused by microbial infection relate to acne, athlete's foot, nail infections, dandruff, etc., to just name a few. The antimicrobial materials of this invention may be added in a burn cream, which while assisting the repair of burnt tissue, will also keep infection away, or it may be mixed with other antibiotics, infection reducing/prevention analgesic and wound healing materials such as bacitracin, neomycin, polymyxin, silver sulfadiazine, polyenes, selenium sulfide, zinc pyrithione and paramoxine. Many of these compositions listed above are available in commercial products, and the antimicrobial materials of this invention can be added to them to result in a concentration that is most effective.

Published US patent application 20060269485, incorporated herein by reference, teaches the uses of antibiotic kits which deliver wound care topical materials through aerosol spray and forms a coating (a wound dressing) on the sprayed area.

The ammonium silanes may also contain additional compounds and formulations for inclusion in a wound care product and a wide array of medicinal products. Some examples of medicinal compounds that may be added include, but are not limited to, other antimicrobials, antibiotics, other antifungal agents, other antiviral agents, nutrients (e.g., proteins, carbohydrates, amino acids (such as glutamine, arginine), vitamins (such as A, C and E) and trace elements (such as zinc, iron and magnesium and their compounds)), anti thrombogenic agents, anesthetics, anti-inflammatory agents, analgesics, anticancer agents, vasodilation substances, other wound and tissue healing agents, angiogenic control agents, anti-pruritic agents (anti-itch agents), angiostatic agents, immune boosting agents, growth factors, epithelialization promoting materials (e.g., gentamicin sulfate) and other biological agents. Examples of suitable antimicrobial agents and antibiotics include, but are not limited to, silver preparations (silver and silver compounds (e.g. silver sulfadiazine), in solution or as nanoparticles, silver containing zeolites), elemental iodine, povidone-iodine, biguanide compounds (e.g., polyhexamethylene biguanide), such as chlorhexidine and its salts; triclosan; penicillins; tetracyclines; aminoglycosides, such as gentamicin and Tobramycin™; polymyxins; rifampicins; bacitracins; erythromycins; vancomycins; neomycins; chloramphenicols; neomycin; polyenes; selenium sulfide; zinc pyrithione; paramoxine, maltodextrin; azoles including miconazoles; quinolones, such as oxolinic acid, norfloxacin, nalidixic acid, pefloxacin, enoxacin, and ciprofloxacin; sulfonamides; nonoxynol 9; fusidic acid; cephalosporins; and combinations of such compounds and similar compounds. The additional antimicrobial compounds may provide for enhanced antimicrobial activity. Some natural wound healing materials are acemannan, chitosan, collagen, honey (e.g., medical honey, Manuka honey), sugar, etc.

### Topical Formulations

In certain embodiments, the disclosure provides topical formulations comprising an effective amount of an ammonium silane or composition described herein, or its pharmaceutically acceptable salt, together with at least one topically acceptable carrier for any of the uses described herein. The topical formulation may contain a compound or salt as the only active ingredient, or, in an alternative embodiment, the compound and at least one additional active agent.

Topical formulations are classified into three major categories: solid forms (such as dusting powders); liquid forms (such as lotions and liniments); and semi-liquid forms (such as ointments, pastes, creams, and gels). Additives or excipients are used as inactive ingredients in topical formulations for structuring. Topical formulation additives are mainly used to control the extent of absorption of the active compound, maintaining the viscosity, improving the stability and organoleptic properties, and increasing the bulk of the formulation. A goal of topical formulations is to confine the desired effect to the local area applied with the topical formulation. Such formulations are preferred because they are protective, emollient, and deliver the active agent to exert local activity when applied to, for example, the skin, bone, eye, tissue, or mucous membranes.

In certain embodiments, the topical formulation is a solid formulation such as a dusting powder. A dusting powder is a finely divided insoluble powder containing ingredients used especially for allaying irritation or absorbing moisture, discouraging bacterial growth and providing lubricant properties. Easy powder flow ability and spreadability are important parameters that are considered in the manufacture and evaluation of a dusting powder formulation. The dusting powder should adhere to the area treated, provide good coverage and adsorption, should be free of irritant properties, and should protect the area from drying and irritation. Representative examples of excipients that can be used in dusting powder formulations include, but are not limited to, talc, starch (such as corn starch, wheat starch, or potato starch), kaolin, zinc stearate, zinc oxide, aluminum chlorohydrate, aluminum zirconium chlorhydrex, micronized wax, and chlorhexidine (as the acetate, gluconate, or hydrochloride salt).

In certain embodiments, the topical formulation is a cream formulation. Creams are semisolid emulsion formulation for application to the skin or mucous membranes. Creams may be formulated as water in oil (w/o) emulsions or as oil in water (o/w) emulsions. Water in oil emulsion creams are less greasy and provide good spreadability compared to ointments. Oil in water emulsion creams, often called vanishing creams, readily rub into the skin and are easily removed by water.

Water in oil emulsion formulations typically consist of a hydrophilic component, e.g., water or other hydrophilic diluent, and a hydrophobic component, e.g., a lipid, oil, or oily material. The hydrophilic component is typically dispersed, i.e., exists as small particles and droplets, within the hydrophobic component. Water in oil emulsions typically comprise from about 1% to about 98% of the dispersed hydrophilic phase and from about 1% to about 50% of the hydrophobic phase. Additives commonly used in water in oil emulsion formulations include wool fat (containing sterols, cholesterol, oxycholesterol, triterpene, or aliphatic alcohols), waxes, bivalent soaps, sorbitan esters, borax, and oleic acid. In some embodiments, the water in oil emulsion refers to a water in silicone emulsion.

Oil in water emulsion formulations typically consist of a hydrophilic component, e.g., water or other hydrophilic diluent, and a hydrophobic component, e.g., a lipid, oil, or oily material. The hydrophobic component is typically dispersed, i.e., exists as small particles and droplets, within the hydrophilic component. Water in oil emulsions typically comprise from about 1% to about 98% of the hydrophilic phase and from about 1% to about 50% of the dispersed hydrophobic phase. Additives commonly used in oil in water emulsion formulations include polysorbates (such as Tween 80, Tween 21, and Tween 40), methylcellulose, acacia, tragacanth, triethanolamine oleate, arachis oil, and cetostearyl alcohol.

In certain embodiments, the topical formulation is an ointment formulation. Ointments are greasy semisolid preparations of a dissolved or dispersed active compound. Ointment bases often influence topical drug bioavailability due to their occlusive properties of the stratum corneum, which enhances the flux of drug across the skin and affects drug dissolution or partitioning within and from the ointment to the skin. Ointments usually are moisturizing and are good for dry skin, as well as having a low risk of sensitization or irritation due to having few ingredients beyond the base oil or fat. The vehicle for an ointment formulation, known as an ointment base, may be an oleaginous base, an absorption base, or a water-soluble base.

Oleaginous bases are composed entirely of lipophilic materials. They are anhydrous, insoluble in water, and not easily removable with water. Oleaginous bases are inexpensive, non-reactive, nonirritating, are good emollients, have protective and occlusive properties, and are not water washable. Representative examples of oleaginous bases include hydrocarbons (such as petrolatum, paraffin wax, liquid paraffin, microcrystalline wax, plastibase, or Ceresi), vegetable oils and animal fat (such as coconut oil, bees wax, olive oil, lanolin, peanut oil, spermacetic wax, sesame oil, or almond oil), hydrogenated and sulfated oils (such as hydrogenated castor oil, hydrogenated cotton seed oil, hydrogenated soya bean oil, hydrogenated corn oil, or hydrogenated sulfated castor oils), alcohols/acids/esters (such as cetyl alcohol, stearic acid, stearyl alcohol, oleic acid, olelyl alcohol, palmitic acid, lauryl alcohol, lauraic acid, myristyl alcohol, ethyl oleate, isopropyl myristicate, or ethylene glycol), and silicones (such as dimethylpropylsiloxanes, methyl phenyl polysiloxanes, and steryl esters of dimethyl polysiloxanes).

Absorption bases are known to take up several times their own weights in water but not permit absorption of medicament form the base. The advantages of absorption bases are their protective, occlusive, and emollient properties, their ability to absorb liquids, and that they do not wash off easily so they hold the incorporated compound with sufficient contact with the skin. Representative examples of absorption bases include hydrophilic petrolatum and anhydrous lanolin.

Water-soluble bases, also known as greaseless ointment bases, consists of water-soluble ingredients such as polyethylene glycol polymer (carbowax). Polyethylene glycol is water soluble, nonvolatile, and inert. Other water-soluble bases include glyceryl monostearate, cellulose derivatives, sodium alginate, bentonite, and carbopol 934.

In certain embodiments, the topical formulation is a gel formulation. Gels are transparent or translucent semisolid preparations of one or more active ingredients in suitable hydrophilic or hydrophobic bases. Gels may be clear or opaque, and polar hydroalcoholic or nonpolar. Gels are prepared by either a fusion process or a special procedure necessitated by the gelling agents, humectants, and preservatives. Gelling agents exhibit pseudoplastic properties that give the formulation a thixotropic consistency. Gelling agents are typically used in concentrations of 0.5-10% to allow for easy addition of the active drug before the gel is formed. Representative examples of agents used in gel formulations include tragacanth, fenugreek mucilage, methyl cellulose, hydroxy ethyl cellulose, hydroxy propyl cellulose, hydroxy propyl methyl cellulose, carboxy methylcellulose, carbopol, pectin, poloxamers, alginates (such as sodium, potassium, or ammonium alginates), gelatin, starch, polyvinyl alcohol, povidone, propylene glycol, and ethyldiamine tetraacetic acid.

In some embodiments, the topical formulation is a paste formulation. Pastes are stiff preparations containing a high proportion of a finely powdered solid such as starch, zinc oxide, calcium carbonate, or talc. Pastes are often less greasy than ointment formulations. In some embodiments, the topical formulation is in the form of a toothpaste, further comprising for example, abrasives, fluoride, and optionally other antibacterial agents such as triclosan.

In some embodiments, the topical formulation is a lotion formulation. Lotions are low- to medium-viscosity preparations intended for application to unbroken skin. Lotions are applied to external skin with bare hands, a clean cloth, cotton wool or gauze. Lotions provide cooling effects to the skin by the evaporation of solvents formulated therein. Typical additives in lotion formulations include bentonite, sodium carboxymethylcellulose, alcohols, and glycerin.

In some embodiments, the topical formulation is a liniment formulation. Liniments are liquid or semiliquid preparations meant for application to the skin with friction or rubbing. They act as a rubefacient, soother, or stimulant. Typical vehicles for liniment formulations are alcohol, oil, or soap based. Typical additives in a liniment formulation include castor oil, cotton seed oil, peanut oil, sesame oil, and oleic acid.

A wide variety of optional components/ingredients may be included in the topical formulations including, but not limited to, absorbents, abrasives, anticaking agents, antifoaming agents, antimicrobial agents, binders, biological actives, buffering agents, bulking agents, chemical additives, cosmetic biocides, denaturants, cosmetic astringents, drug astringents, external analgesics, film formers, humectants, opacifying agents, fragrances, pigments, colorings, essential oils, skin sensates, emollients, skin soothing agents, skin healing agents, pH adjusters, plasticizers, preservatives, preservative enhancers, propellants, reducing agents, additional skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, oil/sebum control agents, sweat control agents, sequestrants, anti-inflammatory agents, anti-androgens, depilation agents, desquamation agents/exfoliants, organic hydroxy acids, vitamins and derivatives thereof, and natural extracts.

The present method includes identifying a target portion of skin affected with acne vulgaris and in need of treatment and applying a compound or its salt or composition as described herein to the target portion of skin. In certain embodiments, the target portion of skin may not appear to be suffering from acne vulgaris, i.e., the compound or its salt or composition as described herein may be used as a preventative therapy for acne vulgaris. The compound or its salt or composition may be applied to the target skin portion and, if desired, to the surrounding skin at least once a day, twice a day, or on a more frequent daily basis during the treatment period. Typically, the compound or its salt or composition is applied in the morning and/or in the evening before bed.

The treatment period is ideally sufficient time for the active compound to reduce or eliminate the appearance of acne vulgaris on the target portion of skin. The treatment period may last for at least 1 week, about two weeks, about 4 weeks, about 8 weeks, or about 12 weeks. The treatment period may extend over multiple months (about 3-12 months) or multiple years. The step of applying the compound or its salt or composition may be accomplished by localized application, i.e. by applying to the targeted area while minimizing delivery to skin surfaces where treatment is not desired, or by applying more generally or broadly to one or more skin surfaces.

### Microparticles and Nanoparticles for Use in the Present Invention

In some embodiments, an ammonium silane or composition described herein is provided in the form of a microparticle or nanoparticle. The desired microparticles or nanoparticles can be formed using a method to provide pharmaceutically suitable microparticles.

In some embodiments, the microparticles or nanoparticles are dispersed in water or other pharmaceutically appropriate carrier.

In some embodiments, the microparticles or nanoparticles allow controlled release of the desired antimicrobial agent by slow dissolution of one or more ammonium silanes described herein in the chosen carrier or in the moisture present at the site of infection.

In some embodiments, the microparticles or nanoparticles are combined with an appropriate polymer matrix for use during processing. The appropriate polymer matrix is chosen such that the rate of dissolution of one or more ammonium silanes described herein into the carrier is controlled at the site of infection.

In another embodiment, the microparticles or nanoparticles are intercalated within a dressing as described herein.

In another embodiment, the microparticles or nanoparticles are intercalated within a compound used to promote bone or tissue regeneration.

Microparticles and nanoparticles can be formed using any suitable method for the formation of microparticles known in the art. Microparticles assembled with two-dimensional nanostructures, such as CNT's, can be equipped with improved mechanical and electrical properties without sacrificing permeability at the molecular level to assist in the desired placement of the compound (Kim, M., Choi, M. G., Ra, H. W., Park, S. B., Kim, Y.-J., & Lee, K. (2018). Encapsulation of Multiple Microalgal Cells via a Combination of Biomimetic Mineralization and LbL Coating. Materials, 11(2), 296. http://doi.org/10.3390/ma11020296).

The method employed for particle formation will depend on a variety of factors, including the characteristics of one or more ammonium silanes described herein, as well as the desired particle size and size distribution. The type of compound being incorporated into the microparticles may also be a factor as some compositions are unstable in the presence of certain solvents, in certain temperature ranges, or in certain pH ranges.

Particles having an average particle size of between about 1 micron and 100 microns are useful as microparticles in the compositions described herein. In typical embodiments, the particles have an average particle size of between about 1 micron and 40 microns, more typically between about 10 microns and about 40 microns, more typically between about 20 microns and about 40 microns. The particles can have any shape but are generally spherical in shape.

Particles having an average particle size of between about 1 and 100 nanometers (nm) are useful as nanoparticles in the compositions described herein. In typical embodiments, the particles have an average particle size of between about 1 nm and 75 nm, more typically between about 10 microns and about 40 microns, more typically between about 20 microns and about 40 microns. The particles can have any shape but are also generally spherical in shape.

Nanoparticles are useful for delivery through mucosal barriers where very small particles have an advantage of easier travel through the mucus than larger particles. This is useful for ocular delivery, as the eye is covered with a mucosal barrier.

In circumstances where a monodispersed population of particles is desired, the particles may be formed using a method which produces a monodisperse population of microparticles. Alternatively, methods producing polydispersed microparticle distributions can be used, and the particles can be separated using methods known in the art, such as sieving, following particle formation to provide a population of particles having the desired average particle size and particle distribution.

Common techniques for preparing microparticles and nanoparticles include, but are not limited to, solvent evaporation, hot melt particle formation, solvent removal, spray drying, phase inversion, coacervation, and low temperature casting. Suitable methods of particle formation are briefly described below. Pharmaceutically acceptable excipients, including pH modifying agents, disintegrants, preservatives, and antioxidants, can optionally be incorporated into the particles during particle formation.

In some embodiments, the desired microparticles and nanoparticles are obtained through a solvent evaporation method. In one aspect, a method is provided whereby one or more ammonium silanes described herein or polymer matrix and one or more compounds described herein, is dissolved or dispersed in a volatile organic solvent, such as methylene chloride, acetone, acetonitrile, 2-butanol, 2-butanone, t-butyl alcohol, benzene, chloroform, cyclohexane, 1,2-dichloroethane, diethyl ether, ethanol, ethyl acetate, heptane, hexane, methyl tert-butyl ether, pentane, petroleum ether, isopropyl alcohol, n-propanol, tetrahydrofuran, or mixtures thereof. The organic solution containing one or more ammonium silanes described herein is then suspended in an aqueous solution that contains a surfactant, such as poly(vinyl alcohol). The resulting emulsion is stirred until most of the organic solvent is evaporated, leaving solid microparticles. The resulting microparticles are rinsed with water and dried in a lyophilizer overnight. Microparticles with different sizes and morphologies can be obtained with this method.

Solvent removal can be used to prepare particles from ammonium silanes described herein that are deemed hydrolytically unstable. In one aspect, a method is provided whereby one or more ammonium silanes described herein are dissolved or dispersed in a volatile organic solvent such as methylene chloride, acetone, acetonitrile, 2-butanol, 2-butanone, t-butyl alcohol, benzene, chloroform, cyclohexane, 1,2-dichloroethane, diethyl ether, ethanol, ethyl acetate, heptane, hexane, methyl tert-butyl ether, pentane, petroleum ether, isopropyl alcohol, n-propanol, tetrahydrofuran, or mixtures thereof. This mixture is then suspended by stirring in an organic oil (such as silicon oil, castor oil, paraffin oil, or mineral oil) to form an emulsion. Solid particles form from the emulsion, which can subsequently be isolated from the supernatant. The external morphology of spheres produced with this technique is highly dependent upon the identity of one or more ammonium silanes described herein.

In some embodiments, the microparticles are formed by using an oil-in-water emulsion. In one aspect, a method is provided whereby, one or more ammonium silanes described herein are dissolved or dispersed in a volatile organic solvent such as methylene chloride, acetone, acetonitrile, 2-butanol, 2-butanone, t-butyl alcohol, benzene, chloroform, cyclohexane, 1,2-dichloroethane, diethyl ether, ethanol, ethyl acetate, heptane, hexane, methyl tert-butyl ether, pentane, petroleum ether, isopropyl alcohol, n-propanol, tetrahydrofuran, or mixtures thereof. This mixture is then suspended by stirring in an aqueous solution of a surfactant to form an emulsion. Solid particles form from the emulsion, which can subsequently be isolated from the supernatant. The external morphology of the spheres produced with this technique is highly dependent upon the identity of one or more ammonium silanes described herein.

In some embodiments, the microparticles are derived by spray drying. In one aspect, a method is provided whereby, one or more ammonium silanes described herein are dissolved in an organic solvent such as methylene chloride, acetone, acetonitrile, 2-butanol, 2-butanone, t-butyl alcohol, benzene, chloroform, cyclohexane, 1,2-dichloroethane, diethyl ether, ethanol, ethyl acetate, heptane, hexane, methyl tert-butyl ether, pentane, petroleum ether, isopropyl alcohol, n-propanol, tetrahydrofuran, or mixtures thereof. The mixture is pumped through a micronizing nozzle driven by a flow of compressed gas, and the resulting aerosol is suspended in a heated cyclone of air, allowing the solvent to evaporate from the microdroplets, forming particles. Particles ranging between 0.1-10 microns can be obtained using this method.

Particles can be formed from one or more ammonium silanes described herein using a phase inversion method. In one aspect a method is provided whereby one or more ammonium silanes described herein are dissolved in a solvent, and the solution is poured into a strong non-solvent for the substrate to spontaneously produce, under favorable conditions, microparticles. The method can be used to produce particles in a wide range of sizes, including, for example, from about 100 nanometers to about 10 microns, typically possessing a narrow particle size distribution.

In some embodiments, the particles can be formed from one or more ammonium silanes described herein using coacervation. Techniques for particle formation using coacervation are known in the art, for example, in GB-B-929 406; GB-B-929 50 1; and U.S. Pat. Nos. 3,266,987, 4,794,000, and 4,460,563.

In some embodiments, the particles can be formed from one or more ammonium silanes described herein by low temperature casting. Methods for very low temperature casting of microspheres are described in U.S. Pat. No. 5,019,400 to Gombotz et al. In one aspect, a method is provided whereby one or more ammonium silanes described herein is dissolved in an appropriate solvent. The mixture is then atomized into a vessel containing a liquid non-solvent at a temperature below the freezing point of the QAC solution which freezes the droplets. As the droplets and the non-solvent for the QAC are warmed, the solvent in the droplets thaws and is extracted into the non-solvent, hardening the microspheres.

In certain embodiments, one or more ammonium silanes described herein can be incorporated into a polymeric nanoparticle, e.g. for convenience of delivery, targeted delivery or extended release delivery. The use of materials in nanoscale provides one the ability to modify fundamental physical properties such as solubility, diffusivity, blood circulation half-life, release characteristics of one or more ammonium silanes described herein, or immunogenicity. A number of nanoparticle-based therapeutic and diagnostic agents have been developed for the treatment of cancer, diabetes, pain, asthma, allergy, and infections. These nanoscale agents may provide more effective or more convenient routes of administration, lower therapeutic toxicity, extend the product life cycle, and ultimately reduce health-care costs. As therapeutic delivery systems, nanoparticles can provide targeted delivery and controlled release.

In addition, nanoparticle-based delivery can be used to release one or more ammonium silanes described herein at a sustained rate and thus lower the frequency of administration, deliver one or more ammonium silanes described herein in a targeted manner to minimize side effects, or to deliver one or more ammonium silanes described herein and an additional pharmaceutical active simultaneously for combination therapy to generate a synergistic effect and suppress drug resistance. Among these products, liposomal drugs and polymer-based conjugates account for a large proportion of the products. See, Zhang, L., et al., Nanoparticles in Medicine: Therapeutic Applications and Developments, Clin. Pharm. and Ther., 83(5):761-769, 2008.

Methods for producing nanoparticles are known in the art. For example, see Muller, R. H., et al., Solid lipid nanoparticles (SLN) for controlled drug delivery—a review of the state of the art, Eur. H. Pharm. Biopharm., 50:161-177, 2000; U.S. Pat. No. 8,691,750 to Consien et al.; WO 2012/145801 to Kanwar. U.S. Pat. No. 8,580,311 to Armes, S. et al.; Petros, R. A. and DeSimone, J. M., Strategies in the design of nanoparticles for therapeutic applications, Nature Reviews/Drug Discovery, vol. 9:615-627, 2010; U.S. Pat. Nos. 8,465,775; 8,444,899; 8,420,124; 8,263,129; 8,158,728; 8,268,446; Pellegrino et al., 2005, Small, 1:48; Murray et al., 2000, Ann. Rev. Mat. Sci., 30:545; and Trindade et al., 2001, Chem. Mat., 13:3843; all incorporated herein by reference. Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)), U.S. Pat. No. 5,543,158 to Gref et al., or WO publication WO2009/051837 by Von Andrian et al. Zauner et al., 1998, Adv. Drug Del. Rev., 30:97; and Kabanov et al., 1995, Bioconjugate Chem., 6:7; (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372; Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633; U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et 84 al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181; Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732; C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010); U.S. Pat. No. 6,632,671 to Unger Oct. 14, 2003, all incorporated herein by reference.

### Combination Therapy

In some embodiments, an ammonium silane or composition as described herein may be provided in combination or alternation with or preceded by, concomitant with or followed by, an effective amount of at least one additional therapeutic agent, for example, for treatment of a disorder described herein.

Non-limiting examples of additional active agents for such combination therapy are provided below. In the described below and herein generally, whenever any of the terms referring to an ammonium silane, or composition as described herein are used, it should be understood that salts, prodrugs, or compositions are considered included, unless otherwise stated or inconsistent with the text.

In some embodiments, an ammonium silane, or composition as described herein may be used in combination or alternation with an antibiotic.

In some embodiments, the antibiotic is an aminoglycoside. In some embodiments, the antibiotic is selected from amikacin, gentamicin, kanamycin, neomycin, netilmicin, tobramycin, paromomycin, streptomycin, and spectinomycin.

In some embodiments, the antibiotic is anansamycin. In some embodiments, the antibiotic is selected from geldanamycin, herbimycin, and rifaximin.

In some embodiments, the antibiotic is a carbapenem. In some embodiments, the antibiotic is selected from ertapenem, doripenem, imipenem, panipenem, biapenem, tebipenem, and meropenem.

In some embodiments, the antibiotic is a cephalosporin. In some embodiments, the antibiotic is selected from cefacetrile, cefadroxil, cephalexin, cefaloglycin, cefalonium, cefaloridine, cefalotin, cefapirin, cefatrizine, cefazaflur, cefazedone, cefazolin, cefradrine, cefroxadine, and ceftezole. In some embodiments, the antibiotic is selected from cefaclor, cefonicid, cefprozil, cefuroxime, cefuzonam, cefmetazole, cefotetan, cefoxitin, loracarbef, cefbuperazone, cefminox, cefoxitin, and cefotiam. In some embodiments, the antibiotic is selected from cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, and latamoxef. In some embodiments, the antibiotic is selected from cefclidine, cefepime, cefluprenam, cefoselis, cefozopran, cefpirome, cefquinome, and flomoxef. In some embodiments, the antibiotic is selected from ceftobiprole, ceftaroline, and ceftolozane.

In some embodiments, the antibiotic is a glycopeptide. In some embodiments, the antibiotic is selected from teicoplanin, vancomycin, telavancin, dalbavancin, ramoplanin, decaplanin, and oritavancin.

In some embodiments, the antibiotic is a lincosamide. In some embodiments, the antibiotic is selected from lincomycin, clindamycin, and pirlimycin.

In some embodiments, the antibiotic is daptomycin. In some embodiments, the antibiotic is a macrolide. In some embodiments, the antibiotic is selected from azithromycin, clarithromycin, erythromycin, fidaxomicin, telithromycin, carbomycin A, josamycin, kitasamycin, midecamycin, oleandomycin, solithromycin, spiramycin, troleandomycin, tylosin, and roxithromycin.

In some embodiments, the antibiotic is a ketolide. In some embodiments, the antibiotic is selected from telithromycin, cethromycin, and solithromycin. In some embodiments, the antibiotic is a monobactam.

In some embodiments, the antibiotic is selected from aztreonam. In some embodiments, the antibiotic is a nitrofuran. In some embodiments, the antibiotic is selected from diruazone, furazolidone, nifurfoline, nifuroxazide, nifurquinazol, nifurtoinol, nifurzide, nitrofural, and nitrofurantoin.

In some embodiments, the antibiotic is an oxazolidinone. In some embodiments, the antibiotic is selected from linezolid, posizolid, tedizolid, radezolid, torezolid, and cycloserine.

In some embodiments, the antibiotic is a penicillin. In some embodiments, the antibiotic is selected from penicillin G, penicillin K, penicillin N, penicillin O, and penicillin V. In some embodiments, the antibiotic is selected from meticillin, nafcillin, oxacillin, cloxacillin, dicloxacillin, and flucoxacillin. In some embodiments, the antibiotic is selected from ampicillin, amoxicillin, pivampicillin, hetacillin, bacampicillin, metampicillin, talampicillin, and epicillin. In some embodiments, the antibiotic is selected from carbenicilin, ticarcillin, and temocillin. In some embodiments, the antibiotic is selected from mezlocillin and piperacillin. In some embodiments, the antibiotic is selected from clavulanic acid, sulbactam, and tazobactam.

In some embodiments, the antibiotic is a polypeptide antibiotic. In some embodiments, the antibiotic is selected from bacitracin, colistin, and polymyxin B.

In some embodiments, the antibiotic is a quinolone or fluoroquinolone antibiotic. In some embodiments, the antibiotic is selected from flumequine, oxolinic acid, rosoxacin, cinoxacin, nalidixic acid, and piromidic acid. In some embodiments, the antibiotic is selected from ciprofloxacin, fleroxacin, lomefloxacin, nadifloxacin, norfloxacin, ofloxacin, pefloxacin, rufloxacin, and enoxacin. In some embodiments, the antibiotic is selected from balofloxacin, grepafloxacin, levofloxacin, pazufloxacin, sparfloxacin, temafloxacin, and tosufloxacin. In some embodiments, the antibiotic is selected from clinafloxacin, gatifloxacin, moxifloxacin, sitafloxacin, prulifloxacin, besifloxacin, gemifloxacin, trovafloxacin, delafloxacin, and ozenoxacin.

In some embodiments, the antibiotic is a sulfonamide. In some embodiments, the antibiotic is selected from sulfacetamide, sulfadiazine, sulfadimidine, sulfafurazole, sulfisomidine, sulfadoxine, sulfamethoxazole, sulfamoxole, sulfanitran, sulfadimethoxine, sulfamethoxypyridazine, sulfametoxydiazine, sulfadoxine, sulfametopyrazine, terephtyl, mafenide, sulfanilamide, sulfasalazine, sulfisoxazole, and sulfonamicochrysoidine.

In some embodiments, the antibiotic is a tetracycline. In some embodiments, the antibiotic is selected from tetracycline, chlortetracycline, oxytetracycline, demeclocycline, lymecycline, meclocycline, metacycline, minocycline, and rolitetracycline.

In some embodiments, the antibiotic is selected from clofazimine, dapsone, capreomycin, cycloserine, ethambutol, ethionamide, isoniazid, pyrazinamide, rifampicin, rifabutin, rifapentine, and streptomycin. In another embodiment, the antibiotic is selected from arsphenamide, chloramphenicol, fosfomycin, fusidic acid, metronidazole, mupirocin, platensimycin, quinupristin, dalfopristin, thiamphenicol, tigecycline, and trimethoprim.

In some embodiments, an ammonium silane, or composition as described herein may be used in combination or alternation with an antifungal drug.

In some embodiments, the antifungal drug is an azole antifungal. In some embodiments, the antifungal drug is selected from bifonazole, butoconazole, clotrimazole, econazole, fenticonazole, isoconazole, ketoconazole, luliconazole, miconazole, omoconazole, oxiconazole, sertaconazole, sulconazole, and tioconazole. In some embodiments, the antifungal drug is selected from albaconazole, efinaconazole, epoxiconazole, fluconazole, isavuconazole, itraconazole, posaconazole, propiconazole, ravuconazole, terconazole, and voriconazole.

In some embodiments, the antifungal drug is abafungin. In some embodiments, the antifungal drug is an echinocandin. In some embodiments, the antifungal drug is selected from anidulafungin, caspofungin, and micafungin.

In some embodiments, the antifungal drug is a polyene antifungal. In some embodiments, the antifungal drug is selected from amphotericin B, candicidin, filipin, hamycin, natamycin, nystatin, and rimocidin. In some embodiments, the antifungal drug is selected from griseofulvin, terbinafine, and flucytosine.

In certain embodiments, an ammonium compound or composition described herein, or its salt may be used in combination or alternation with benzoyl peroxide. In the skin follicle, benzoyl peroxide kills P. acnes by oxidizing its proteins through the formation of oxygen free radicals and benzoic acid. These radicals are believed to interfere with the bacterium's metabolism and ability to make proteins. Additionally, benzoyl peroxide is mildly effective at breaking down comedones and inhibiting inflammation. In some embodiments, an active compound or its salt is formulated in combination with benzoyl peroxide in a topical formulation as described herein.

In certain embodiments, an ammonium compound or composition described herein, or its salt may be used in combination or alternation with a retinoid. Retinoids are medications which reduce inflammation, normalize the follicle cell life cycle, and reduce sebum production. They are structurally related to vitamin A. The retinoids appear to influence the cell life cycle in the follicle lining; this helps prevent the accumulation of skin cells within the hair follicle that can create a blockage. Frequently used topical retinoids include adapalene, isotretinoin, retinol, tazarotene, and tretinoin. In some embodiments, an active compound or its salt is formulated in combination with a retinoid in a topical formulation as described herein.

In certain embodiments, an ammonium compound or composition described herein, or its salt may be used in combination or alternation with an antibiotic. Antibiotics are frequently applied to the skin or taken orally to treat acne and are thought to work due to their antimicrobial activity against P. acnes and their ability to reduce inflammation. Commonly used antibiotics, either applied to the skin or taken orally, include clindamycin, erythromycin, metronidazole, sulfacetamide, and tetracyclines such as doxycycline and minocycline. Other representative topical antibiotics include bacitracin, polymycin b, neomycin, retapamulin, mupirocin, pramoxine, gentamicin, mafenide, and ozenoxacin. The compounds described herein are particularly effective in combination with antibiotics due to their potentiation of the antimicrobial effect of the antibiotic. In some embodiments, an active compound or its salt is formulated in combination with an antibiotic in a topical formulation as described herein.

In certain embodiments, an ammonium compound or composition described herein, or its salt may be used in combination or alternation with azelaic acid. Azelaic acid is thought to be an effective acne treatment due to its ability to reduce skin cell accumulation in the follicle, along with its antibacterial and anti-inflammatory properties. In some embodiments, an active compound or its salt is formulated in combination with an antibiotic in a topical formulation as described herein.

In certain embodiments, an ammonium compound or composition described herein, or its salt may be used in combination or alternation with salicyclic acid. Salicyclic acid is a topically applied beta-hydroxy acid that has keratolytic properties in addition to stopping bacterial reproduction. In some embodiments, an active compound or its salt is formulated in combination with salicyclic acid in a topical formulation as described herein.

In certain embodiments, an ammonium compound or composition described herein, or its salt may be used in combination or alternation with niacinamide. Niacinamide can improve acne by decreasing inflammation, suppressing sebum production, and promoting wound healing. In some embodiments, an active compound or its salt is formulated in combination with salicyclic acid in a topical formulation as described herein.

### REPRESENTATIVE EXAMPLES OF THE PRESENT INVENTION

Abbreviations in the examples below are defined as follows: DMF is dimethylformamide, MeOH is methanol, and NMR is nuclear magnetic resonance spectroscopy. N-Tris(hydroxymethyl)methyl-2-aminoethanesulfonic acid is abbreviated herein as TES. Tris(hydroxymethyl)aminomethane is abbreviated as TRIS. In the examples below, a skilled artisan can remove and substitute compounds with Na+ and Cl— charges with other salts.

### Example 1. Synthesis of 2-[[1-[[3-[dimethyl(octadecyl)ammonio]propyl-[3-hydroxy-2-(hydroxymethyl)-2-(2-sulfoethylamino)propoxy]-[3-hydroxy-2-(hydroxymethyl)-2-(2-sulfoethylamino)propoxy]silyl]oxymethyl]-2-hydroxy-1-(hydroxymethyl)ethyl]amino]-ethanesulfonate

A 500 mL round bottom reaction vessel was outfitted with an oil bath, stir bar, distillation head and condenser, receiving flask, oil bubbler, gas inlet that allows argon to pass through vessel and distillation setup, an immersion thermoprobe for the oil bath, and a thermoprobe for the head. The reactor is charged with 47.6 mL of a 42% methanolic solution of dimethyloctadecyl[3-(trimethoxysilyl)propyl]ammonium chloride (Aldrich), 200 mL DMF, 27.7 g (3 equiv.) of N-Tris(hydroxymethyl)methyl-2-aminoethanesulfonic acid (TES) and 8.7 mL of 25% MeONa in MeOH. The mixture is heated to 125 degrees C. (oil temp) and the methanol is collected by distillation. As the reaction is heated a homogenous solution is formed. As the methanol is distilled off a white precipitate begins to form. The reaction is heated until the head temp drops and no more MeOH is evolved (about 1.5 hours). The reaction is then cooled to room temperature and the precipitate (NaCl) is removed by filtration through a glass fiber filter and the DMF was concentrated to about 100 mL total volume at reduced pressure. The solution is cooled to rt. A 1 L flask under argon and a stir bar is charged with anhydrous acetonitrile (1000 mL) and cooled to ˜10 C. The DMF solution is added slowly to rapidly stirring acetonitrile to form a white suspension. Stirring is continued for 30 minutes. The colorless solid is collected on a glass frit under argon and washed with acetonitrile and then ether. The hygroscopic solid is then transferred to a vacuum flask and dried under vacuum. The resulting fine powder dissolves quickly in water with gentle heating. Initially it is hazy but slowly clears. A 1% solution in water remains clear for >5 months.

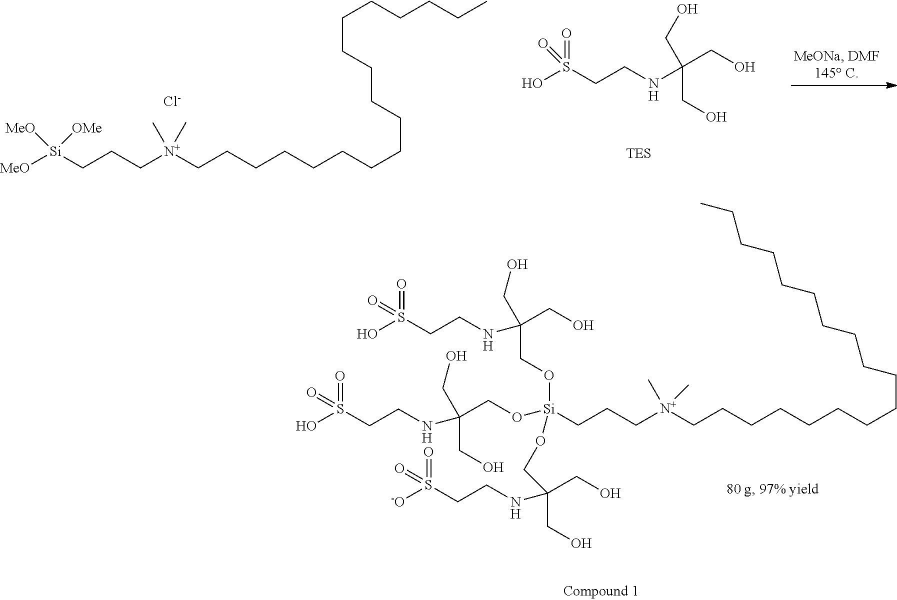

### Example 2. Synthesis of 2-[[1-[[3-[dimethyl(octadecyl)ammonio]propyl-[3-hydroxy-2-(hydroxymethyl)-2-(2-sulfoethylamino)propoxy]-[3-hydroxy-2-(hydroxymethyl)-2-(2-sulfoethylamino)propoxy]silyl]oxymethyl]-2-hydroxy-1-(hydroxymethyl)ethyl]amino]-ethanesulfonate

A 500 mL round bottom reaction vessel was outfitted with an oil bath, stir bar, distillation head and condenser, receiving flask, oil bubbler, gas inlet that allows argon to pass through vessel and distillation setup, an immersion thermoprobe for the oil bath, and a thermoprobe for the head. The reactor is charged with 47.6 mL of a 42% methanolic solution of dimethyloctadecyl[3-(trimethoxysilyl)propyl]ammonium chloride (Aldrich), 200 mL DMF, 27.7g (3 equiv.) of N-Tris(hydroxymethyl)methyl-2-aminoethanesulfonic acid (TES) and 8.7 mL of 25% EtONa in EtOH. The mixture is heated to 125 degrees C. (oil temp) and the methanol is collected by distillation. As the reaction is heated a homogenous solution is formed. As the methanol is distilled off a white precipitate begins to form. The reaction is heated until the head temp drops and no more MeOH is evolved (about 1.5 hours). The reaction is then cooled to room temperature and the precipitate (NaCl) is removed by filtration through a glass fiber filter and the DMF was concentrated to about 100 mL total volume at reduced pressure. The solution is cooled to rt. A 1 L flask under argon and a stir bar is charged with anhydrous acetonitrile (1000 mL) and cooled to ˜10 C. The DMF solution is added slowly to rapidly stirring acetonitrile to form a white suspension. Stirring is continued for 30 minutes. The colorless solid is collected on a glass frit under argon and washed with acetonitrile and then ether and dried under vacuum.

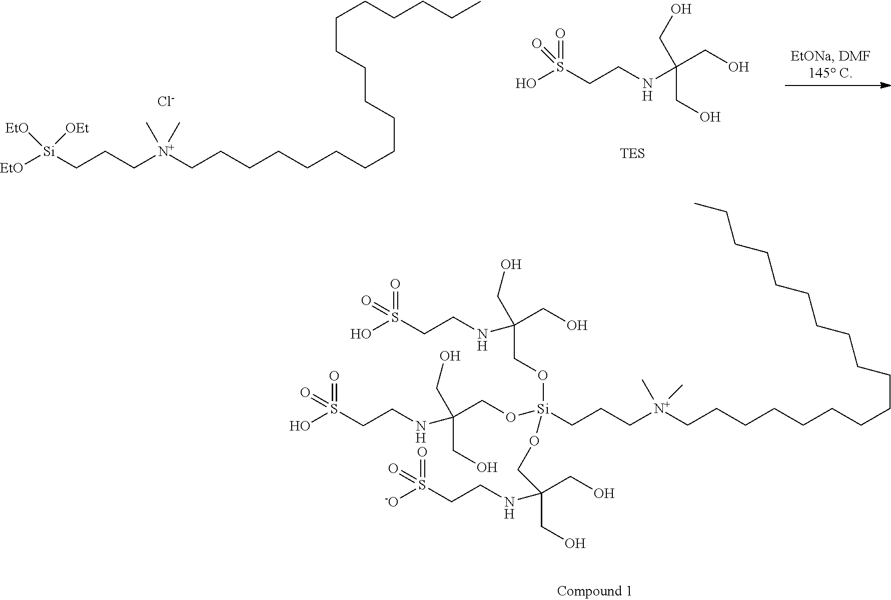

### Example 3. Synthesis of 3,3′-(((2-amino-3-hydroxy-2-(hydroxymethyl)propoxy)(3-(dimethyl(octadecyl)ammonio)propyl)silanediyl)bis(oxy))bis(1-hydroxy-2-(hydroxymethyl)propan-2-aminium) methanesulfonate

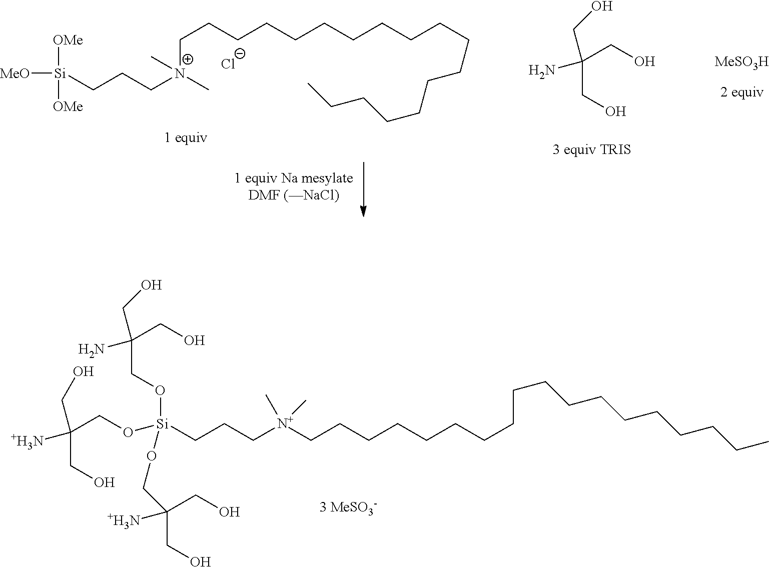 [SMILES: "MeSO3H" ; CCCCCCCCCCCCCCCCCC[N+](C)(C)CCC[Si](OC)(OC)OC ; [Cl-] ; NC(CO)(CO)CO ; CCCCCCCCCCCCCCCCCC[N+](C)(C)CCC[Si](OCC(N)(CO)CO)(OCC([NH3+])(CO)CO)OCC([NH3+])(CO)CO]

Experiment 1. A 500 mL round bottom reaction vessel is outfitted with an oil bath, stir bar, distillation head and condenser, receiving flask, oil bubbler, gas inlet that allows argon to pass through vessel and distillation setup, an immersion thermoprobe for the oil bath, and a thermoprobe for the head. The reactor is charged with 47.6 mL of a 42% solution of dimethyloctadecyl[3-(trimethoxysilyl)propyl]ammonium chloride (Aldrich), 200 mL DMF, 3 equiv. of TRIS, 2 equiv. of MeSO₃H, and 1 equiv. of Na mesylate.

The mixture is heated to 125° C. (oil temp) and the methanol is collected by distillation. At this stage the precipitate (NaCl) is removed by filtration and the DMF is concentrated to about 100 mL total volume at reduced pressure. After cooling the solution to rt, a 1 L flask under argon and a stir bar is charged with anhydrous acetonitrile and cooled to ˜10 C. The DMF solution is added slowly to rapidly stirring acetonitrile (1000 mL) to generate a white suspension of the product.

Experiment 2. A 500 ml round bottom reaction vessel is outfitted with an oil bath, stir bar, distillation head and condenser, receiving flask, oil bubbler, gas inlet that allows nitrogen to pass through vessel and distillation setup, an immersion thermoprobe for the oil bath, and a thermoprobe for the head. The reactor is charged with 300 ml DMF, 21.9 g TRIS (3 equiv.), 7.13 g (1 equiv.) sodium mesylate, 11.6 g (2 equiv.) methanesulfonic acid, and then 71.43 ml of a 42% solution of dimethyloctadecyl[3-(trimethoxysilyl)propyl]ammonium chloride (Aldrich). The mixture is heated to 80 C (oil temp) for 2 h and then heated to 120° C. and the methanol is collected by distillation. As the reaction is heated, a homogenous solution forms. As the methanol is distilled off a white precipitate begins to form. The reaction is heated until the head temperature drops and no more MeOH is evolved (about 1.5 hours). Nitrogen is then allowed to sweep through the vessel to remove MeOH and then cooled to about 80° C. Vacuum is carefully applied to distill all MeOH possible. Precipitated NaCl is then removed by centrifugation and decanting. A 1 L flask under argon and a stir bar is charged with anhydrous acetonitrile and cooled to −10° C. The DMF solution is added slowly to rapidly stirring acetonitrile to form a white suspension. Stirring is continued for 30 minutes. The colorless solid is collected on a glass frit under argon and washed with acetonitrile and then ether. The hygroscopic solid is then transferred to a vacuum flask and dried under vacuum. The yield is 50 g (81%).

### Example 4. Synthesis of N,N-dimethyl-N-(3-(trihydroxysilyl)propyl)octadecan-1-ammonium methanesulfonate via hydrolysis of dimethyloctadecyl[3-(trimethoxysilyl)propyl]ammonium chloride

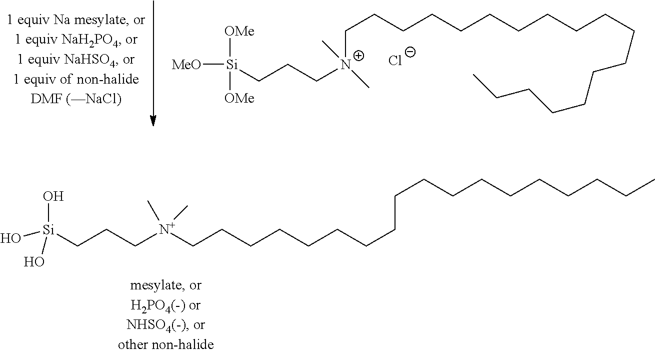 [SMILES: CCCCCCCCCCCCCCCCCC[N+](C)(C)CCC[Si](OC)(OC)OC ; [Cl-] ; CCCCCCCCCCCCCCCCCC[N+](C)(C)CCC[Si](O)(O)O]

(i) A 500 mL round bottom reaction vessel is outfitted with an oil bath, stir bar, distillation head and condenser, receiving flask, oil bubbler, gas-inlet that allows argon to pass through vessel and distillation setup, an immersion thermoprobe for the oil bath, and a thermoprobe for the head. The reactor is charged with 47.6 mL of a 42% solution of dimethyloctadecyl[3-(trimethoxysilyl)propyl]ammonium chloride (Aldrich), 200 mL DMF, 3 equiv. of H₂O and 1 equiv. of Na mesylate, or NaH₂PO₄, or NaHSO₄, or similar non-halidic counterions.
(ii) The mixture is heated to 125° C. (oil temp) and the methanol is removed from the reaction mixture via fractional distillation with a Vigereux column. After removal of the methanol the reaction is then cooled to room temperature and the resulting precipitate (NaCl) is removed by filtration.
(iii) A 1 L flask is charged with anhydrous acetonitrile and cooled to ˜10° C. The DMF solution from step (ii) is added slowly to the rapidly stirring acetonitrile to form a white suspension. Stirring is continued for 30 minutes. The precipitate is then collected by filtration.

### Example 5. Ammonium silane synthesis via methylation of tertiary amines

Synthesis of ammonium salts from their respective tertiary amines can be accomplished via methyl transfer. In certain embodiments, an ammonium silane described herein is synthesized from a tertiary amine precursor according to the following scheme:

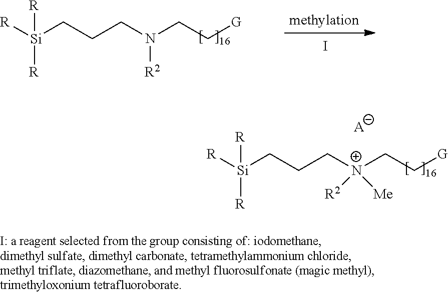

In such instances, an electrophilic methyl transfer reagent reacts with the teriary amine for direct access to the ammonium salt from the amine. These methyl transfer reagents react with tertiary amine nucleophiles to form a stabilized ion-pair. The nature of the anion can be chosen to avoid halides. This strategy will provide direct access to ammonium silane salts with benign counterions. The methyl transfer reagents (in order of increasing reactivity) for the methylation of tertiary amines in the instant invention may include alkyl halides, dimethyl sulfate, dimethyl carbonate, tetramethylammonium chloride, methyl triflate, diazomethane, methyl fluorosulfonate, trimethyloxonium tetrafluoroborate, and other electrophilic methylation reagents.

### Example 6. Ammonium silane synthesis via protonation of tertiary amines

In certain embodiments, an ammonium silane described herein is a tertiary ammonium salt, synthesized from a tertiary amine precursor according to the following scheme:

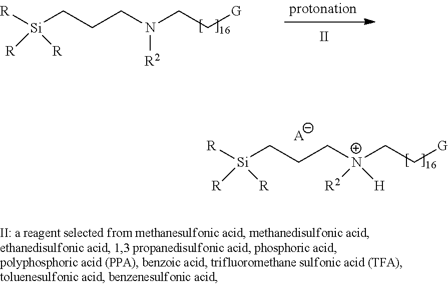

This general method is operationally simple. Ammonium silane salts described herein can contain one, two, three, or even four counter ions present for each silicon atom, the identities of which may be selected by the choice of the acid employed in this strategy. The specific charge state of the ammonium salts described herein can be predictably controlled according to molar stoichiometry of the acid and the alcohol reactants.

### Example 7. Ammonium Silane Synthesis Via Oxidation of Tertiary Amines

In certain embodiments, an ammonium silane described herein is synthesized from a tertiary amine precursor according to the following scheme:

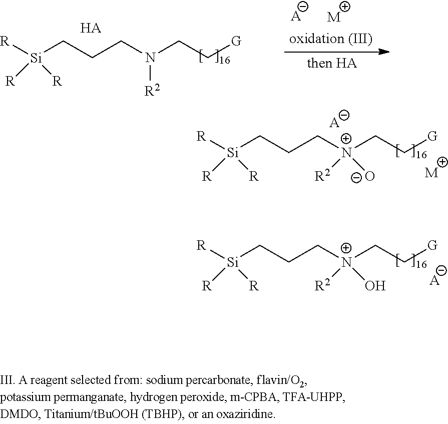

The tertiary amines can be converted to their corresponding N-oxide salts via treatment of the tertiary amine with an oxidant such as sodium percarbonate, flavin/O₂, potassium permanganate, hydrogen peroxide, m-chloroperoxybenzoic acid, TFA-UHPP, DMDO, Titanium/tBuOOH (TBHP), or an oxaziridine reagent. The resulting tertiary amine oxides may exist in various protonation states, with either an oxide anion or a hydroxyl group attached to the nitrogen. Depending on the presence of other amine functional groups in the molecule, the oxygen atom may be protonated or unprotonated. Unprotonated N-oxides may be converted to the N-hydroxy salt of an acid HA containing an A− anion as defined herein. In preferred embodiments A⁻ is not a halogen.

### Example 8. Ion-Exchange Chromatography to Access Non-Halidic Silane Salts

In other aspects a halide free compound is prepared by using ion-exchange chromatography. For example, Alcade and others have shown (Molecules 2012, 17, 4007-4027; doi:10.3390/molecules17044007) that quaternary ammonium salts can be efficiently exchanged with lactate or ibprofenate counterions. In certain embodiments the process used in this report is modified or employed in conjunction with other methods described herein to exchange different anions.

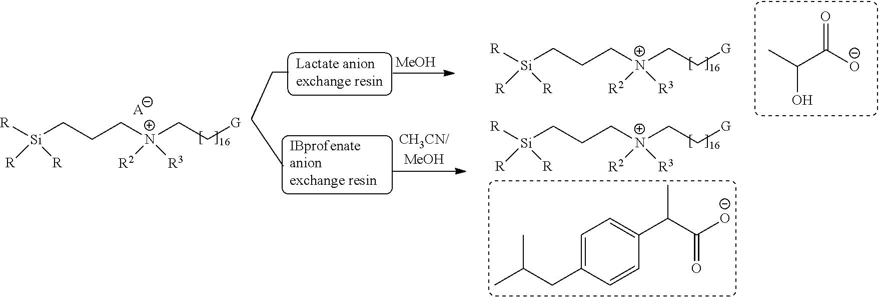

The compound mixtures produced in the experiments described herein can also be further purified of unwanted anions through this and other related processes known in the art.

### Example 9 Antimicrobial Screening Assays

The antimicrobial activity of a compound or composition described herein can be determined using any number of standard in vitro or in vivo assays known in the art. For example, the minimum inhibitory concentration (MIC) activity of the compounds described herein can be determined using the disk diffusion susceptible test (also known as the Kirby-Bauer test) or the broth dilution test, or any other suitable in vitro assay known in the art to determine the susceptibility of a microorganism to an antimicrobial. In addition, in vivo assays, for example in vivo methods for evaluating topical antimicrobial agents such as occlusion assays or rabbit eye efficacy testing may be used to characterize the antimicrobial activity of the compounds described herein.

### Disk Diffusion Susceptibility Test

One particularly suitable test is the disk diffusion susceptibility test (see Jan Hudzicki, Kirby-bauer disk diffusion susceptibility test protocol, December 2009, American Society for Microbiology, incorporated herein by reference). In the disk diffusion susceptibility test, a known concentration of a compound described herein is absorbed on a disk of filter paper (generally 6-mm) and placed on a Mueller-Hinton (MH) agar plate or other suitable agar plate used for testing the particular antimicrobial. Water is immediately absorbed into the disk from the agar and the compound begins to diffuse into the surrounding agar. The rate of diffusion through the agar is not as rapid as the rate of extraction of the compound out of the disk, therefore the concentration of the compound is highest closest to the disk and a logarithmic reduction in concentration occurs as the distance from the disk increases (see, e.g., Jorgensen, J. H., and J. D. Turnidge. 2007. Susceptibility test methods: dilution and disk diffusion methods, p. 1152-1172. In P. R. Murray, E. J. Baron, J. H. Jorgensen, M. L. Landry, and M. A. Pfaller (ed.), Manual of clinical microbiology, 9th ed. ASM Press, Washington, D.C., incorporated herein by reference). The rate of diffusion of the compound through the agar is dependent on the diffusion and solubility properties of the drug in MH agar and the molecular weight of the compound (see, e.g., Bauer, A. W., W. M. M. Kirby, J. C. Sherris, and M. Turck. 1966. Antibiotic susceptibility testing by a standardized single disk method. Am. J. Clin. Pathol. 36:493-496, incorporated herein in its entirety). Larger molecules will diffuse at a slower rate than lower molecular weight compounds. These factors, in combination, result in the compound having a unique breakpoint zone size indicating susceptibility to that compound. The disk diffusion method can also be used to test antifungals (see, for example, CLSI M44; Clinical and Laboratory Standards Institute Method for Antifungal Disk Diffusion Susceptibility Testing of Yeasts; Approved Guideline 2ndWayne: Clinical and Laboratory Standards Institute; 2009, incorporated herein). To test antifungal activity, the use of Mueller-Hinton agar supplemented with 2% glucose is recommended, providing a suitable growth for most yeasts, and 0.5 mg/L methylene blue dye medium (enhances the zone edge definition) minimizing the trailing effect. The pH of the medium should be between 7.2 and 7.4 after gelling and the agar should be 4 cm high. The inoculum is standardized to 0.5 McFarland using a densitometer and plates should be incubated at 35° C. for between 24 hours and 48 hours.

If the agar plate has been inoculated with a suspension of the pathogen to be tested prior to the placing of disks on the agar surface, simultaneous growth of the bacteria and diffusion of the compound occurs. Growth occurs in the presence of the compound when the bacteria reach a critical mass and can overpower the inhibitory effects of the compound. The estimated time of a bacterial suspension to reach critical mass is 4 to 10 hours for most commonly recovered pathogens, but is characteristic of each species, and influenced by the media and incubation temperature. The size of the zone of inhibition of growth is influenced by the depth of the agar, since the antimicrobial diffuses in three dimensions, thus a shallow layer of agar will produce a larger zone of inhibition than a deeper layer. The point at which critical mass of the antimicrobial is reached is demonstrated by a sharply marginated circle of microbial growth around the disk. The concentration of compound at this margin is called the critical concentration and is approximately equal to the minimum inhibitory concentration obtained in broth dilution susceptibility tests. Although not all fastidious or slow growing bacteria can be accurately tested by this method, the disk test has been standardized for testing streptococci, Haemophilus influenzae, and N. meningitidis through use of specialized media, incubation conditions, and specific zone size interpretive criteria (see, e.g., Clinical and Laboratory Standards Institute, Performance standards for antimicrobial disk susceptibility tests. Approved standard M2-A10, 2009, Wayne, PA Clinical and Laboratory Standards Institute, incorporated herein by reference).

### Antimicrobial Gradient Diffusion Method

Additional assays may also be used. For example, the antimicrobial gradient diffusion method uses the principle of establishment of an antimicrobial concentration gradient in an agar medium as a means of determining susceptibility (see, e.g., Reller et al., Antimicrobial Susceptibility Testing: A Review of General Principles and Contemporary Practices, Clinical Infectious Diseases, Volume 49, Issue 11, 1 Dec. 2009, Pages 1749-1755, https://doi.org/10.1086/647952). The Etest (bioMerieux AB BIODISK) is a commercial version available in the United States. It employs thin plastic test strips that are impregnated on the underside with a dried antibiotic concentration gradient and are marked on the upper surface with a concentration scale. As many as 5 or 6 strips may be placed in a radial fashion on the surface of an appropriate 150-mm agar plate that has been inoculated with a standardized organism suspension like that used for a disk diffusion test. After overnight incubation, the tests are read by viewing the strips from the top of the plate. The MIC is determined by the intersection of the lower part of the ellipse shaped growth inhibition area with the test strip.

### Broth Dilution Susceptibility Test

In addition to or in alternative, the antimicrobial activity of an ammonium silane compound or composition described herein can be determined through the use of a broth dilution susceptibility test (see, e.g., Reller et al., Antimicrobial Susceptibility Testing: A Review of General Principles and Contemporary Practices, Clinical Infectious Diseases, Volume 49, Issue 11, 1 Dec. 2009, Pages 1749-1755, https://doi.org/10.1086/647952). This procedure uses serial dilutions (usually 2X) of a compound of interest (eg, 0.25, 0.5, 1, 2, 4, 8, and 16 μg/mL) in a liquid growth medium dispensed in, e.g., test tubes or standard trays containing 96 wells. The compound-containing tubes are inoculated with a standardized suspension of the microbe, for e.g., 1-5×10⁵ CFU/mL for bacterial cultures). Following overnight incubation at, e.g., 35° C., the tubes are examined for visible microbial growth as evidenced by turbidity. The lowest concentration of antibiotic that prevents growth generally represents the minimal inhibitory concentration (MIC). The advantage of this technique is the generation of a quantitative result (i.e., the MIC).

Currently, phenotypic assays to perform in vitro anti-fungal broth dilution susceptibility tests for either yeasts or filamentous fungi (also termed molds) include two universally recognized standard methods, Clinical and Laboratory Standards Institute (CLSI) (Clinical and Laboratory Standards Institute. M27-A3: Reference Method for Broth Dilution Antifungal Susceptibility Testing of Yeasts; Approved Standard—3rd ed.; CLSI: Wayne, PA, USA, 2008.; Clinical and Laboratory Standards Institute. M38-A2: Reference Method for Broth Dilution Antifungal Susceptibility Testing of Filamentous Fungi; Approved Standard—2nd ed.; CLSI: Wayne, PA, USA, 2008, incorporated herein by reference) and the European Committee on Antimicrobial Susceptibility Testing (EUCAST) (Arendrup et al., EUCAST-AFST. EUCAST technical note on the EUCAST definitive document EDef 7.2: Method for the determination of broth dilution minimum inhibitory concentrations of antifungal agents for yeasts EDef 7.2 (EUCAST-AFST). Clin. Microbiol. Infect. 2012, 18, E246-E247; Arendrup et al., Subcommittee on Antifungal Susceptibility Testing (AFST) of the ESCMID European Committee for Antimicrobial Susceptibility Testing (EUCAST). In EUCAST Method for the Determination of Broth Dilution Minimum Inhibitory Concentrations of Antifungal Agents for Conidia Forming Moulds Version 9.3; EUCAST: Vaxjo, Sweden, 2015, incorporated herein by reference), which apply the broth microdilution method (BMD). Both measure antifungal activity, expressed as the minimum inhibitory concentration (MIC) of an antifungal drug, which indicates the minimal drug concentration that inhibits fungal growth. Despite some methodological differences (e.g., glucose concentration, inoculum size, reading endpoint, etc.) between the two, CLSI and EUCAST have been proven to yield, upon completion of testing, comparable MIC data for all classes of antifungal agents (see, e.g., Posteraro et al., The future of fungal susceptibility testing. Future Microbiol. 2014, 9, 947-967; Pfaller et al., Comparison of the broth microdilution (BMD) method of the European Committee on Antimicrobial Susceptibility Testing with the 24-hour CLSI BMD method for testing susceptibility of Candida species to fluconazole, posaconazole, and voriconazole by use of epidemiological cutoff values. J. Clin. Microbiol. 2011, 49, 845-850; Pfaller et al., Progress in antifungal susceptibility testing of Candida spp. by use of Clinical and Laboratory Standards Institute broth microdilution methods, 2010 to 2012. J. Clin. Microbiol. 2012, 50, 2846-2856, incorporated herein by reference).

### Minimum Bactericidal Concentration (MBC) Assay

Additional assays may be performed to further characterize the antimicrobial activity of an ammonium silane compound or composition described herein. For example, the minimum bactericidal concentration (MBC) or minimum lethal concentration (MLC) may be determined following the M26-A guidelines of the Clinical and Laboratory Standards Institute (Barry et el., A-26: Methods for determining Bactericidal Activity of Antimicrobial Agents; Approved Guidelines, Sept. 1999, Vol. 19(18), incorporated herein by reference. The minimum bactericidal concentration (MBC) is the lowest concentration of an antibacterial agent required to kill a particular bacterium. It can be determined from broth dilution minimum inhibitory concentration (MIC) tests by sub-culturing to agar plates that do not contain the test agent. The MBC is identified by determining the lowest concentration of antibacterial agent that reduces the viability of the initial bacterial inoculum by ≥99.9%. The MBC is complementary to the MIC; whereas the MIC test demonstrates the lowest level of antimicrobial agent that inhibits growth, the MBC demonstrates the lowest level of antimicrobial agent that results in microbial death.

### Additional Useful In Vitro Assays

Additional assays well-known in the art that may be used to test the antimicrobial activity, including antibacterial and antifungal activity, include the agar well diffusion methods, the agar plug diffusion method, the cross streak method, the poisoned food method, thin-layer chromatography bioautography, agar dilution methods, the time-kill test (time-kill curve), ATP bioluminescence test, and the flow cytofluorometric method (see Balouiri et al., Methods for in vitro evaluating antimicrobial activity: A review. Journal of Pharmaceutical Analysis, Vol. 6, No. 2, April 2016; pg. 71-79, incorporated herein by reference). Assays well-known in the art that may be used to test the anti-viral activity of the compounds described herein include cytopathic effect (CPE) inhibitory assays (see, e.g., Schmidtke et al., A rapid assay for evaluation of antiviral activity against coxsackie virus B3, influenza virus A, and herpes simplex virus type 1. J Virol Methods. 2001 June;95(1-2):133-43; Cotarelo et al., Cytopathic effect inhibition assay for determining the in-vitro susceptibility of herpes simplex virus to antiviral agents, Journal of Antimicrobial Chemotherapy, Volume 44, Issue 5, November 1999, Pages 705-708, https://doi.org/10.1093/jac/44.5.705 incorporated herein by reference).

### In Vivo Topical Assays

In addition to the in vitro assays described above, the antimicrobial efficacy activity of an ammonium silane compound or composition described herein can be characterized using suitable in vivo assays. For example, an occlusion test measures the ability of an agent to prevent the expansion of the resident microflora which occurs when an impermeable dressing is applied to the forearm (see, e.g., Leyden et al. Updated in vivo Methods for Evaluating Topical Antimicrobial Agents on Human Skin, Journal of Investigative Dermatology, 72: 165-170 (1979), incorporated herein by reference). The principle of this test is that the micro flora of the forearm skin is sparse (101 to 102 organisms per square cm). An impermeable dressing will increase surface moisture by preventing diffusional water loss and thus enhance bacterial growth. The density of resident organisms increases significantly, with counts frequently reaching millions per sq cm by 48 hr. The organisms involved in this expansion are primarily gram-positive cocci and diphtheroids. The procedure is as follows: on each arm 0.1 ml of a compound is delivered to each of two 5-cm squares (25 sq cm), by a plastic tuberculin syringe (0.1 ml). Each site is immediately covered with a 5-em square of impermeable plastic, e.g., Saran Wrap. The site is occlusively sealed by encircling the limb with plastic tape (Dermiclear). A strip of wide white-backed adhesive tape (Zonas, Johnson & Johnson) is placed between each test site to prevent the possibility of translocation of test agents and organisms from one site to another. A third site on each arm is treated with 0.1 ml of the vehicle. The control site is always prepared first to prevent its potential contamination by the test substances. After 24 hr of occlusion, the 3 sites on one arm are quantitatively sampled. The opposite arm is sampled after 48 hr.

Additional or alternative tests include the expanded flora test, persistence test, ecological shift test, and serum inactivation test, which are known in the art (see, e.g., Leyden et al. Updated in vivo Methods for Evaluating Topical Antimicrobial Agents on Human Skin, Journal of Investigative Dermatology, 72: 165-170 (1979), incorporated herein by reference).

In vivo testing to determine the efficacy of an ammonium silane compound or composition described herein for use in the eye to treat an infection are also well known. For example, the compound described herein can be tested against targeted microbial infections by induced infections in rabbit eyes, and treating the rabbit (see, generally, Deren et al., Comparison of antifungal efficacies of moxifloxacin, liposomal amphotericin B, and combination treatment in experimental Candida albicans endophthalmitis in rabbits. Can J Microbiol. 2010 January;56(1):1-7. doi: 10.1139/w09-112, incorporated herein by reference).

### Exemplary Disk Diffusion Susceptibility Test

An exemplary disk diffusion susceptibility test for testing the antibacterial activity of a compound described herein is provided below.

### Mueller-Hinton Agar

Mueller-Hinton agar (MH agar) is the standard medium for use in routine susceptibility testing of, for example, nonfastidious bacteria, including, for example, aerobic or facultative bacteria. MH agar may be purchased as prepared agar plates from Remel (Lenexa, KS), BD BBL (Franklin Lakes, NJ), or any other supplier of prepared agar plates. Follow the manufacturer's recommendation for storage of prepared plates. MH agar can also be prepared from dehydrated media available from companies such as Remel, BD BBL, or any other supplier of dehydrated media. Prepare the media according to the manufacturer's directions.

Formula for Mueller-Hinton agar per liter of purified water:

| | | |
|---|---|---|
| Beef, Infusion from | 300.0 | g |
| Casamino acid, technical | 17.5 | g |
| Starch | 1.5 | g |
| Agar | 17.0 | g |

Suspend the components listed above in 1 liter of purified water. Mix thoroughly. Heat with frequent agitation and boil for 1 minute to completely dissolve the components. Autoclave at 121° C. for 15 minutes. Dispense as desired. Allow to solidify at room temperature, then store at 4 to 8° C. Mueller-Hinton agar is stable for approximately 70 days (per Remel Technical Services, 1 Sep. 2009) from the date of preparation. If MH agar plates are prepared from dehydrated media, the plates should be poured to a depth of 4 mm (approximately 25 ml of liquid agar for 100-mm plates and 60 ml of liquid agar for 150-mm plates, but in any case to a measured depth of 4 mm. pH of the MH agar should fall between 7.2 and 7.4 at room temperature after solidification and should be tested when the media is first prepared.

### Antimicrobial Susceptibility Disks

Impregnate a disk of standard filter paper (approximately 6-mm) with a known concentration (e.g., 1 μg/ml) of a compound, for example a compound described herein in a suitable growth medium and allow to dry at 4° C.

### McFarland Standard

McFarland standards are suspensions of either barium sulfate or latex particles that allow visual comparison of bacterial density. Commercially prepared standards are available for purchase from companies such as Remel or BD BBL. These often include a Wickerham card, which is a small card containing parallel black lines. A 0.5 McFarland standard is equivalent to a bacterial suspension containing between 1×10⁸ and 2×10⁸ CFU/ml of E. coli. McFarland standard may be prepared as describe below:
1. Add a 0.5-ml aliquot of a 0.048 mol/liter BaCl₂ (1.175% wt/vol BaCl₂·2H₂O) to 99.5 ml of 0.18 mol/liter H₂SO₄ (1% vol/vol) with constant stirring to maintain a suspension.
2. Verify the correct density of the turbidity standard by measuring absorbance using a spectrophotometer with a 1-cm light path and matched cuvette. The absorbance at 625 nm should be 0.08 to 0.13 for the 0.5 McFarland standard.
3. Transfer the barium sulfate suspension in 4- to 6-ml aliquots into screw-cap tubes of the same size as those used in standardizing the bacterial inoculums.
4. Tightly seal the tubes and store in the dark at room temperature.
5. Prior to use, vigorously agitate the barium sulfate standard on a mechanical vortex mixer and inspect for a uniformly turbid appearance. Replace the standard if large particles appear. If using a standard composed of latex particles, mix by inverting gently, not on a vortex mixer.
6. As the bacterial colonies are added to the saline in the “preparation of the inoculum” step of the procedure, compare the resulting suspension to the McFarland standard. This is done by holding both the standard and the inoculum tube side by side and no more than 1 inch from the face of the Wickerham card (with adequate light present) and comparing the appearance of the lines through both suspensions. Do not hold the tubes flush against the card. If the bacterial suspension appears lighter than the 0.5 McFarland standard, more organisms should be added to the tube from the culture plate. If the suspension appears denser than the 0.5 McFarland standard, additional saline should be added to the inoculum tube in order to dilute the suspension to the appropriate density.

### Preparation of Mueller-Hinton Plate

1. Allow a MH agar plate (one for each organism to be tested) to come to room temperature. It is preferable to allow the plates to remain in the plastic sleeve while they warm to minimize condensation.
2. If the surface of the agar has visible liquid present, set the plate inverted, ajar on its lid to allow the excess liquid to drain from the agar surface and evaporate. Plates may be placed in a 35° C. incubator or in a laminar flow hood at room temperature until dry (usually 10 to 30 minutes).
3. Appropriately label each MH agar plate for each organism to be tested.

### Preparation of Inoculum

1. Using a sterile inoculating loop or needle, touch four or five isolated colonies of the organism to be tested, for example, an organism selected from Pseudomonas (Gram-negative), Proteus (genus of Gram-negative Proteobacteria), Staphylococcus (Gram-positive), MRSA (methicillin resistant S. aureus), Escherichia coli (Gram-negative), Klebsiella (Gram-negative), Enterococcus (Gram-positive), or Haemophilius influenzae,
2. Suspend the organism in 2 ml of sterile saline.
3. Vortex the saline tube to create a smooth suspension.
4. Adjust the turbidity of this suspension to a 0.5 McFarland standard by adding more organism if the suspension is too light or diluting with sterile saline if the suspension is too heavy.
5. Use this suspension within 15 minutes of preparation.

### Inoculation of the MH Plate

1. Dip a sterile swab into the inoculum tube.
2. Rotate the swab against the side of the tube (above the fluid level) using firm pressure, to remove excess fluid. The swab should not be dripping wet.
3. Inoculate the dried surface of a MH agar plate by streaking the swab three times over the entire agar surface; rotate the plate approximately 60 degrees each time to ensure an even distribution of the inoculum.
4. Rim the plate with the swab to pick up any excess liquid.
5. Discard the swab into an appropriate container.
6. Leaving the lid slightly ajar, allow the plate to sit at room temperature at least 3 to 5 minutes, but no more than 15 minutes, for the surface of the agar plate to dry before proceeding to the next step arrow indicates the path of the swab.

### Placement of the Antimicrobial Disks

Place the appropriate antimicrobial-impregnated disks on the surface of the agar. Disks should not be placed closer than 24 mm (center to center) on the MH agar plate. Ordinarily, no more than 12 disks should be placed on a 150-mm plate or more than 5 disks on a 100-mm plate. Avoid placing disks close to the edge of the plate as the zones will not be fully round and can be difficult to measure. Each disk must be pressed down with forceps to ensure complete contact with the agar surface or irregular zone shapes may occur. If the surface of the agar is disrupted in any way (a disk penetrating the surface, visible lines present due to excessive pressure of the swab against the plate during inoculation, etc.) the shape of the zone may be affected.

### Incubation of the Plates

Incubate the inoculated plates at a temperature range of 35° C.±2° C. Results are read after about 18 hours of incubation.

### Example 10. Evaluation of Polymerization Stability of Compound 1 Hydrolysis Product

The stability of Compound 1 hydrolysis product (Scheme 1) to polymerization in aqueous media was evaluated by LCMS/MS quantification of hydrolyzed trisiloxy species.

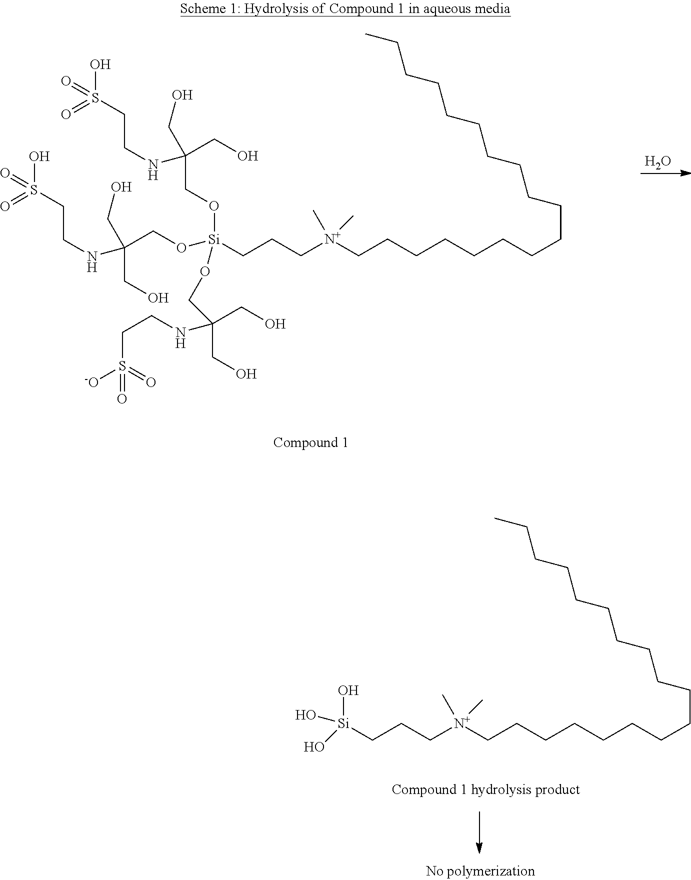

Compound 1 was diluted in water to an expected concentration of 60 μg/mL. Immediately after the solution preparation, the concentration of Compound 1 hydrolysis product in the sample was determined in triplicate by using LCMS/MS quantification, and the average of three measurements was used as a reference value. The sample was stored under ambient conditions for multiple years. Then, the concentration of Compound 1 hydrolysis product in the sample was determined again in triplicate and the average of three measurements was used to compare the measurement results with the reference value. The reported concentration corresponds to how much Compound 1 would be necessary to reach the concentration of Compound 1 hydrolysis product measured. The polymerization stability evaluation results are presented in Table 1.

TABLE 1
Results of evaluation of polymerization stability
of Compound 1 hydrolysis product

| 2019 Concentration | 2022 Concentration |
|---|---|
| of Compound 1 | of Compound 1 |
| hydrolysis | hydrolysis |
| product (μg/mL) | product (μg/mL) |
| 64.4 | 60.9 |
| 69.1 | 59.2 |
| 54.3 | 54.8 |
| Average: 62.6 | Average: 58.3 |

The results in Table 1 show that Compound 1 hydrolysis product demonstrates excellent stability to polymerization in aqueous media over prolonged periods of time.

While the present invention is described in terms of particular compounds, embodiments and uses, it is not intended that these descriptions in any way limit its scope to any such compounds, embodiments, and uses, and it will be understood that many modifications, substitutions, changes, and variations in the described compounds, embodiments, and uses, and details of the invention illustrated herein can be made by those skilled in the art without departing from the spirit of the invention, or the scope of the invention as described in the appended claims.

We claim:

1. A pharmaceutical composition comprising an excipient selected from Formula A and a compound of Formula B, wherein:
Formula A is selected from the group consisting of: 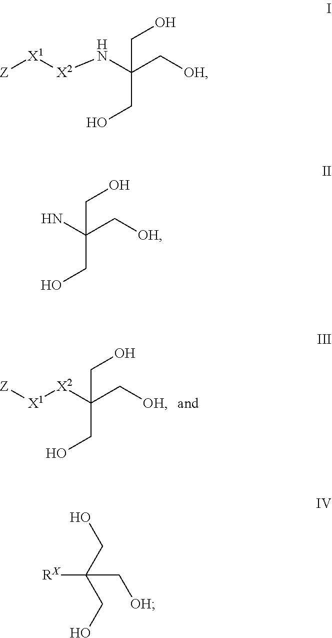
or a pharmaceutically acceptable salt thereof;
wherein in Formula A:
a) R^{X} is 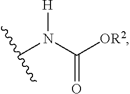
wherein R² is selected from the group consisting of hydrogen and C₁-C₆ alkyl; or
b) R^{X} is selected from 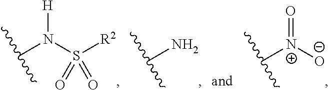
wherein R² is selected from the group consisting of hydrogen, C₁-C₆ alkyl, and —O—;
Formula B is selected from the group consisting of: 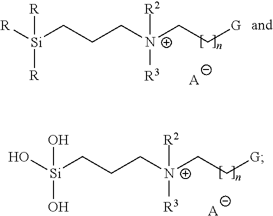
or a pharmaceutically acceptable salt thereof;
wherein in Formula B:
each R is independently selected from the group consisting of OH, alkoxy, and Cl;
G is hydrogen, alkyl, aryl, cycloalkyl, heterocycle, heteroaryl, —CN, or CF₃;
each R² is selected from the group consisting of hydrogen and C₁-C₆ alkyl;
R³ is selected from the group consisting of hydrogen, C₁-C₆ alkyl, and —O—;
X¹ is selected from the group consisting of a bond, 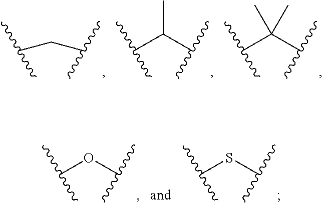
X² is selected from the group consisting of a bond, 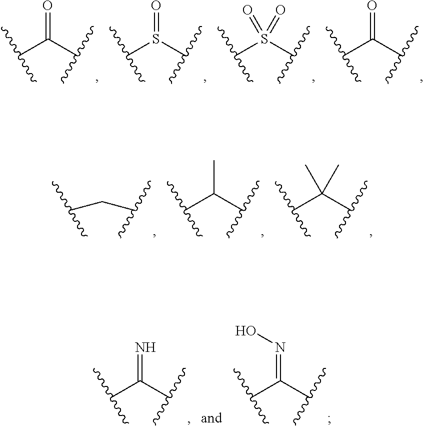
Z is selected from the group consisting of alkyl, —NO₂, —SO₃H, —SO₂H, —SO₃⁻, —SO₃M, —CO₂H, —CO₂⁻, —CO₂M, phosphate, OH, NH₂, and SH;
A⁻ is absent or a non-halogen anion;
M is a metal cation; and
each n is independently selected from the group consisting of 1, 2, 3, 4, 5, 6, 7, 8, 9, 10, 11, 12, 13, 14, 15, 16, 17, and 18.

2. The pharmaceutical composition of claim 1, wherein the excipient of Formula A is 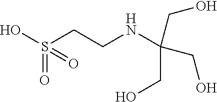 [SMILES: O=S(=O)(O)CCNC(CO)(CO)CO]
or a pharmaceutically acceptable salt thereof.

3. The pharmaceutical composition of claim 1, wherein the excipient of Formula A is 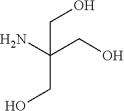
or a pharmaceutically acceptable salt thereof.

4. The pharmaceutical composition of claim 1, wherein the ratio of molecules of Formula A to molecules of Formula B is about 1.25, 1.5, 1.75, 2, 2.25, 2.5, 2.75, 3, 3.25, 3.5, 3.75, or 4 to 1.

5. The pharmaceutical composition of claim 1, wherein the ratio of molecules of Formula A to molecules of Formula B is about 2.5, 2.75, 3, 3.25, 3.5, 3.75, or 4 to 1.

6. The pharmaceutical composition of claim 1, wherein there are about 3 molecules of Formula A for every one molecule of Formula B.

7. The pharmaceutical composition of claim 1, wherein the compound of Formula B is selected from the group consisting of:

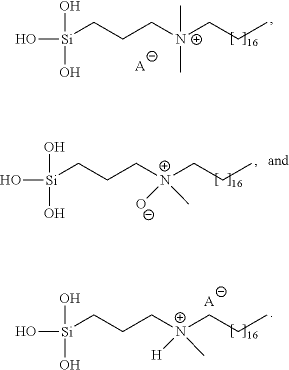

8. The pharmaceutical composition of claim 7, wherein the excipient of Formula A is 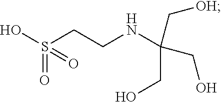 [SMILES: O=S(=O)(O)CCNC(CO)(CO)CO]
or a pharmaceutically acceptable salt thereof.

9. The pharmaceutical composition of claim 7, wherein the excipient of Formula A is 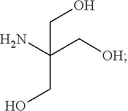
or a pharmaceutically acceptable salt thereof.

10. The pharmaceutical composition of claim 1, wherein A⁻ is selected from the group consisting of 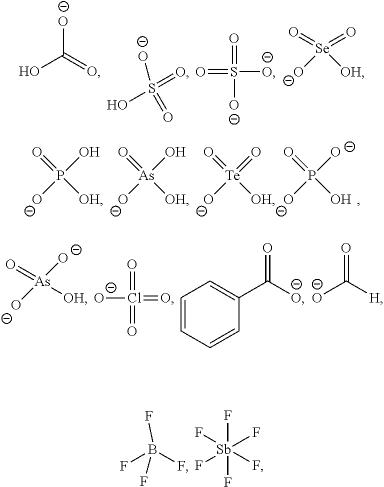
chlorite, perchlorate, hydroxide, formate, acetate, lactate, benzoate, nitrate, and salicylate anion.

11. The pharmaceutical composition of claim 1, wherein G is H.

12. The pharmaceutical composition of claim 1, wherein n is 15.

13. The pharmaceutical composition of claim 1, wherein n is 16.

14. The pharmaceutical composition of claim 1, wherein n is 17.

15. The pharmaceutical composition of claim 1, wherein n is 18.

16. The pharmaceutical composition of claim 1, wherein R² is methyl.

17. The pharmaceutical composition of claim 1, wherein R³ is methyl.

18. A method for the treatment of an infection in a human or animal host, comprising administering to the host an effective amount of pharmaceutical composition of claim 1.

19. The method of claim 18, wherein the infection is selected from the group consisting of an ocular infection, an ear infection, a skin infection, a hair infection, a nail infection, a chronic wound, a medical implant infection, a mouth infection, a nasal infection, a vaginal infection, a groin infection, a scalp infection, a uterine infection, an anal infection, and a combination thereof.

20. The method of claim 19, wherein the infection is caused by a bacterium, a fungus, an amoeba, a virus, or a combination thereof.
